(19) United States
(12) Patent Application Publication
(10) Pub. No.: US 2026/0308515 A1
(43) Pub. Date: Oct. 8, 2026

(54) GENETICALLY ENGINEERED CD4 T-CELLS FOR IN SITU SYNTHESIS OF PROTEINS

(71) Applicant: SRI International, Menlo Park, CA (US)

(72) Inventors: Parijat BHATNAGAR, Menlo Park, CA (US); Harikrishnan RADHAKRISHNAN, Menlo Park, CA (US)

(73) Assignee: SRI International, Menlo Park, CA (US)

(21) Appl. No.: 19/108,229

(22) PCT Filed: Aug. 29, 2023
(86) PCT No.: PCT/US2023/073035

§ 371 Date: Mar. 3, 2025

Related U.S. Application Data
(60) Provisional application No. 63/403,219, filed on Sep. 1, 2022.

(51) Int. Cl. C12N 5/0783; C07K 14/435; C07K 14/565; C07K 14/705; C07K 16/28; C12N 9/02; C12N 15/86

(52) U.S. Cl. CPC C12N 5/0636; C07K 14/43595; C07K 14/565; C07K 14/7051; C07K 14/70517; C07K 14/70578; C07K 16/28; C12N 9/0069; C12N 15/86; C12Y 113/12013; C07K 2319/02; C07K 2319/03; C12N 2510/02; C12N 2740/15043

(57) ABSTRACT

An example genetically engineered effector cell comprises an isolated CD4 T-cell carrying an exogenous polynucleotide sequence that includes a receptor element, an actuator element, and an effector element. The receptor element encodes a chimeric antigen receptor (CAR) comprising an extracellular antigen binding domain operably linked to a transmembrane domain, and an intracellular signaling domain, wherein the extracellular antigen binding domain recognizes a surface antigen of a target cell. The actuator element encodes a transcription factor binding site that upregulates synthesis of an effector protein. The effector element encodes the effector protein, wherein, in response to the antigen binding domain of the CAR binding to the antigen of the target cell, the genetically engineered effector cell is configured to activate and, to synthesize and secrete the effector protein.

### CROSS-REFERENCE TO RELATED APPLICATION

This PCT Application claims benefit to U.S. Provisional Application No. 63/403,219, filed Sep. 1, 2022, entitled “Primary CD4 T-Cell Biofactory for Antigen-Inducible In Situ Synthesis of Engineered Proteins”, the entirety of which is incorporated herein by reference.

### GOVERNMENT RIGHTS

This invention was made with Government support under grant numbers R21CA236640 and R33CA247739 both awarded by the National Cancer Institute of the National Institutes of Health, and under grant number DP2EB024245 awarded by the National Institute Of Biomedical Imaging and Bioengineering of the National Institutes of Health. The Government has certain rights in this invention.

### REFERENCE TO ELECTRONIC SEQUENCE LISTING

The contents of the electronic sequence listing (S1647176111_sequence listing.xml; Size: 156,336 bytes; and Date of Creation: Aug. 25, 2023) is herein incorporated by reference in its entirety.

### BACKGROUND

Various standard-of-care therapeutics are designed to treat a disease at the time of diagnosis. Although many pathogens and diseased cells undergo dynamic changes in vivo, current drugs are not designed to co-evolve along with the in vivo disease microenvironment. Such therapeutics can include drugs administered in doses that are normalized to the body weight of the patient. However, disease burden can be different for similar-sized patients, and interpatient variability can affect optimal dosing. If drug dosages are administered in excess, the therapeutic agents can end up in system circulation which can cause morbidity in normal tissue. In the case of suboptimal delivery, drug resistance may develop. While the patient can be monitored and the dosage adjusted based on health results, continuous monitoring is costly and impractical. Additionally, monitoring strategies and treatments do not exist for many diseases. Thus, static therapeutics often cannot control dynamic pathogens and diseases that evolve and/or persist. The misalignment between the dynamic disease states and static therapeutics imposes a major social and economic burden.

### SUMMARY

The present invention is directed to overcoming the above-mentioned challenges and others related to therapeutics for treating diseases, among other purposes, such as involving a genetically engineered CD4 T-cell line which can activate in situ to cause synthesis of an engineered protein (effector) against the target cell.

Various aspects of the present disclosure are directed to a genetically engineered effector cell comprising an isolated CD4 T-cell carrying an exogenous polynucleotide sequence that includes, in operative association: a receptor element that encodes a chimeric antigen receptor (CAR) including an extracellular antigen binding domain operably linked to a transmembrane domain, and an intracellular signaling domain, wherein the extracellular antigen binding domain recognizes an antigen on a surface of a target cell; an actuator element that encodes a transcription factor binding site that upregulates synthesis of an effector protein in response to the extracellular antigen binding domain of the CAR binding to the antigen of the target cell; and an effector element that encodes the effector protein, wherein, in response to the extracellular antigen binding domain of the CAR binding to the antigen of the target cell, the genetically engineered effector cell is configured to activate and, to synthesize and secrete the effector protein.

In some aspects, the effector element encodes a signal peptide operably linked to the effector protein, the signal peptide being non-native to the effector protein.

In some aspects, the genetically engineered effector cell is configured to synthesize and secrete an amount of the effector protein as a function of an amount of the target cell present in a sample or in situ.

In some aspects, the CAR is configured to cause a rise in calcium in response to the extracellular antigen binding domain binding to the antigen of the target cell and the transcription factor binding site is configured to bind to a transcription factor protein that is triggered by the rise in calcium and is translocated into the nucleus of the genetically engineered effector cell.

In some aspects, the intracellular signaling domain is selected from the group consisting of: an intracellular signaling portion of a 4-1BB, an intracellular signaling portion of a CD3 zeta, and a combination thereof.

In some aspects, the intracellular signaling domain does not include an intracellular signaling portion of CD28.

In some aspects, the transcription factor binding site is selected from the group consisting of: a nuclear factor of activated T-cell (NFAT) response element, a serum response element (SRE), a cyclic AMP response element (CRE), and a combination thereof.

In some aspects, the effector protein is selected from the group consisting of: a detectable reporter protein, a therapeutic protein, a downstream signaling protein, and a combination thereof.

In some aspects, the exogenous polynucleotide sequence includes, in operative association, the receptor element, the actuator element, and the effector element on a single construct.

In some aspects, the transmembrane domain is selected from the group consisting of: T-cell receptor α or β chain, a CD3ζ chain, CD28, CD3ε, CD45, CD4, CD5, CD8, CD9, CD16, CD22, CD28, CD33, CD37, CD64, CD80, CD86, CD134, CD137, ICOS, CD154, and a GITR.

Various aspects of the present disclosure are directed to a single construct configured to form a genetically engineered effector cell with an isolated CD4 T-cell for secretion of an effector protein upon recognition of an antigen on a surface of a target cell, the single construct comprising an exogenous polynucleotide sequence including, in operative association: a receptor element that encodes a CAR including an extracellular antigen binding domain operably linked to a transmembrane domain, and an intracellular signaling domain, wherein the extracellular antigen binding domain recognizes an antigen on a surface of a target cell; an actuator element that encodes a transcription factor binding site that upregulates synthesis of an effector protein in response to the extracellular antigen binding domain of the CAR binding to the antigen of the target cell; and an effector element that encodes the effector protein, wherein, in response to the extracellular antigen binding domain of the CAR binding to the antigen of the target cell, the genetically engineered effector cell is configured to activate and, to synthesize and secrete the effector protein.

In some aspects, the effector element encodes a signal peptide operably linked to the effector protein.

In some aspects, the single construct is carried by a viral vector or a non-viral carrier.

In some aspects, the intracellular signaling domain includes each of: an intracellular signaling portion of a 4-1BB and an intracellular signaling portion of a CD3 zeta.

In some aspects, the intracellular signaling domain does not include an intracellular signaling portion of CD28.

In some aspects, the transcription factor binding site is selected from the group consisting of: a NFAT response element, a SRE, a CRE, and a combination thereof, and the transmembrane domain is selected from the group consisting of: T-cell receptor α or β chain, a CD3 ζ chain, CD28, CD3ε, CD45, CD4, CD5, CD8, CD9, CD16, CD22, CD28, CD33, CD37, CD64, CD80, CD86, CD134, CD137, ICOS, CD154, and a GITR.

In some aspects, the effector protein is selected from the group consisting of: a detectable reporter protein, a therapeutic protein, a downstream signaling protein, and a combination thereof.

In some aspects, the exogenous polynucleotide sequence includes a sequence with at least 80% sequence identity to a sequence selected from SEQ ID NOs: 1-20.

Various aspects of the present disclosure are directed to a population of genetically engineered effector cells, each of the genetically engineered effector cells of the population comprising an isolated CD4 T-cell carrying an exogenous polynucleotide sequence that includes an actuator element bound to an effector element bound to a receptor element, wherein: a receptor element that encodes a CAR including an extracellular antigen binding domain operably linked to a transmembrane domain, and an intracellular signaling domain, wherein the extracellular antigen binding domain recognizes an antigen on a surface of a target cell; an actuator element that encodes a transcription factor binding site that upregulates synthesis of an effector protein in response to the extracellular antigen binding domain of the CAR binding to the antigen of the target cell; and an effector element that encodes the effector protein, wherein, in response to the extracellular antigen binding domain of the CAR binding to the antigen of the target cell, the population of genetically engineered effector cells is configured to activate and, to synthesize and secrete the effector protein.

In some aspects, the population of engineered effector cells are configured to activate and, in response, to synthesize and secrete a calibrated amount of the effector protein based on a presence of the target cell, the calibrated amount of the effector protein being a function of an amount of the target cell present in a plurality of cells or in a sample.

In some aspects, each effector element encodes a signal peptide operably linked to the effector protein.

In some aspects, the intracellular signaling domain is selected from the group consisting of: an intracellular signaling portion of a 4-1BB, an intracellular signaling portion of a CD3 zeta, and a combination thereof.

In some aspects, the intracellular signaling domain does not include an intracellular signaling portion of CD28.

In some aspects, the transcription factor binding site is selected from the group consisting of: a NFAT response element, a SRE, a CRE, and a combination thereof; and the transmembrane domain is selected from the group consisting of: T-cell receptor α or β chain, a CD3 ζ chain, CD28, CD3ε, CD45, CD4, CD5, CD8, CD9, CD16, CD22, CD28, CD33, CD37, CD64, CD80, CD86, CD134, CD137, ICOS, CD154, and a GITR.

In some aspects, the effector protein is selected from the group consisting of: a detectable reporter protein, a therapeutic protein, a downstream signaling protein, and a combination thereof.

Various aspects of the present disclosure are directed to a method comprising: activating a plurality of CD4 T-cells using a plurality of particles; exposing the plurality of CD4 T-cells to an exogenous polynucleotide sequence to engineer the plurality of CD4 T-cells, wherein the exogenous polynucleotide sequence includes, in operative association: a receptor element that encodes a CAR including an extracellular antigen binding domain operably linked to a transmembrane domain, and an intracellular signaling domain, wherein the extracellular antigen binding domain recognizes an antigen on a surface of a target cell; an actuator element that encodes a transcription factor binding site that upregulates synthesis of an effector protein in response to the extracellular antigen binding domain of the CAR binding to the antigen of the target cell; and an effector element that encodes the effector protein, wherein, in response to the extracellular antigen binding domain of the CAR binding to the antigen of the target cell; and expanding the activated plurality of CD4 T-cells in an expansion culture medium to form a plurality of genetically engineered effector cells comprising the plurality of CD4 T-cells carrying the exogenous polynucleotide sequence, the plurality of genetically engineered effector cells being configured to activate and, to synthesize and secrete the effector protein responsive to the extracellular antigen binding domain of the CAR binding to the antigen of the target cell.

In some aspects, the effector element further encodes a signal peptide operably linked to the effector protein.

In some aspects, the intracellular signaling domain includes: an intracellular signaling portion of a 4-1BB and an intracellular signaling portion of a CD3 zeta and/or does not include an intracellular signaling portion of CD28; the transcription factor binding site is selected from the group consisting of: a NFAT response element, a SRE, a CRE, and a combination thereof; and/or the transmembrane domain is selected from the group consisting of: T-cell receptor α or β chain, a CD3ζ chain, CD28, CD3ε, CD45, CD4, CD5, CD8, CD9, CD16, CD22, CD28, CD33, CD37, CD64, CD80, CD86, CD134, CD137, ICOS, CD154, and a GITR.

In some aspects, activating the plurality of CD4 T-cells includes exposing the plurality of CD4 T-cells to the plurality of particles loaded with anti-human CD3 and anti-human CD28 antibodies.

In some aspects, the method further includes exposing the plurality of CD4 T-cells to the plurality of particles at a cell-to-particle ratio of about 6:1 to about 1:6 for a period of time.

In some aspects, exposing the plurality of CD4 T-cells to the exogenous polynucleotide sequence includes exposing the plurality of CD4 T-cells to a vector carrying the exogenous polynucleotide sequence, wherein the vector is associated with or includes: a viral vector, a non-viral carrier, and/or lipid nanoparticles.

In some aspects, exposing the plurality of CD4 T-cells to the exogenous polynucleotide sequence includes exposing the activated plurality of CD4 T-cells to a lentivirus carrying the exogenous polynucleotide sequence.

In some aspects, exposing the plurality of CD4 T-cells to the lentivirus includes exposing between about 0.05×10⁶ cells/milliliter (mL) to about 3×10⁶ cells/mL of the plurality of CD4 T-cells to the lentivirus in a culture medium which is serum-free and contains polybrene.

In some aspects, exposing the plurality of CD4 T-cells to the exogenous polynucleotide sequence includes providing a total transformation reaction volume including a cell density of between about 0.05×10⁶ cells/mL and about 3×10⁶ cells/mL of the plurality of CD4 T-cells, a culture medium, and a vector carrying the exogenous polynucleotide sequence in defined sub-volumes for a period of time.

In some aspects, expanding the activated plurality of CD4 T-cells includes diluting a total transformation reaction volume with the expansion culture medium containing a cytokine for a period of time and at a cell density of between about 0.25×10⁶ cells/mL and about 1×10⁶ cells/mL of the activated plurality of CD4 T-cells.

In some aspects, the cytokine is selected from the group consisting of: interleukin (IL)-2, IL-7, IL-15, and a combination thereof.

Various aspects of the present disclosure are directed a method comprising: activating a plurality of T-cells using a plurality of particles; exposing the plurality of T-cells to an exogenous polynucleotide sequence in a culture medium to engineer the plurality of T-cells, wherein the exogenous polynucleotide sequence includes, in operative association: a receptor element that encodes a CAR comprising an extracellular antigen binding domain operably linked to a transmembrane domain, and an intracellular signaling domain, wherein the extracellular antigen binding domain recognizes an antigen on a surface of a target cell; an actuator element that encodes a transcription factor binding site that upregulates synthesis of an effector protein in response to the extracellular antigen binding domain of the CAR binding to the antigen of the target cell; and an effector element that encodes the effector protein, wherein, in response to the extracellular antigen binding domain of the CAR binding to the antigen of the target cell; and expanding the activated plurality of T-cells in an expansion culture medium to form a plurality of genetically engineered effector cells comprising T-cells carrying the exogenous polynucleotide sequence, the plurality of genetically engineered effector cells being configured to activate and, to synthesize and secrete the effector protein responsive to the extracellular antigen binding domain of the CAR binding to the antigen of the target cell.

In some aspects, the effector element encodes a signal peptide operably linked to the effector protein.

In some aspects, the intracellular signaling domain includes: an intracellular signaling portion of a 4-1BB and an intracellular signaling portion of a CD3 zeta and/or does not include an intracellular signaling portion of CD28; the transcription factor binding site is selected from the group consisting of: a NFAT response element, SRE, a CRE, and a combination thereof; and/or the transmembrane domain is selected from the group consisting of: T-cell receptor α or β chain, a CD3ζ chain, CD28, CD3ε, CD45, CD4, CD5, CD8, CD9, CD16, CD22, CD28, CD33, CD37, CD64, CD80, CD86, CD134, CD137, ICOS, CD154, and a GITR.

In some aspects, the plurality of T-cells include CD3 T-cells, isolated CD4 T-cells, or isolated CD8 T-cells. In some aspects, the plurality of T-cells include isolated CD4 T-cells.

In some aspects, activating the plurality of T-cells includes exposing the plurality of T-cells to the plurality of particles loaded with anti-human CD3 and anti-human CD28 antibodies.

In some aspects, the method includes exposing the plurality of T-cells to the plurality of particles at a cell-to-particle ratio of between about 6:1 and about 1:6 for a period of time.

In some aspects, the period of time includes between about 10 hours and about 36 hours.

In some aspects, the method includes resuspending the plurality of T-cells in a complete growth medium and activating the plurality of T-cells by adding the plurality of particles to the complete growth medium.

In some aspects, exposing the plurality of T-cells to the exogenous polynucleotide sequence includes exposing between about 0.05×10⁶ cells/mL to about 3×10⁶ cells/mL of the plurality of T-cells to a vector including the exogenous polynucleotide sequence in the culture medium which is serum-free and contains polybrene.

In some aspects, the culture medium contains between about 4 micrograms (μg)/mL and about 8 μg/mL of polybrene.

In some aspects, exposing the plurality of T-cells to the exogenous polynucleotide sequence includes providing a total transformation reaction volume including a cell density of between about 0.05×10⁶ cells/mL and about 3×10⁶ cells/mL of the plurality of T-cells, the culture medium, and the vector in defined sub-volumes for a period of time and at a multiplicity of infection (MOI) of between about 0.1 and about 10.

In some aspects, providing the total transformation reaction volume in the defined sub-volumes includes placing aliquots as drop volumes in a tissue-cultured well plate and placing the cultured well plate in an incubator for the period of time.

In some aspects, providing the total transformation reaction volume in the defined sub-volumes includes placing aliquots of the defined sub-volumes on a substrate having a surface which is hydrophobic or hydrophilic.

In some aspects, the total transformation reaction volume includes between about 0.5 mL and 2 mL and the sub-volumes include between about 0.05 mL and about 0.25 mL.

In some aspects, the period of time includes between about 10 hours and about 24 hours.

In some aspects, exposing the plurality of T-cells to the exogenous polynucleotide sequence includes exposing the plurality of T-cells to a vector carrying the exogenous polynucleotide sequence, wherein the vector is associated with or includes: a viral vector, a non-viral carrier, and/or lipid nanoparticles.

In some aspects, the viral vector includes a lentivirus carrying the exogenous polynucleotide sequence.

In some aspects, the lentivirus includes lentivirus particles carrying the exogenous polynucleotide sequence and the method further includes resuspending the lentivirus particles in the culture medium sufficient to achieve a MOI of between about 0.1 and about 10.

In some aspects, expanding the activated plurality of T-cells includes diluting a total transformation reaction volume with the expansion culture medium for a period of time and at a cell density of between about 0.25×10⁶ cells/mL and about 1×10⁶ cells/mL of the plurality of T-cells, wherein the expansion culture medium is a complete growth medium containing a cytokine.

In some aspects, the cytokine is selected from the group consisting of: IL-2, IL-7, IL-15, and a combination thereof. In some aspects, the cytokine includes IL-7 and IL-15.

In some aspects, the period of time includes between about 10 days and about 20 days, and the method further includes periodically changing at least a portion of the expansion culture medium over the period of time and while maintaining the cell density of between about 0.25×10⁶ cells/mL and about 1×10⁶ cells/mL.

In some aspects, the method includes adding an additive to at least one of the culture medium and the expansion culture medium, the additive being selected from the group consisting of: an antiviral inhibitor, a latency reversal agent, and a combination thereof.

Various aspects of the present disclosure are directed to population of genetically engineered effector cells comprising T-cells carrying an exogenous polynucleotide sequence formed according to the method of any of the claims.

Various aspects of the present disclosure are directed to a kit comprising: a plurality of T-cells; an exogenous polynucleotide sequence, wherein the exogenous polynucleotide sequence includes, in operative association: a receptor element that encodes a CAR comprising an extracellular antigen binding domain operably linked to a transmembrane domain, and an intracellular signaling domain, wherein the extracellular antigen binding domain recognizes an antigen on a surface of a target cell; an actuator element that encodes a transcription factor binding site that upregulates synthesis of an effector protein in response to the extracellular antigen binding domain of the CAR binding to the antigen of the target cell; and an effector element that encodes the effector protein, wherein, in response to the extracellular antigen binding domain of the CAR binding to the antigen of the target cell; a culture medium; and an expansion culture medium.

In some aspects, the effector element encodes a signal peptide operably linked to the effector protein.

In some aspects, the intracellular signaling domain includes: an intracellular signaling portion of a 4-1BB and an intracellular signaling portion of a CD3 zeta and/or does not include an intracellular signaling portion of CD28; the transcription factor binding site is selected from the group consisting of: a NFAT response element, a SRE, a CRE, and a combination thereof; and/or the transmembrane domain is selected from the group consisting of: T-cell receptor α or β chain, a CD3ζ chain, CD28, CD3ε, CD45, CD4, CD5, CD8, CD9, CD16, CD22, CD28, CD33, CD37, CD64, CD80, CD86, CD134, CD137, ICOS, CD154, and a GITR.

In some aspects, the plurality of T-cells include CD3 T-cells, isolated CD4 T-cells, or isolated CD8 T-cells.

In some aspects, the plurality of T-cells includes isolated CD4 T-cells.

In some aspects, the kit includes a plurality of particles loaded with anti-human CD3 and anti-human CD28 antibodies.

In some aspects, the kit includes another culture medium configured to resuspend the plurality of T-cells with the plurality of particles to activate the plurality of T-cells.

In some aspects, the other culture medium and the plurality of particles are configured to resuspend the plurality of T-cells at a cell-to-particle ratio of between about 6:1 and about 1:6 for a period of time of about 10 hours to about 36 hours.

In some aspects, the other culture medium is a complete growth medium.

In some aspects, the culture medium is serum-free and contains polybrene, and is configured to engineer the plurality of T-cells.

In some aspects, the culture medium contains between about 4 μg/mL and about 8 μg/mL of polybrene.

In some aspects, the kit includes a vector carrying the exogenous polynucleotide sequence, wherein the vector is associated with or includes: a viral vector, a non-viral carrier, and/or lipid nanoparticles.

In some aspects, the viral vector includes lentivirus particles carrying the exogenous polynucleotide sequence and the culture medium is configured to resuspend the lentivirus particles in the culture medium sufficient to achieve a MOI of between about 0.1 and about 10.

In some aspects, the kit includes a tissue-cultured well plate configured to receive a total transformation reaction volume including a cell density of between about 0.05×10⁶ cells/mL and about 3×10⁶ cells/mL of the plurality of T-cells, the culture medium, and the exogenous polynucleotide sequence in sub-volumes and to culture the sub-volumes for a period of time.

In some aspects, the total transformation reaction volume includes between about 0.5 mL and about 2 mL and the period of time includes between about 10 hours and about 24 hours.

In some aspects, the expansion culture medium is a complete growth medium containing a cytokine.

In some aspects, the cytokine is selected from the group consisting of: IL-2, IL-7, IL-15, and a combination thereof.

In some aspects, the cytokine includes IL-7 and IL-15.

In some aspects, the expansion culture medium is configured to dilute a total transformation reaction volume for a period of time and at a cell density of between about 0.25×10⁶ cells/mL and about 2×10⁶ cells/mL of the plurality of T-cells.

In some aspects, at least one of the culture medium and the expansion culture medium include an additive selected from the group consisting of: an antiviral inhibitor, a latency reversal agent, and a combination thereof.

### BRIEF DESCRIPTION OF THE DRAWINGS

Various example embodiments can be more completely understood in consideration of the following detailed description in connection with the accompanying drawings, in which:

FIG. 1 illustrates an example genetically engineered effector cell comprising an isolated CD4 T-cell, in accordance with the present disclosure.

FIGS. 2A-2B illustrate example genetically engineered effector cells, in accordance with the present disclosure.

FIG. 3 illustrates an example population of genetically engineered effector cells in a target environment, in accordance with the present disclosure.

FIGS. 4-5 illustrate example methods of forming genetically engineered effector cells from T-cells, in accordance with the present disclosure.

FIG. 6 illustrates an example kit for forming genetically engineered effector cells from T-cells, in accordance with the present disclosure.

FIGS. 7A-7E illustrate example polynucleotide sequences used to form genetically engineered effector cells, in accordance with the present disclosure.

FIGS. 8A-8H illustrate effects of varying different factors on production and function of the T-cell based effector cell, in accordance with the present disclosure.

FIGS. 9A-9C illustrate the results of verifying the functionality the CD4 T-cell-based effector cells as a protein delivery platform in vivo, in accordance with various embodiments.

FIGS. 10A-10H illustrate FRα-specific targeting of tumor cells by a CD4 T-cell engineered to secrete IFNβ, in accordance with the present disclosure.

FIG. 11 illustrates flow cytometry plots showing the proportion of CD4 and CD8 T-cells in the pan CD3 T-cell population from healthy donors, in accordance with the present disclosure.

FIG. 12 illustrates a comparison of CD4 and CD8 T-cell chemotaxis, in accordance with the present disclosure.

FIG. 13 illustrates an example process for forming genetically engineered effector cells from primary T-cells, in accordance with the present disclosure.

FIGS. 14A-15D illustrate example effects of various parameters on the lentivector transduction of primary T-cells, in accordance with the present disclosure.

FIGS. 16A-16E illustrate example effects of various parameters on the expansion of primary T-cells, in accordance with the present disclosure.

FIGS. 17A-17F illustrate functional validation of the effector cell formed from a primary T-cell, in accordance with the present disclosure.

FIGS. 18A-18B illustrate an example strategy for evaluating CD3 T-cell activation, in accordance with the present disclosure.

FIGS. 19A-19D illustrate example effects of additional factors on transduction of primary T-cells with lentivectors, in accordance with the present disclosure.

FIGS. 20A-20B illustrate an example exploratory screen of chemical additives for improving transduction of primary T-cells with lentivectors, in accordance with the present disclosure.

FIG. 21 illustrates an example change in the proportion of CD3 T-cell subsets in response to cytokines, in accordance with the present disclosure.

FIGS. 22A-22C illustrate example antigen-specific cytolysis and NFAT-RE inducible delivery function, in accordance with the present disclosure.

### DETAILED DESCRIPTION

In the following detailed description, reference is made to the accompanying drawings which form a part hereof, and in which is shown by way of illustration specific examples in which the disclosure can be practiced. It is to be understood that other examples can be utilized, and various changes can be made without departing from the scope of the disclosure. The following detailed description, therefore, is not to be taken in a limiting sense, and the scope of the disclosure is defined by the appended claims. It is to be understood that features of the various examples described herein can be combined, in part or whole, with each other, unless specifically noted otherwise.

Despite recent approvals by various government agencies, such as the United States Food and Drug Administration (FDA) for cell therapies, the full impact of T-cell based drugs has been limited. This is likely be due to the autologous nature of adoptive cell therapies, which can contribute to manufacturing cost, product variability, and potential for adverse events. Furthermore, the dynamic state of cell-based pathologies and inter-patient variability present challenges for optimal dosing and can require continuous monitoring of the disease state for the individual patient. As noted above, drug delivery is often administered in doses normalized to body weight and surface area. However, disease burden can be different for similar sized patients. Embodiments in accordance with the present disclosure are directed to genetically engineered effector cells comprising isolated CD4 T-cells, or other types of primary T-cells, which are used as a cellular chassis or vector to act as a biofactory for different target proteins. The engineered effector cell can be used to synthesize calibrated amounts of the target protein, and to induce autocrine and paracrine signaling due to artificial cell signaling. Such effector cells can be used, for example, as an in vivo vector for delivery target proteins in organisms, such as humans.

Embodiments of the present disclosure include primary T-cell lines that are genetically engineered with chimeric antigen receptors (CARs) to form effector cells that specifically detect (e.g., bind) antigens expressed on the surface of a target cell. In some embodiments, the effector cells are formed from or include isolated CD4 T-cells. The CD4 T-cells can be isolated from other types of cells, either prior to engineering the effector cells or during the engineering such as by using a selective expansion process. By binding to the antigen, the genetically engineered effector cells can have improved functionality from natural T-cells. Somewhat surprisingly, in experimental embodiments, CD4 T-cells had higher propensity for transduction and expansion, among higher effector secretion and activity, as compared to CD8 or CD3 T-cells when forming genetically engineered effector cells.

A T-cell can be engineered to express genetic elements including transmembrane receptor(s) that autonomously regulate the intracellular transcriptional machinery, herein sometimes referred to as an effector cell or a genetically engineered effector. Further, the genetic elements of the effector cell can be modular and/or the effector cell can include multiple genetic elements to yield an engineered effector cell having the capacity to serve as a vector for a variety of in vitro, ex vivo, and in vivo applications. Such effector cells can be modular in that parts can be conserved, and parts can be changed for different applications. For example, the modularity can be used to combine different receptor elements with different effector elements, and which allows for reprogramming the genetically engineered effector cells to target diseases with known biomarkers, such as cancer, viral infections, and/or autoimmune disorders. The genetically engineered effector cells can be used for therapeutics and treatment methods that self-regulate the therapeutic response upon stimulation by the disease cells and that are applicable to a variety of cell-based diseases, including cancers, emerging pathogens, and others that evade the immune system or involve its malfunction. Multiple types of such genetically engineered effector cells, such as genetically engineered T-cells, provide a robust, reproducible cellular system to therapeutically target complex diseases in vivo. Such genetically engineered effector cells also provide a reliable in vivo imaging technology and a reliable, in vitro sensor technology in a variety of applications.

Accordingly, in various embodiments, the genetically engineered effector cell is modular and antigen-specific. Antigen-specificity can be used to overcome tumor resistance and directs the cytolytic function toward different antigen-presenting target cells, such as host cells of a human or other organism. Further, the artificial cell-signaling pathway of such genetically engineered effectors cells can introduce the capability to serve as vector by producing calibrated amounts of protein-based therapeutics and inducting intended autocrine and paracrine signaling, upon the genetically engineered effector cell engaging the target antigen. The genetically engineered effector cell can allow for focused synthesis of the biologics at the target site and/or extend treatment duration for better patient outcome by limiting systemic toxicity. Embodiments are not limited to therapeutics, and other types of effector proteins can be produced.

Some embodiments further described herein demonstrate the successful implementation of the artificial cell-signaling pathway in a CD4 T-cell line. In some experimental embodiments, the CD4 T-cell line was transformed into a vector for engaging antigen-presenting target cells and to trigger the synthesis of calibrated amounts of engineered proteins in situ, herein sometimes referred to as “effector proteins”. The genetically engineered effector cell can provide an allogenic living vector that is modular, as described above.

As used herein, a “genetically engineered effector cell” includes and/or refers to a T-cell that is genetically engineered or modified to comprise a (i) receptor element, (ii) actuator element, and (iii) effector element, each of which can be modular. As used herein, the terms “modular” and “modularity” include and/or refer to the versatility associated with recombinant sequence domains and the resulting recombinant polypeptides when assembled in various combinations for introduction into an engineered effector cell. As used herein, “receptor element” includes and/or refers to a polynucleotide sequence encoding a transmembrane receptor, such as a CAR, capable of a specific interaction with a target cell. Depending on the particular application, the receptor element can be reprogrammed by exchanging the single chain variable fragment (scFV) portion and/or of CAR for an extracellular antigen binding domain specific for a different disease-associated antigen or other targets. Other receptor elements that can be used include, without limitation, CARs having specificity for antigens associated with autoimmune disorders, CARs having specificity for antigens associated with neural disorders (e.g., PTSD, Parkinson's disease, Alzheimer's disease), ligand-gated GPCRs (e.g., GPR1 Glucose receptor), light-gated ion channels (e.g., melanopsins, rhodopsins, photopsins), pressure sensing ion channels (e.g., TRPV1, TRPV2), and ligand-gated ion channels.

As used herein, “actuator element” includes and/or refers to a polynucleotide sequence encoding a transcription factor binding site that initiates transcription and translation events downstream of a triggering signal (e.g., binding of the sensing element to a target antigen). In general, the underlying molecular mechanism of the actuator element is based on the intracellular calcium [Ca²⁺]ᵢ dynamics, a mechanism used by almost all types of cells to regulate their functions. Exemplary response elements include, without limitation, NFAT (“nuclear factor of activated T-cells”) response element (NFAT-RE), serum response element (SRE), and cyclic AMP response element (CRE).

As used herein, “effector element” includes and/or refers to a polynucleotide sequence encoding an effector protein, and in some instances, an effector protein operably linked to a signal peptide. The polynucleotide sequence encoding the effector protein can be, for example, a sequence derived from a human gene, a sequence derived from a gene of a non-human species, a recombinant sequence, a sequence encoding a detectable reporter molecule, a sequence encoding a detectable imaging molecule, a sequence encoding a therapeutic molecule, among others.

The genetically engineered effector cell into which the receptor element, the actuator element, and the effector element are introduced can be any T-cell type including human T-cells or non-human T-cells (e.g., mammal, reptiles, plants, among others). In this manner, the genetically modified cellular “source” of the modular elements provides a cellular chassis or frame providing, among other things, transcriptional and translational machinery for expression and presentation of the receptor element, the actuator element, and the effector element. In some embodiments, the T-cells can be from a source (e.g., a first human), modified, and administered to an organism that is different than the source (e.g., the host which is a second human). In other embodiments, the T-cells can be from the source (e.g., a first human), modified, and administered back to the source (e.g., the source is the host).

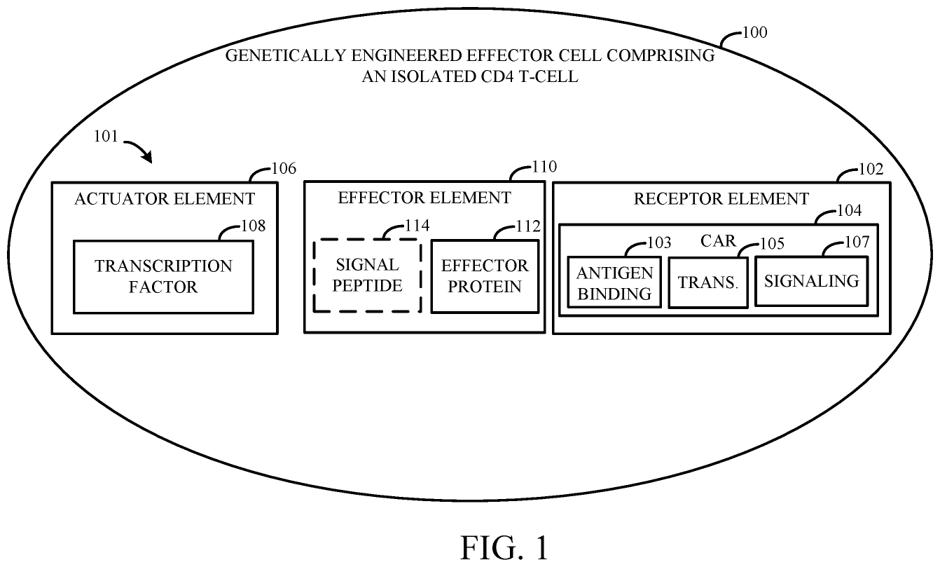

Turning now to the figures, FIG. 1 illustrates an example genetically engineered effector cell comprising an isolated CD4 T-cell, in accordance with the present disclosure. The genetically engineered effector cell 100 can be modular in that elements can be adjusted for different target cells and to synthesize different effector proteins.

As shown by FIG. 1, the genetically engineered effector cell 100 can be formed from and/or include an isolated CD4 T-cell. The CD4 T-cells can be isolated from other types of cells prior genetically modifying the T-cells or during, such as by using a selective expansion process. In some embodiments, CD3 T-cells can be used, which contain a mixture of both CD4 T-cells and CD8 T-cells generally with a higher volume of CD8 T-cells as compared to CD4 T-cell. The CD4 T-cells can then be isolated from the other cells. Somewhat surprisingly, as previously noted, CD4 T-cells have higher propensity for transduction and expansion, among higher effector secretion and activity, as compared to CD8 T-cells or CD3 T-cells when forming genetically engineered effector cells. As further described herein, CD4 T-cells grow at greater rates and/or efficiencies when isolated and then grown than when grown in a mixture, such as with CD3 T-cells.

Some embodiments include a selective expansion process involving use of particles (e.g., beads) coated with a protein (e.g., against which the engineered CD4 T-cell has a CAR or is engineered with a binding component, such as a peptide tag or short peptide sequence) and anti-CD28 antibody. This can explicitly induce the cells of interest to proliferate more. Additionally, the cells of interest (e.g., CD4 T-cells) can be separated using antibody based isolation techniques that may use flow cytometry or magnetic separation.

The genetically engineered effector cell 100 comprises an exogenous polynucleotide sequence that includes, in operative association, a receptor element 102, an actuator element 106, and an effector element 110, which can optionally include the signal peptide 114 and which optionally be on a single construct.

The receptor element 102 encodes a CAR 104. A CAR is sometimes called a “chimeric receptor”, a “T-body”, or a “chimeric immune receptor.” As used herein, a CAR includes and/or refers to an artificially constructed hybrid protein or polypeptide comprising extracellular antigen binding domain(s) 103 of an antibody (e.g., scFv) operably linked to a transmembrane domain 105 and at least one intracellular signaling domain 107. For example, the CAR 104 includes an extracellular antigen binding domain 103 operably linked to the transmembrane domain 105, and the intracellular signaling domain 107. The CAR 104 can be designed to identify a surface antigen of a target, such as a target cell of a host. The CAR 104 can mobilize internal Ca⁺² stores for intracellular Ca⁺² release in response to antigen binding. For example, the extracellular antigen binding domain 103 of the CAR 104 can recognize an antigen on a surface of a target cell, such as diseased cells of a host. The CAR 104 is configured to cause a rise in calcium in response to the extracellular antigen binding domain 103 binding to the antigen of the target cell and the transmembrane domain 105 is configured to bind to a transcription factor protein that is triggered by the rise in calcium and is translocated into the nucleus of the genetically engineered effector cell 100.

As used herein, the extracellular antigen binding domain 103 includes and/or refers to a polynucleotide sequence that is complementary to the surface antigen of the target cell. The extracellular antigen binding domain 103 can bind to the surface antigen of a target cell, as described above.

The transmembrane domain 105 includes and/or refers to a polynucleotide sequence encoding a transmembrane segment of a transmembrane protein, e.g., a type of membrane protein that spans the membrane of a cell, such as the membrane of the genetically engineered effector cell 100. The transmembrane domain 105 can be derived from a natural polypeptide, or can be artificially designed. A transmembrane domain 105 derived from a natural polypeptide can be obtained from any membrane-binding or transmembrane protein. For example, a transmembrane domain of a T-cell receptor α or β chain, a CD3ζ chain, CD28, CD3ε, CD45, CD4, CD5, CD8, CD9, CD16, CD22, CD33, CD37, CD64, CD80, CD86, CD134, CD137, ICOS, CD154, or a GITR can be used.

The intracellular signaling domain 107 includes and/or refers to a polynucleotide sequence encoding any oligopeptide or polypeptide known to function as a domain that transmits a signal to cause activation or inhibition of a biological process in a cell. Example intracellular signaling domains include an intracellular signaling portion of a CD28, an intercellular signaling portion of a 4-1BB, and an intracellular signal portion of a CD3-zeta. In some embodiments, the intracellular signaling domain 107 does not include an intracellular signaling portion of CD28. Somewhat surprisingly, the intracellular signaling portion of CD28 does not work well with the CD4 T-cell as the chassis for the genetically engineered effector cell 100. Without being bound by theory, this was surprising as CD28 is believed to exhaust cells. For example, the intracellular signaling domain 107 can include the intercellular signaling portion of 4-1BB, the intracellular signal portion of CD3-zeta, or a combination thereof. In some embodiments, the intracellular signaling domain 107 includes the intercellular signaling portion of 4-1BB and the intracellular signal portion of CD3-zeta. However, embodiments are not so limited and can include other types and combinations of intracellular signaling domains. For example, the intracellular signaling domain 107 can encode any molecule that can transmit a signal into a cell when the extracellular antigen binding domain 103 present within the same molecule binds to (interacts with) an antigen.

Generally, the extracellular antigen binding domain 103 of a CAR 104 has specificity for a particular antigen expressed on the surface of a target cell of interest. As described above, the extracellular antigen binding domain 103 capable of binding to an antigen includes any oligopeptide or polypeptide that can bind to the antigen, and includes, for example, an antigen-binding domain of an antibody and a ligand-binding domain of a receptor. The extracellular antigen binding domain 103 binds to and interacts with the antigen, for example, an antigen present on a cell surface, and thereby imparts specificity to an genetically engineered effector cell 100 expressing the CAR 104. In some embodiments, the receptor element 102 encodes a CAR 104 comprising an extracellular antigen binding domain 103 having specificity for Folate-Receptor alpha (FRα), which is an antigen found to be overexpressed on various cancers including ovarian, cervical, lung, breast, kidney, and brain. Other chimeric antigen receptors appropriate for use as the antigen binding portion of the receptor element 102 include those having specificity for a subset of immune cells, for one or more tumor antigens, and/or for one or more viral antigens.

The actuator element 106 encodes a transcription factor binding site 108. The transcription factor binding site 108 includes and/or refers to binding site for a protein that upregulates synthesis of an effector protein 112 in response to the extracellular antigen binding domain 103 of the CAR 104 binding to the antigen of the target cell. The transcription factor binding site 108 can bind to transcription factors as triggered by [Ca²⁺], which as described above, are caused to release in response to the antigen binding. In some embodiments, the transcription factor binding site 108 is selected from a nuclear factor of activated T-cell (NFAT) response element (NFAT-RE), a serum response element (SRE), a cyclic AMP response element (CRE), and a combination thereof. In some embodiments, a plurality of transcription factor binding sites can be encoded, such as between 1 and 10, between 2 and 10, between 3 and 10, between 5 and 10, between 2 and 8, between 2 and 6, between 3 and 6, 5, or 6 (e.g., 6 NFAT-REs), among other ranges or numbers. The actuator element 106 can thereby include a sequence for binding the factors triggered by [Ca²⁺], and can trigger amplified synthesis of the effector protein 112 in response to the [Ca²⁺]ᵢ rise.

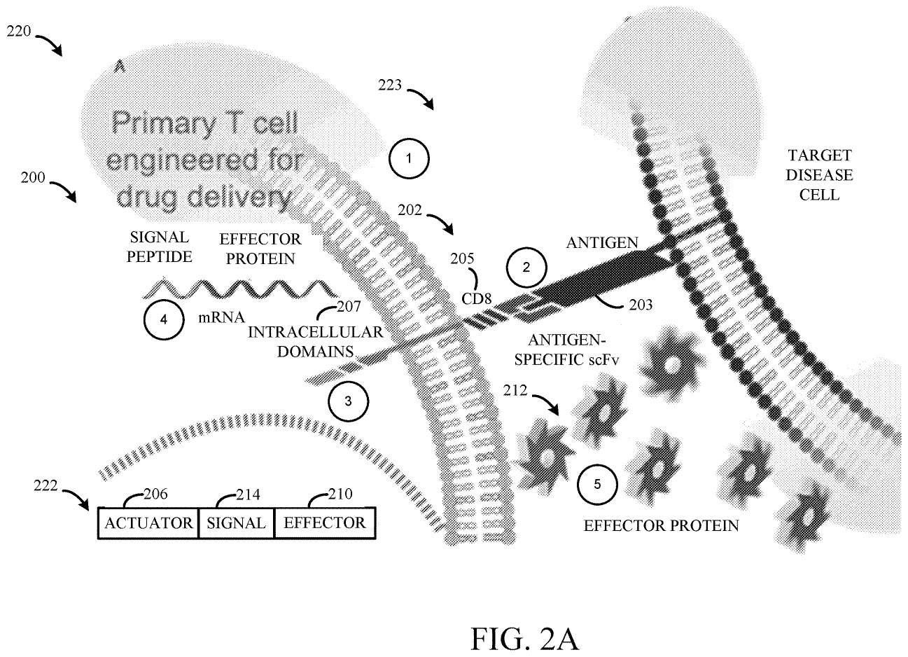

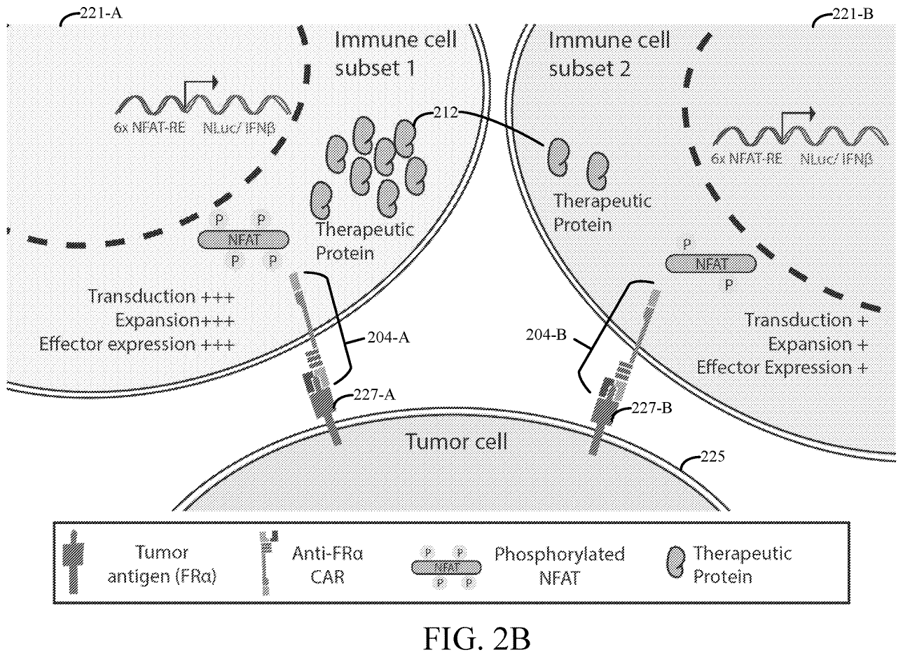

In some embodiments, the actuator element 106 encodes an NFAT transcription factor binding site for a transcription factor protein. NFAT transcription factor family consists of five members NFATc1, NFATc2, NFATc3, NFATc4, and NFAT5. For review, see Sharma S et al., PNAS, 108 (28) (2011); Hogan P G et al., Ann Rev Immunol, 28 (2010); Rao A, Hogan PG, Immunol Rev, 231 (1) (2009); Rao A, Nat Immunol, 10 (1) 2009); M. R. Müller and A. Rao, Nature Reviews Immunology, 10, 645-656 (2010); M. Oh-Hora and A. Rao, Curr. Opin. Immunol., 20, 250-258 (2008)′ and Crabtree & Olson EN, Cell 109 Suppl (2): S67-79 (April 2002), which are each hereby incorporated herein in their entireties for their teachings. NFATc1 through NFATc4 are regulated by calcium signaling. Calcium signaling is critical to NFAT activation because calmodulin, a well-known calcium sensor protein, activates the serine/threonine phosphatase calcineurin. The underlying molecular mechanism of this strategy is based on intracellular Ca²⁺ ([Ca²⁺]ᵢ) dynamics (as further shown by FIG. 2A). The [Ca²⁺]ᵢ dynamics are common to almost all cell types, and the approach is thus broadly applicable. The [Ca²⁺]ᵢ rise from CAR-mediated stimulation of cells leads to dephosphorylation of the nuclear factor of an activated effector cell 100 proteins (through Ca⁺²/calmodulin-dependent serine phosphatase calcineurin), which then translocates to the nucleus and interacts with the NFAT-RE to upregulate expression of the effector protein 112. In parallel, the NFAT-RE also performs its natural function of inducing IL-2 in the activated genetically engineered effector cell 100 that regulates clonal expansion proportional to the disease burden. The expression of a NFAT-RE induced reporter protein can also be used to quantitatively assess the level of activation of a genetically engineered effector cell 100.

The effector element 110 encodes the effector protein 112, and in some instances, encodes the effector protein 112 operably linked to a signal peptide 114. As further illustrated herein, in some embodiments, the signal peptide 114 is upstream of the effector protein 112. The signal peptide 114 can be non-native to the effector protein 112. For example, the effector protein 112 can be unable to secrete into the extracellular environment without the addition of the signal peptide 114 or can be modified to include a signal peptide 114 that allows for the effector protein 112 to secrete more efficiently than with its native signal peptide. However, embodiments are not so limited and in some embodiments, the effector protein 112 includes a native signal peptide. For example, the effector protein 112 can (natively) include the signal peptide 114.

As used herein, the terms “secretor”, “secretory peptide”, and “signal peptide” are used interchangeable and include and/or refer to a peptide that assists or directs the synthesized effector protein 112 into the extracellular environment (e.g., assists with translocating the effector element 110). The signal peptide 114 can be operably linked or fused to the effector protein 112 for release into the extracellular environment. In this manner, the signal peptide 114 can direct movement of the effector protein 112 outside of the genetically engineered effector cell 100. A signal peptide 114 is particularly advantageous when included in the genetically engineered effector cell 100 expressing an effector protein 112 that is unable to and/or minimally-able to translocate natively, where the effector protein 112 may remain inside the genetically engineered effector cell 100 in the absence of the signal peptide 114 and/or can translocate at a rate below a threshold. Generally, signal peptides are located at the N-terminus of nascent secreted proteins and characteristically have three domains: (1) a basic domain at the N-terminus, (2) a central hydrophobic core, and (3) a carboxy-terminal cleavage region. Any appropriate signal peptide can be used. For example, the signal peptide 114 can be the signal peptide of Interleukin-6 (IL-6) or Interleukin-2 (IL-2).

In various embodiments, in response to the extracellular antigen binding domain 103 of the CAR 104 binding to the antigen of the target cell (e.g., a target host cell), the genetically engineered effector cell 100 is configured to activate, and to synthesize and secrete the effector protein 112. For example, the genetically engineered effector cell 100 can synthesize and secrete an amount of the effector protein 112 as a function of an amount of the target cell present in the environment (e.g., the extracellular environment), such as secreting an amount of the effector protein 112 in the environment that is proportional to the number of target cells present in the environment.

The effector protein 112 can include a variety of different types of proteins. For example, the effector protein 112 can include a detectable reporter protein, a therapeutic protein, a downstream signaling protein, and a combination thereof. As used herein, a detectable reporter protein includes and/or refers to a protein that is detectable upon expression, such as a protein that provides an optical, electrical or other type of detectable signal. A therapeutic protein includes and/or refers to a protein that provides a therapeutic effect to the host, e.g., a patient. A downstream signaling protein includes and/or refers to a protein that drives downstream elements of a signaling pathway, such as for regulation of cell growth, proliferation, differentiation, and apoptosis.

Non-limiting examples of effector proteins include cytotoxic polypeptides of bacterial origin (e.g., parasporin, plantaricin A); insect origin (e.g., Polybia-MP1); antiviral polypeptides from viral origin (e.g., α-helical peptide (AHP)); antiviral polypeptides from viral origin (e.g., anti-viral peptides (AVP)); immunosuppressive peptides of fungal origin (e.g., colutellin A); vasodilators (e.g., relaxin, bradykinin) and endopeptidase (e.g., heparanase, relaxin, collagenase); and cell-penetrating cationic peptides (e.g., LL-37, TAT peptide). Systemic infusion of immunosuppressive agents cannot be used in hosts with these conditions due to the risk of other opportunistic infections. With respect to vasodilators and endopeptidases, such effector cells can be used to improve perfusion (see Chauhan V P & Jain R K, Nat. Mater. 12 (11): 958-962 (2013), which is incorporated herein in its entirety for its teaching) and assist in efficient delivery of anticancer agents that cannot be systemically administered as they damage structural tissues and are tumorigenic. With respect to cell-penetrating cationic peptides, these target peptides can be used to target intracellular bacteria. For example, site-specific overexpression of such peptides can be a potent therapy for tuberculosis.

As noted above, in some embodiments, the effector protein 112 is a therapeutic protein. The therapeutic protein can act directly on the target cell, in some embodiments. In other embodiments, the therapeutic protein can act on cells adjacent to the target cell or on non-cellular components. Example therapeutic proteins include a cytotoxic protein, an immunostimulatory protein, and an immunosuppressive protein.

Different parts of the genetic elements 102, 106, 110 of the genetically engineered effector cell 100 can be modular and other parts can be conserved (e.g., may not change for different implementations). For example, in some embodiments, the intracellular signaling domain 107, the actuator element 106, and the signal peptide 114 are constant domains, and the extracellular antigen binding domain 103 and the effector protein 112 are variable domains. As an example, the extracellular antigen binding domain 103 can be changed for different targets and/or the effector protein 112 can be changed to cause in situ synthesis of different proteins, while the intracellular signaling domain 107, the actuator element 106, and the signal peptide 114 remain the same for the different implementations. Keeping parts conserved can reduce production time. However, embodiments are not so limited, and any part of the genetically engineered effector cell 100 can be modified.

In some embodiments, the genetically engineered effector cell 100 can include multiple (e.g., two or more) of some or all of the genetic elements 102, 106, 110. For example, the genetically engineered effector cell 100 can include multiple receptor elements 102, multiple actuator elements 106, and/or multiple effector elements 110. In some embodiments, multiplicity takes the form of providing multiple genetically engineered effector cells (e.g., a plurality of cells) modified as described herein to a host to provide more than one task for treating or preventing a disease and/or for other purposes.

In some embodiments, the actuator element 106 is bound to the effector element 110. In some embodiments, the exogenous polynucleotide sequence 101 includes the actuator element 106 bound to the effector element 110 bound to the receptor element 102. For example, the exogenous polynucleotide sequence 101 can include the actuator element 106 bound to and upstream from the effector element 110, and the effector element 110 bound to and upstream from the receptor element 102, wherein the signal peptide 114 is upstream from the effector protein 112.

Various embodiments are directed to a (single) construct that is configured to form the genetically engineered effector cell 100 with an isolated CD4 T-cell. The single construct can comprise the exogenous polynucleotide sequence including, in operative association: (i) the receptor element 102 that encodes the CAR 104 including the extracellular antigen binding domain 103 operably linked to the transmembrane domain 105, and the intracellular signaling domain 107, wherein the extracellular antigen binding domain 103 recognizes an antigen on a surface of a target cell; (ii) the actuator element 106 that encodes the transcription factor binding site 108 that upregulates synthesis of an effector protein 112 in response to the extracellular antigen binding domain 103 of the CAR 104 binding to the antigen of the target cell; and (iii) the effector element 110 that encodes the effector protein 112, wherein, in response to the extracellular antigen binding domain 103 of the CAR 104 binding to the antigen of the target cell, the genetically engineered effector cell 100 is configured to activate and, to synthesize and secrete the effector protein 112. Further, the exogenous polynucleotide sequence can include any of the above described variations and in different combinations. In some embodiments, the single construct is carried by a viral vector (e.g., lentivector, adenovector) or a non-viral carrier or approach (e.g., a Transposon-Transposase system, Cluster Regularly Interspaced Short Palindrome Repeats (CRISPR)/Cas system, Transcription Activator-Like Nuclease (TALEN) system, Zinc Finger Nuclease (ZFN) system) that may be mediated by a transfection system (e.g., electroporation, lipid nanoparticles).

In any of the above and below described embodiments, the exogenous polynucleotide sequence can includes a sequence with at least 70% sequence identity to a sequence selected from SEQ ID NOs: 1-20, such as being selected from SEQ ID NOs: 2-9 and/or 19-20 or including SEQ ID NOs: 7 or 20. For example, the exogenous polynucleotide sequence can include at least 80%, at least 85%, at least 90%, at least 95%, at least 99%, or 100% sequence identity any of SEQ ID NOs: 1-20.

FIGS. 2A-2B illustrate example genetically engineered effector cells, in accordance with the present disclosure.

FIG. 2A illustrates an example of a genetically engineered effector cell 200 and a sequence of events 220 triggered when in a diseased environment, in accordance with the present disclosure. The genetically engineered effector cell 200 can be used as or act as a living vector to synthesize the effector protein 212 using the artificial cell-signaling pathway and/or to trigger a sequence of events 220. The genetically engineered effector cell 200 synthesizes the engineered effector protein 212 in situ upon interacting with the antigen-presenting target cell, as further described herein.

As previously described, the genetically engineered effector cell 200 comprises the receptor element 202 encoding the extracellular antigen binding domain 203, transmembrane domain 205, and the intracellular signaling domain 207, the actuator element 206 encoding the transcription factor binding site (e.g., NFAT), and the effector element 210 encoding the effector protein 212 and, optionally, the signal peptide. The genetically engineered effector cell 200 can comprise a single plasmid (e.g., a single construct 222 including each of) comprising three constant domains (e.g., the actuator element 206, the signal peptide 214, and portions of the receptor element 202, such as the transmembrane domain 205 and the intracellular signaling domain 207), and two variable domains (e.g., the extracellular antigen binding domain 203 and effector protein 212) arranged in cis.

The constant domains can be configured to provide functionality to the genetically engineered effector cell 200. The constant domains form part of the intracellular signaling pathway and include a transmembrane molecule (e.g., transmembrane domain 205) that mobilizes the calcium-dependent transcriptional machinery (e.g., actuator element 206) to upregulate the effector transgene (e.g., effector protein 212) fused to the signal peptide 214 that assists in transporting the effector transgene into the extracellular space 223.

The variable domains can be responsible for the applicability of the genetically engineered effector cell 200 to a variety of different diseases, target cells, therapy, and/or other applications. For example, the variable domains can impart specificity to the genetically engineered effector cell 200 against particular diseases. The variable domains can include molecules (e.g., a variable heavy-light (V_{H}-V_{L}) chain or scFv, variable domain of the heavy chain (VHH), a peptide, other antigens) with specificity for a biomarker on the target cell (e.g., the extracellular antigen binding domain 203 of the receptor element 202) to identify the antigen biomarker on the target cell (e.g., labeled “target disease cell”) independent of the peptide-major histocompatibility complex, and the effector transgene (e.g., effector protein 212). The variable domains are modular. For example, the extracellular antigen binding domain 203 can be exchanged or revised to reprogram the genetically engineered effector cell 200 to target biomarkers specific to different cell-based diseases. As another example, the effector protein 212 can be exchanged or revised with different therapeutic transgenes, such as for neutralizing the pathology that activated the genetically engineered effector cell 200 and essentially creating an off-shelf living vector, which is enhanced further by the innate cytolytic activity of effector cells.

In some embodiments, the receptor element 202 encodes a CAR. Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives effector cells expressing CARs the ability to recognize antigen independent of antigen processing. Referring to FIG. 2A, expression of a transmembrane CAR enables a genetically engineered effector cell 200 to sense and bind to the target antigen expressed on the surface of target cell. Binding of the CAR and target surface antigen on the target cell activates the genetically engineered effector cell 200, which triggers an activation cascade leading to the expression of the effector protein 212, such as an engineered reporter, imaging, and/or therapeutic protein. For example, expression of the effector protein 212 is autonomously expressed as part of the effector cell 200 activation cascade in response to binding of the transmembrane receptor to the antigen presented on the target cell.

More particularly, the genetically engineered effector cell 200 expressing a CAR can bind to a specific antigen via the CAR, and in response a signal is transmitted into the effector cell 200, and as a result, the effector cell 200 is activated. The activation of the effector cell 200 expressing the CAR is varied depending on the kind of target cell and an intracellular domain of the CAR, and can be confirmed based on, for example, release of a cytokine, improvement of a cell proliferation rate, change in a cell surface molecule, or the like as an index. For example, release of a cytotoxic cytokine (e.g., tumor necrosis factor, a lymphotoxin, etc.) from the activated effector cell 200 causes destruction of a target cell expressing an antigen. In addition, release of a cytokine or change in a cell surface molecule stimulates other immune cells, for example, a B-cell, a dendritic cell, a natural killer cell, and a macrophage.

As shown by FIG. 2A, an example sequence of events 220 triggered by or related to the genetically engineered effector cell 200 includes (1) the effector cell 200 actively migrating to the diseased environment, (2) the CAR on the effector cell 200 surface engaging the antigen of the target cell, (3) the effector cell 200 activates, (4) upregulation of the effector protein 212 with the signal peptide 214 through the NFAT, and (5) signal peptide 214 is cleaved off and effector protein 212 is transported into the extracellular space 223.

FIG. 2B illustrates effector cells formed from a CD4 T-cell 221-A and from a CD8 T-cell 221-B in an environment containing a target cell 225 with antigens 227-A, 227-B on the surface of the target cell 225. As shown, responsive to binding of the antigen binding domain of the CAR 204-A, 204-B of the effector cells 221-A, 221-B, the effector proteins 212 are generated. As further shown, the CD4 T-cell-based effector cell 221-A can transduce at least 3 times more, expand two times faster, and express five times more effector protein 212 than the CD8 T-cell-based effector cell 221-B. Without being bound by theory, this is believed to be because phosphorylation of ⁵³SSPS⁵⁶ motif in NFAT transactivation domain in the antigen-stimulated killer CD8 T-cell-based effector cell 221-B is impaired, which limits the ability of NFAT transcriptional machinery to signal through the NFAT-RE.

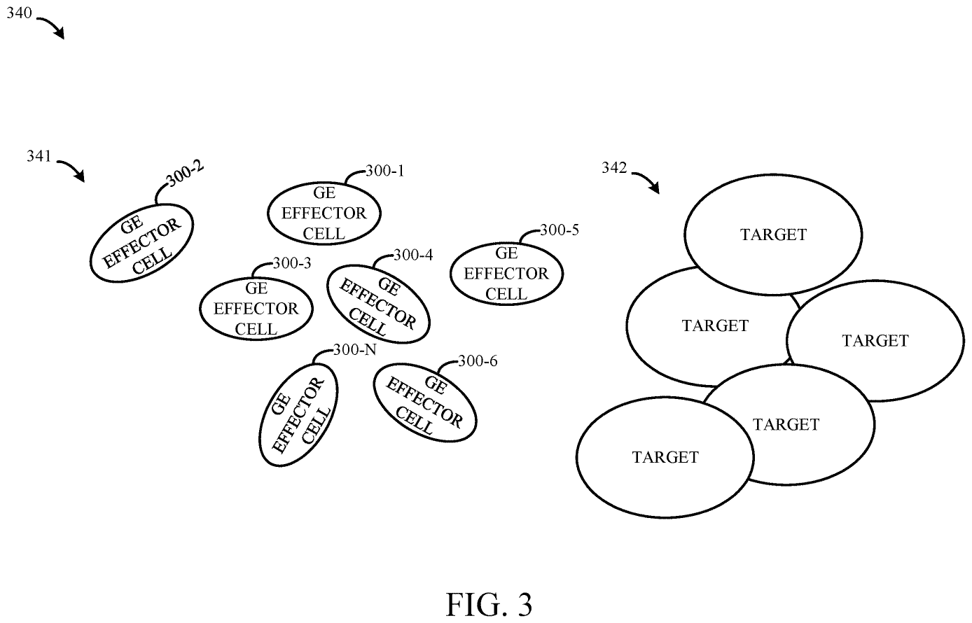

FIG. 3 illustrates an example population of genetically engineered effector cells in a diseased environment, in accordance with the present disclosure. The population 341 can include a plurality of genetically engineered effector cells 300-1, 300-2, 300-3, 300-4, 300-5, 300-6, 300-N(herein generally referend to as “the genetically engineered effector cells 300” for ease of references). Each of the genetically engineered effector cells 300 can include at least substantially the same features and elements as the genetically engineered effector cell 100 of FIG. 1, the details of which are not repeated.

In the example illustrated by FIG. 3, the environment is an extracellular space 340 that includes (a presence of) target cell(s) 342, such that the space 340 can be referred to as a diseased environment. The population 341 of the genetically engineered effector cells 300 can bind to the antigens of the target cell(s) 342 via the antigen binding domain of the CAR. In response to the binding, the genetically engineered effector cells 300 can activate and, in response, synthesize and secrete the effector protein. For example, the genetically engineered effector cells 300 can synthesize and secrete a calibrated amount of the effector protein based on a presence of the target cell(s) 342. The calibrated amount of the effector protein can be a function of an amount of the target cell 342 present in a plurality of (host) cells, such as in an extracellular space 340 or in a sample. As previously described, the calibrated amount of the effector protein can be proportional to the amount of the target cell 342. Although the extracellular space 340 illustrates genetically engineered effector cells 300 and the target cells 342, the extracellular space 340 and the plurality of (host) cells can further include other normal and/or diseased cells, among other non-cellular components. In some embodiments, each of the genetically engineered effector cells 300 can synthesize the same effector protein, and in other embodiments, different effector proteins and/or combinations thereof.

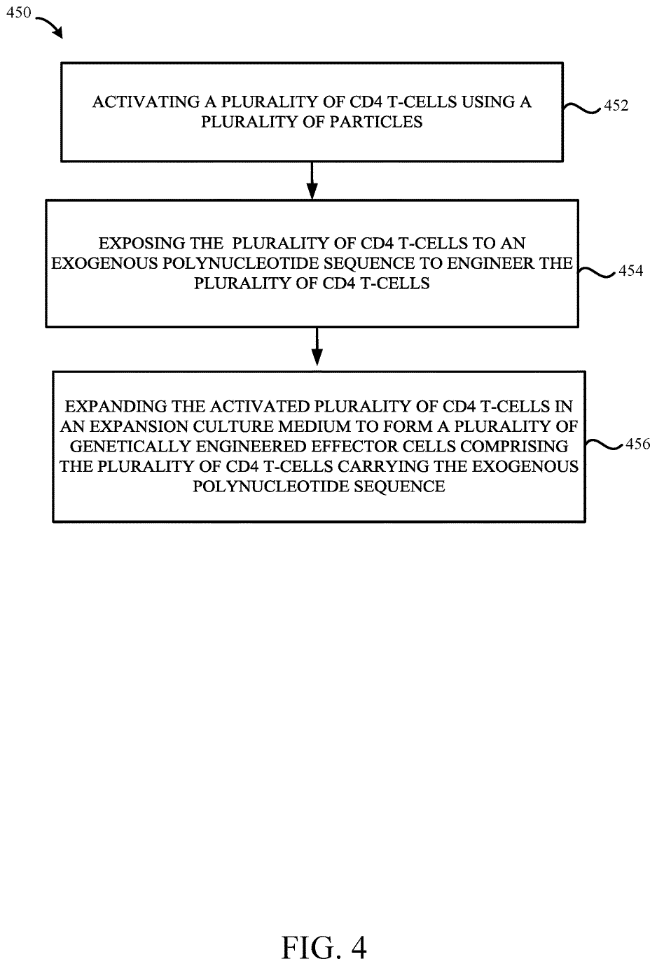

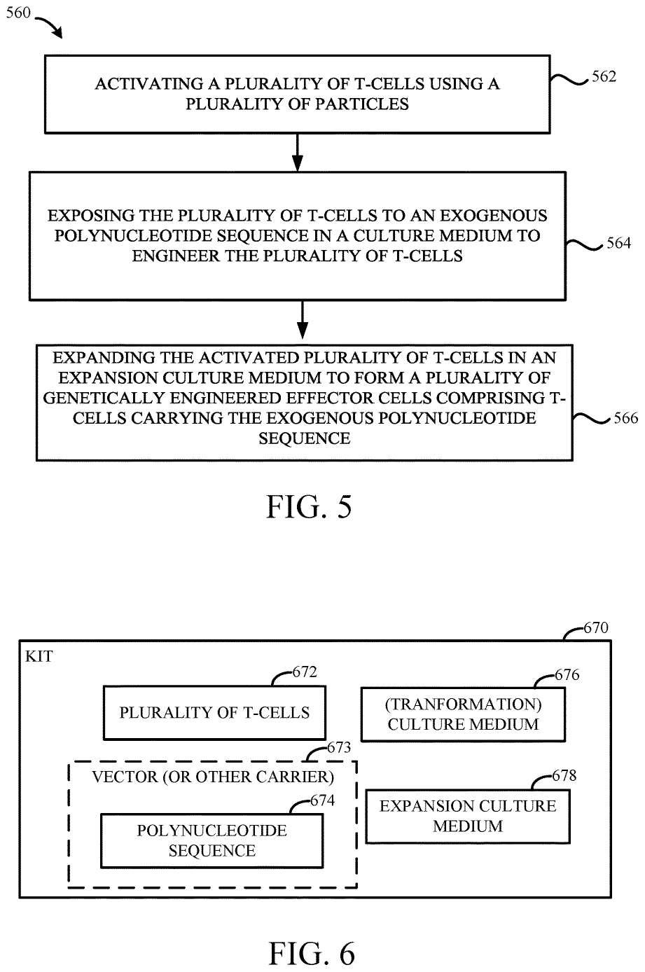

FIGS. 4-5 illustrate example methods of forming genetically engineered effector cells from T-cells, in accordance with the present disclosure. The methods 450, 560 can be implemented to form the genetically engineered effector cell 100 illustrated by FIG. 1 and/or the population 341 of genetically engineered effector cells 300 illustrated by FIG. 3. Embodiments are not limited to CD4 T-cells, such as illustrated by the method 560 of FIG. 5. The methods 450, 560 can be used to form genetically engineered effector cells from primary T-cell using select process parameters that optimized delivery performance (e.g., synthesis and secretion of the effector protein). As further described below, such process parameters can include or relate to the type of T-cell used, activation of the T-cell, transformation parameters, and expansion parameters, such but not limited transformation and expansion techniques, volumes, and/or time periods, among other parameters.

Referring to FIG. 4, at 452, the method 450 includes activating a plurality of CD4 T-cells using a plurality of particles. In some embodiments, activating the plurality of CD4 T-cells includes exposing the plurality of CD4 T-cells to the plurality of particles loaded with anti-human CD3 and anti-human CD28 antibodies. As used herein, particles include and/or refer to a localized physical object which can have or exhibit particular particle properties, such as size, shape, and/or dielectric properties. For example, the particles can include beads or nanoparticles, such as Dynabeads™. Activation of T-cells can include and/or refer to causing the T-cells to express chimeric antigen receptors on their surface, such that the T-cells are stimulated to numerically expand. The activation can occur in response to exposure to antigens. In some embodiments, the T-cells can be frozen and are thawed prior to the activation. In other embodiments, the T-cells can be fresh. In some embodiments, the T-cells can include a plurality of CD4 T-cells which are isolated from pan CD3 T-cells (e.g., CD8 T-cells are removed). In other embodiments, the T-cells can include a plurality of CD3 T-cells, with the CD4 T-cells being isolated by selective expansion to effectively remove the CD8 T-cells. In some embodiments, the plurality of CD4 T-cells can be resuspended in a complete growth medium and activated by adding the plurality of particles to the complete growth medium.

As noted above, in various embodiments, frozen T-cells can be used and in other embodiments, fresh T-cells can be used. Frozen cells can provide ease of use, as a good donor can be pre-selected. In terms of starting cell number, fresh cells can be better than frozen cells as thawing frozen cells can cause a loss of cells, such as at least a 20% loss. However, in terms of transformation efficiency, both frozen and fresh cells may be similar. Therefore, if starting cell number is not a limitation, frozen cells may be used to reduce complications in managing the logistics.

The activation can include exposing the plurality of CD4 T-cells to the plurality of particles at a particular cell-to-particle ratio and for a period of time. In some embodiment, the cell-to-particle ratio can include about 6:1 to about 1:6. In some embodiments, the cell-to-particle ratio can include about 1:2 to about 1:6, about 1:1 to about 1:6, about 1:1 to about 1:5, about 1:2 to about 1:5, about 1:3 to about 1:4, about 1:4, or about 1:3, among other ratios. In various embodiments, the cell-to-particle ratio can impact T-cell progression to peak activation, for example. The cell-to-particle ratio can define the strength of stimulation. For example, by increasing the particles coated with activation molecules, the T-cells can be stimulated with higher strength. As further described in the Experimental Embodiments, data showed the use of high cell-to-particle ratio of 1:3 activates the T-cells faster and a similar activation level can be obtained by a cell-to-particle ratio of 1:1 when stimulated for a longer period of time. Therefore, the cell-to-particle ratio can be adjusted to achieve quicker activation of T-cells. As an additional factor, excessive activation of the T-cells can cause activation induced cell death. Therefore, selecting the correct cell-to-particle ratio can be useful for optimizing activation while minimizing cell death.

In some embodiments, the period of time to activate the CD4 T-cells can include between about 10 hours to about 36 hours, between about 15 hours to about 36 hours, between about 20 hours to about 36 hours, between about 25 hours to about 36 hours, between about 30 hours to about 36 hours, between about 10 hours to about 30 hours, between about 10 hours to about 24 hours, between about 10 hours to about 20 hours, between about 15 hours to about 30 hours, between about 20 hours to about 24 hours, about 36 hours, about 30 hours, about 24 hours, about 20 hours, or about 15 hours, among other periods of time.

At 454, the method 450 includes exposing the plurality of CD4 T-cells to an exogenous polynucleotide sequence to engineer (e.g., transform and/or introduce the exogenous polynucleotide sequence into) the plurality of CD4 T-cells. The plurality of CD4 T-cells can be engineered prior to or after activating the CD4 T-cells, in various embodiments. The exogenous polynucleotide sequence can include at least some of substantially the same features and components as previously described by the genetically engineered effector cell 100 of FIG. 1, the details of which are not repeated for ease of reference.

In some embodiments, exposing the plurality of CD4 T-cells to the exogenous polynucleotide sequence can include use of a vector carrying the exogenous polynucleotide sequence. The vector can be associated with or include a viral or a non-viral carrier or approach, such as a Transposon-Transposase system, CRISPR/Cas system, TALEN system, ZFN system that may be mediated by a transfection system (e.g., electroporation, lipid nanoparticles), as previously described.

In some embodiments, exposing the plurality of CD4 T-cells to the exogenous polynucleotide sequence comprising using a virus (e.g., a viral vector), such as a lentivirus or adenovirus. In some embodiments, the CD4 T-cells are exposed to a lentivirus carrying the exogenous polynucleotide sequence. In some embodiments, the lentivirus can include lentivirus particles that carry the exogenous polynucleotide sequence. For example, the plurality of activated CD4 T-cells can be exposed to the lentivirus in a culture medium which is serum-free and contains polybrene.

Embodiments are not limited to use of a lentivirus. For example, other types of viruses or vectors can be used, such as adenoviruses. Further, the exogenous polynucleotide sequence can be introduced into the CD4 T-cells using techniques other than transduction.

The culture medium used to expose the CD4 T-cells (or other T-cells) to the exogenous polynucleotide sequence can be referred to as a “transformation culture medium”. The (transformation) culture medium, in some embodiments, can be serum-free. Serum can inhibit vector (e.g., lentivector) adhesion to cells as serum contains a multitude of proteins in excessive quantity, which can non-specifically bind to surfaces and mask protein epitopes, which are important for the virus binding and subsequent entry into target cells. Thus, it can be beneficial, in some embodiments, to not use serum to potentiate cell-to-virus interactions and improve viral transduction efficiencies. In other embodiments, the (transformation) culture medium can include a complete growth medium which contains serum (e.g., 10 percent serum) and which can be used to transduce T-cells.

In some embodiments, the serum-free culture medium can contain between about 4 micrograms (μg)/milliliter (mL) and about 8 μg/mL of polybrene; however, embodiments are not so limited and can include between about 5 μg/mL to about 12 μg/mL, about 5 μg/mL and 10 μg/mL, about 5 μg/mL and about 8 μg/mL, about 6 μg/mL and about 8 μg/mL, or about 8 μg/mL of polybrene, among other ranges. Further, in some embodiments and as noted above, the culture medium can be a complete growth medium.

In some embodiments, the transformation process can include a particular concentration of cells (e.g., cell density of 1×10⁶ cells/mL) and/or the transformation process can include confining the total transformation reaction volume (e.g., T-cells+ lentivirus or other vector carrying the exogenous polynucleotide sequence+ culture medium) in defined sub-volumes for a period of time. The total transformation reaction volume can include and/or refer to a total volume of fluid containing the T-cells, the exogenous polynucleotide sequence, and including culture medium (e.g., transformation culture medium and others fluid), such as the total volume of fluid(s) used to engineer the T-cells. The cell concentration (e.g., density of activated T-cells) and confinement of the total transformation reaction volume to sub-volumes can impact the transduction (or other type of transformation) yield by optimizing exposure to the exogenous polynucleotide sequence, such as by increasing the interaction of the virus or other vector with the CD4 T-cells. For example, exposing the plurality of CD4 T-cells to the virus (e.g., lentivirus) or other type of vector can include providing a total transformation reaction volume including a cell density of between about 0.05×10⁶ cells/mL and about 3×10⁶ cells/mL of the plurality of CD4 T-cells, the culture medium, and the lentivirus (or other vector carrying the sequence) in defined sub-volumes for a period of time. In some embodiments, the total transformation reaction volume can include the cell density of about 1×10⁶ cells/mL of the activated plurality of T-cells, the culture medium, and the lentivirus at a multiplicity of infection (MOI) of between about 0.1 and about 10. In other embodiments, the MOI can be between about 1 and about 10, about 5 and about 10, about 1 and about 8, about 5 and about 8, about 8 and about 12, or about 10, among other MOIs.

In some embodiments, the cell density of T-cells used during the transformation process, whether using a lentivirus or other viruses or vectors, can include between about 0.05×10⁶ cells/mL and about 3×10⁶ cells/mL, about 0.25×10⁶ cells/mL and about 3×10⁶ cells/mL, about 0.25×10⁶ cells/mL and about 2×10⁶ cells/mL, about 0.5×10⁶ cells/mL and about 2×10⁶ cells/mL, about 0.5×10⁶ cells/mL and about 1×10⁶ cells/mL, about 0.05×10⁶ cells/mL and about 2×10⁶ cells/mL, about 0.05×10⁶ cells/mL and about 1×10⁶ cells/mL, or about 1×10⁶ cells/mL of the plurality of CD4 T-cells (which may be activated or not), among other ranges.

The concentration of cells can impact the transduction or other transformation yield. Without being bound by theory, this may be due to the random movement of particles in a given volume of fluid increasing interactions between particles (e.g., the collision theory). It may also be central to increasing the virus-cell contact during transduction. Increasing cell concentration while keeping MOI constant in a given volume can increase virus-cell interaction due to steric reasons (with increase in virus and cell number) and can increase transduction yield.

In some embodiments, the defined sub-volumes can include drop volumes, such as spheres or other shapes which are kept in contact with the vector, such as a viral vector. In some embodiments, the total transformation reaction volume can include between about 0.5 mL and about 2 mL and the defined sub-volume can include between about 0.05 mL and about 0.25 mL. In some embodiments, the total transformation reaction volume can include about 1 mL and the defined sub-volume can include about 0.1 mL. In some embodiments, the period of time can be between about 10 hours and about 24 hours, about 10 hours and about 20 hours, about 15 hours and about 24 hours, about 15 hours and about 16 hours, or about 16 hours. Embodiments can include other variations, values, and ranges.

At 456, the method 450 includes expanding the activated plurality of CD4 T-cells in an expansion culture medium to form a plurality of genetically engineered effector cells comprising the plurality of CD4 T-cells carrying the exogenous polynucleotide sequence, the plurality of genetically engineered effector cells being configured to activate and, to synthesize and secrete the effector protein responsive to the extracellular antigen binding domain of the CAR binding to the antigen of the target cell. As used herein, cell expansion can refer to and/or include cell proliferation.

In some embodiments, the expansion culture medium can include a complete growth medium. Complete growth medium can provide the necessary nutrients at optimal proportion that enables optimal cell growth. In some embodiments, the complete growth medium can contain a cytokine. In some embodiments, the cytokine can include interleukin (IL)-2, IL-7, IL-15, or a combination thereof. For example, the expansion culture medium can include a complete growth medium containing IL-7 and IL-15.

In some embodiments, expanding the activated plurality of CD4 T-cells can includes diluting a total transformation reaction volume with the expansion culture medium containing the cytokine for a period of time and at a particular cell density of the plurality of activated and transduced plurality of CD4 T-cells. For example, the cell density can include between about 0.25×10⁶ cells/mL and about 1×10⁶ cells/mL of the plurality of activated plurality of CD4 T-cells and which is maintained over a period of time of about 14 days. Embodiments are not so limited and the period of time can be between about 10 days to about 20 days. The cell density can be between about 0.5×10⁶ cells/mL and about 1×10⁶ cells/mL, about 0.25×10⁶ cells/mL and about 0.5×10⁶ cells/mL, or about 0.5×10⁶ cells/mL, among other ranges. Further, the expansion can include periodically changing at least a portion of the expansion culture medium over the period of time and while maintaining the cell density. For example, the expansion culture medium can be changed every day, every other day, or every third day, among other times and over the period of time.

Embodiments are not limited to the above and include a variety of variations. For example, the method 450 can include adding an additive to the (transformation) culture medium and/or the expansion culture medium. The additive can include an antiviral inhibitor and/or a latency reversal agent.

In other embodiments, T-cells are not limited to CD4 T-cells and can include other types of primary T-cells such as CD3 T-cells or isolated CD8 T-cells. FIG. 5 shows such an example method 560.

At 562, the method 560 includes activating a plurality of T-cells using a plurality of particles. The plurality of T-cells can include CD3 T-cells, isolated CD4 T-cells, or isolated CD8 T-cells. The plurality of T-cells can be thawed, e.g., are frozen and thawed prior to activation, or can be fresh. In either embodiment, the method 560 can include resuspending the plurality of T-cells in a complete growth medium and activating the plurality of T-cells by adding the plurality of particles to the complete growth medium. As previously described, the plurality of T-cells can be activated by exposing the plurality of T-cells to the plurality of particles loaded with anti-human CD3 and anti-human CD28 antibodies. For example, the plurality of T-cells can be exposed to the plurality of particles at a cell-to-particle ratio of between about 6:1 and about 1:6 for a period of time, such as between about 10 hours and about 36 hours. Embodiments are not so limited and may include any of the above-described ranges and variations for the culture medium, cell-to-particle ratio, and/or periods of time, among other variations.

At 564, the method 560 includes exposing the plurality of T-cells to an exogenous polynucleotide sequence in a culture medium to engineer (e.g., transform and/or introduce the exogenous polynucleotide sequence into) the plurality of T-cells. The engineering may occur before or after the activation of the plurality of T-cells. The exogenous polynucleotide sequence can include at least some of substantially the same features and components as previously described by the genetically engineered effector cell 100 of FIG. 1, the details of which are not repeated for ease of reference. As previously described, for CD4 T-cells, the transcription binding site can be a NFAT-RE and the intracellular signaling domain may not include an intracellular portion of CD28 (e.g., may include an intracellular portion of 4-1BB and an intracellular signaling portion of a CD3 zeta). For CD8 T-cells, the transcription binding site can be SRE and/or CRE, and the intracellular signaling domain can include intracellular portion of CD28 (e.g., intracellular portion of CD28 and an intracellular signaling portion of a CD3 zeta, or intracellular portion of CD28, an intracellular portion of 4-1BB, and an intracellular signaling portion of a CD3 zeta).

As previously described, a vector can carry the exogenous polynucleotide sequence. The vector may include or be associated with a viral vector, a transposon system, or lipid nanoparticles. In some embodiments, the transformation process can include exposing the plurality of T-cells to a virus carrying the exogenous polynucleotide sequence, such as a viral vector. For example, the method 560 can include exposing the plurality of T-cells to the virus that includes a lentivirus, such as lentivirus particles carrying the polynucleotide sequence, in some embodiments. In some embodiments, exposing the plurality of T-cells to the virus includes exposing between about .05×10⁶ cells/mL and about 3×10⁶ cells/mL of the plurality of T-cells, which may be activated or not, to the virus in the culture medium which is serum-free and contains polybrene. In some embodiments, the culture medium contains between about 4 μg/mL and about 8 μg/mL of polybrene, however embodiments are not so limited.

As previously described, the transformation (e.g., transduction or other technique) can occur at particular cell concentrations and/or using defined sub-volumes. For example, exposing the plurality of T-cells to the exogenous polynucleotide sequence can include providing a total transformation reaction volume including a cell density of between about 0.05×10⁶ cells/mL and about 3×10⁶ cells/mL, among the other ranges listing about, of the plurality of T-cells, the culture medium, and the virus or other type of vector in defined sub-volumes for a period of time and at a MOI of between about 0.1 and about 10. For example, method 560 can include resuspending the lentivirus particles in the culture medium sufficient to achieve the MOI of between about 0.1 and about 10. Embodiments are not so limited and may include any of the above-described ranges for the cell concentration, vectors, total transformation volume, sub-volumes, polybrene concentration, types of culture medium, and/or MOI, among other variations described herein and combinations thereof.

In some embodiments, providing the defined sub-volumes includes placing aliquots as drop volumes in a tissue-cultured well plate and placing the cultured well plate in an incubator for the period of time. In some embodiments, in addition or alternatively, the sub-volumes can be placed on surface(s) of a substrate or substrates which are hydrophobic or hydrophilic, and the sub-volumes can include different shapes, such as spheres.

At 566, the method 560 includes expanding the activated plurality of T-cells in an expansion culture medium to form a plurality of genetically engineered effector cells comprising T-cells carrying the exogenous polynucleotide sequence, the plurality of genetically engineered effector cells being configured to activate and, to synthesize and secrete the effector protein responsive to the extracellular antigen binding domain of the CAR binding to the antigen of the target cell. In some embodiments, expanding the activated and transduced plurality of T-cells comprises diluting a total transformation reaction volume with the expansion culture medium for a period of time (e.g., 14 days) and at a cell density of about 0.25×10⁶ cells/mL and about 1×10⁶ cells/mL of the plurality of T-cells, wherein the expansion culture medium is a complete growth medium containing a cytokine. For example, the cytokine can include IL-2, IL-7, IL-15, or a combination thereof, such as IL-7 and IL-15. As previously described, the period of time can include between about 10 days and about 20 days and the method 560 further includes periodically changing at least a portion of the expansion culture medium over the period of time and while maintaining the cell density of about 0.25×10⁶ cells/mL and about 1×10⁶ cells/mL. Embodiments are not so limited and may include any of the above-described ranges for the cell concentration, expansion culture medium, and cytokines.

In some embodiments, the method 560 further include adding an additive to at least one of the (transformation) culture medium and the expansion culture medium. The additive can include an antiviral inhibitor, a latency reversal agent, and a combination thereof, as previously described.

FIG. 6 illustrates an example kit for forming genetically engineered effector cells from T-cells, in accordance with the present disclosure. The kit 670 includes a plurality of T-cells 672, an exogenous polynucleotide sequence 674, a culture medium 676, and an expansion culture medium 678. The various components 672, 673, 674, 676, 678 can include those described by any of FIGS. 1-5 herein. Not all variations are repeated here for ease of reference.

In some embodiments, for example, the plurality of T-cells 672 can include CD3 T-cells, isolated CD4 T-cells, or isolated CD8 T-cells. In some embodiments, the plurality of T-cells 672 can include isolated CD4 T-cells.

In some embodiments, the kit 670 further includes a plurality of particles loaded with anti-human CD3 and anti-human CD28 antibodies. In some embodiments, the kit 670 further includes another culture medium configured to resuspend the plurality of T-cells 672 with the plurality of particles to activate the plurality of T-cells 672. The other culture medium can include a complete growth medium. For example, the other culture medium and the plurality of particles can be configured to resuspend the plurality of T-cells at a cell-to-particle ratio of between about 6:1 and about 1:6 for a period of time of about 10 hours to about 36 hours (e.g., 24 hours).

In some embodiments, the kit 670 can further include a vector (or other carrier) 673 carrying the exogenous polynucleotide sequence 674. The vector 673 can include a viral vector, a transposon system, or lipid nanoparticles, as previously described.

In some embodiments, the vector 673 can include a viral vector, such as a lentivirus or adenovirus. For example, the vector 673 can include a lentivirus carrying the exogenous polynucleotide sequence 674, as previously described by FIG. 1. In some embodiments, the culture medium 676 can be serum-free and contains polybrene, and is configured to engineer the plurality of T-cells 672. In some embodiments, the culture medium 676 contains between about 4 μg/mL and about 8 μg/mL of polybrene. In some embodiments, the vector 673 comprises lentivirus particles carrying the exogenous polynucleotide sequence 674 and the culture medium 676 is configured to resuspend the lentivirus particles in the culture medium 676 sufficient to achieve a MOI of between about 0.1 and about 10.

In some embodiments, the kit 670 further includes a tissue-cultured well plate configured to receive a total transformation reaction volume including a cell density of between about 0.05×10⁶ cells/mL and about 3×10⁶ cells/mL of the plurality of T-cells, the culture medium, and the exogenous polynucleotide sequence in drop volumes or other sub-volumes and to culture the drop volumes or other sub-volume for a period of time. In some embodiments, other substrates are included in the kit which contains hydrophobic or hydrophilic surfaces to place the sub-volumes thereof. In some embodiments, the tissue-cultured well plate can include hydrophobic or hydrophilic surface. In some embodiments, total transformation reaction volume includes between about 0.5 mL and about 2 mL and the period of time includes between about 10 hours and about 24 hours, however embodiments are not so limited.

In some embodiments, the expansion culture medium 678 is a complete growth medium containing a cytokine. The cytokine can include IL-2, IL-7, IL-15, or a combination thereof. In some embodiments, the expansion culture medium 678 is configured to be added to and dilute a total transformation reaction volume for a period of time (e.g., 14 days) and at a cell density of between about 0.25×10⁶ cells/mL and about 1×10⁶ cells/mL of the plurality of T-cells 672.

In some embodiments, at least one of the culture medium 676 and the expansion culture medium 678 include an additive selected from the group consisting of an antiviral inhibitor, a latency reversal agent, and a combination thereof, as previously described.

The kit 670 of FIG. 6 can include any of the above described values and ranges, such as for cell concentrations, periods of time, culture mediums used, cytokines, polybrene concentrations, vectors, MOI, total transformation volume, sub-volumes, among other variations described herein and combinations thereof.

Embodiments are not limited to those illustrated by the figures and can include various variations such as method of use, different compositions, different systems and kits. For example, different variations in process parameters used the methods 450, 560 can include adjustments to the construct size, transformation methods, polybrene concentrations in media, periods of time, among other variations.

Various embodiments are directed to a pharmaceutical composition comprising a genetically engineered effector cell and a pharmaceutically acceptable carrier or excipient, such as the genetically engineered effector cell 100 of FIG. 1 and/or the population 341 of genetically engineered effector cells 300 of FIG. 3.

For example, a genetically engineered effector cell composition, such as a pharmaceutical composition, can comprises a plurality of the genetically engineered effector cells described herein and an acceptable carrier, diluents, or excipient (e.g., a pharmaceutically acceptable carrier, diluent, excipient or a combination thereof). The means of making such a composition have been described in the art (see, for instance, Remington's Pharmaceutical Sciences, 16th Ed., Mack, ed. (1980)). Preferably, the composition is prepared to facilitate the administration of the effector cells into a living organism. In some embodiments, the pharmaceutical composition comprises a plurality of genetically engineered effector cells as described herein and, for example, a balanced salt solution, preferably Hanks' balanced salt solution, or normal saline.

Some embodiments are directed to methods of forming the genetically engineered effector cells, such as genetically engineering or modifying an effector cell to include the components and features as described by the genetically engineered effector cell 100 of FIG. 1.

The genetically engineered effector cells and cell compositions provided herein have properties advantageous for use in a variety of in vitro, ex vivo, and in vivo applications, including but not limited to use as an in vivo vector for delivery of proteins for a human. For example, in vitro uses of the effector cells and cell compositions provided herein include, without limitation, detecting target cells on the basis of antigens expressed on the surface of the target cells. The target cell can be a cancer cell (e.g., tumor cell), a cell infected by a pathogen such as a virus or bacterium, a cell type associated with an autoimmune disorder (e.g., Type 1 diabetes, lupus), a cell type associated with a neurodegenerative disease such as Alzheimer's Disease, ALS, or Huntington's Disease. Also, the target (host) cell can be a cell type associated with any other pathology for which the affected (host) cell having aberrant expression of a cell surface antigen relative to an unaffected (host) cell. Methods for using the genetically engineered effector cells or cell compositions for in vitro target cell detection are described below.

Ex vivo uses of the genetically engineered effector cells and cell compositions provided herein include, without limitation, early disease detection and companion diagnostic or therapeutic applications for the disease target cells identified on the basis of antigens expressed on the surface of the disease target cells. For example, the effector cells can be used for ex vivo applications in companion diagnostics for cancer immunotherapy. By way of example, the effector cell engineered with NFAT_RE6X→Nluc-2A-GFP can be engineered to express different types of CARs. The expression of Nluc when CAR engages its target antigen versus the non-specific Nluc expression can inform on the comparative and quantitative robustness of each CAR for its efficiency to cause the intended on-target effect versus unintended off-target effects. Methods for using the genetically engineered effector cells or cell compositions in ex vivo therapeutic applications are described further below. Other ex vivo applications of the genetically engineered effector cells and cell compositions include, without limitation, applications for companion diagnostics for cell therapies for treating infectious diseases, autoimmune disorders, neurodegenerative disorders, and other cell-based pathologies associated with aberrant expression of a cell surface antigen relative to an unaffected (host) cell.

In vivo applications of the genetically engineered effector cells and cell compositions provided herein include, without limitation, in vivo imaging of disease sites, in vivo methods for localized therapy at a disease site (e.g., targeted therapy for ovarian cancer) or site of pathogen infection (e.g., targeted therapy for cells infected by dengue virus, Zika virus, West Nile virus, yellow fever, HIV, or a hepatitis virus (e.g., HepB, HepC)).

Various embodiments are directed to a panel of different types of genetically engineered effector cells, such as a plurality of effector cells engineered with different effector proteins and/or extracellular antigen binding domains (among other differences), and which are used to simultaneously target different cells and/or secrete different effector proteins.

In some embodiments, a method of detecting a target cell comprises (a) contacting a genetically engineered effector cell to a cell population, and (b) detecting expression of the effector protein, wherein detectable expression of the effector protein indicates the presence of the target cell of interest. In some embodiments, the effector cell includes a NFAT response element and a reporter protein, and in the presence of the target cell in the contacted cell population, the genetically engineered effector cell binds to a surface molecular antigen on the target cell and activates the NFAT response element; and (b) detecting expression of the reporter protein, wherein detectable expression of the reporter protein indicates the presence of the target cell.

In some embodiments, the detected target cell is a cancer cell and the antigen-binding domain of the CAR binds a cancer cell-specific surface antigen on the target cell. In other embodiments, the detected target cell is a virus-infected host cell such as, for example, a Zika virus infected cell. In some such embodiments, the surface molecular antigen expressed on the virus-infected cell can be a Zika virus-specific envelope glycoprotein (Egp). For example, the antigen-recognizing portion of the CAR is modified or exchanged to quantitatively assess different viral pathogens such as dengue virus (DENV), West Nile (WNV), and Yellow Fever (YFV). In some embodiments, the methods harness the translational machinery of the infected host cell to process viral ribonucleic acid (RNA) into a virus-specific antigen that is detectable by the genetically engineered effector cell as described herein.

Some embodiments are directed to methods of treating or preventing a disease using genetically engineered effector cells expressing a CAR as a therapeutic agent. For example, provided herein are methods comprising administering a genetically engineered effector cell expressing the CAR as an active therapeutic agent. The disease against which the effector cell expressing the CAR is administered is not particularly limited as long as the disease shows sensitivity to the effector cell. Examples of the disease include a cancer (e.g., blood cancer (leukemia), solid tumor), an inflammatory disease/autoimmune disease (e.g., asthma, eczema), hepatitis, and an infectious disease, the cause of which is a virus such as Zika virus, influenza, and HIV, a bacterium, or a fungus, for example, tuberculosis, methicillin-resistant Staphylococcus aureus (MRSA), vancomycin-resistant enterococci (VRE), and deep mycosis. In some embodiments, a genetically engineered effector cell expressing the CAR binds to an antigen expressed on the surface of a target cell that targeted to be decreased or eliminated for treatment of the aforementioned diseases, that is, a tumor antigen, a viral antigen, a bacterial antigen or the like, is administered to treat or prevent such diseases. The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, methods described herein can provide any amount of any level of treatment or prevention of cancer in a mammal. Furthermore, the treatment or prevention provided by example methods can include treatment or prevention of one or more conditions or symptoms of the disease, e.g., cancer, being treated or prevented. Also, for purposes herein, “prevention” can encompass delaying the onset of the disease, or a symptom or condition thereof.

In some embodiments, genetically engineered effector cells are administered to a host (e.g., subject) in need thereof as a composition comprising the genetically engineered effector cells and a suitable carrier, diluent, or excipient as described herein. Any appropriate method of providing modified CAR-expressing cells to a host can be used for methods described herein. In some embodiments, methods for providing effector cells to a host can be adapted from clinical protocols for cellular and adoptive immunotherapy for infusion of donor-derived immune cells into a human host. In some embodiments, an adapted clinical protocol suitable for methods provided herein comprises obtaining effector cells from a host, genetically engineering (e.g., modifying) effector cells to express a CAR and NFAT-RE regulated protein transgene as described herein, and infusing the genetically engineered effector cells back into the host. A host, as used herein, includes and/or refers to any organism, such as a human, an animal (e.g., mammal, reptile, bird), insect, plant, among others, and which can be a subject of a study or test and/or a patient.

Administration of the genetically engineered effector cells provided herein can be administered by any appropriate route, including, without limitation, administration intravenously, intratumorally, intramuscularly, subcutaneously, intraperitoneally, intra-arterially, or into an afferent lymph vessel, by parenteral administration, for example, by injection or infusion. In some embodiments, where genetically engineered effector cells or populations of such effector cells are administered, the effector cells can be cells that are allogeneic or autologous to the host, such as a mammal. Preferably, the effector cells are autologous to the host.

In some embodiments, a host to which genetically engineered effector cells are provided is monitored or assessed for increased (e.g., improved, more robust) tumor clearance. Accordingly, various embodiments are directed to methods used for cancer therapies. In some embodiments, a host to which genetically engineered effector cells are provided is monitored or assessed for clearance of cells expressing a particular antigen.

Some embodiments are directed to a method for cell-based treatment or prevention against a pathogen of interest. For such methods, the genetically engineered effector cell comprises a polynucleotide sequence encoding a therapeutic protein place of, or in addition to, the polynucleotide sequence encoding the detectable reporter protein; and is fused with a signal peptide (sec) on the 3′ end of the polynucleotide sequence to assist in extracellular transport. Upon triggering the cascade effector cell activation events and activation of the NFAT response element, expression of a therapeutic protein is induced. The method can include the localized production of a therapeutic protein at the site of the target cell (e.g., a tumor cell, infected cell) and extracellular secretion of the therapeutic protein in the disease microenvironment.

Some embodiments are directed to methods for using genetically engineered effector cells as a sensor technology in a variety of applications. By way of example, transfusion-mediated spread of emerging flavivirus pathogens, e.g., Zika virus (ZIKV), dengue virus (DENV), has been identified as a serious risk. In order to protect donated blood supply, screening of donors that includes blood testing has been recommended. Clinical symptoms manifest in only 20% of ZIKV infections, and there are no reliable commercially available ZIKV diagnostic test kits for use outside the clinical laboratory. Identifying the infection is therefore challenging, especially given the similarity of symptoms with those of other diseases and the cross-reactivity of antibodies with other arboviruses (e.g., dengue, chikungunya). Accordingly, provided herein is a method comprising contacting a genetically engineered effector cell comprising a CAR having an antigen binding domain for detection and binding to an antigen specific to the virus of interest to a sample comprising or suspected of comprising cells infected with the virus of interest, and NFAT-RE regulated reporter transgene to inform the presence of the cells infected with the virus of interest.

Additional applications of the genetically engineered effector cells described herein include the following:

To target anticancer chemotherapeutic prodrugs to a tumor location, genetically engineered effector cells can be loaded with enzymatically activatable prodrugs, where the drug-activating enzyme is synthesized only at the tumor location, thus providing localized transformation of the prodrug into its active form. In some embodiments, the prodrug may not be loaded into the effector cells, and can be infused in multiple doses subsequent to the infusion of the genetically engineered effector cells. The prodrug can alternatively be bound to an imaging nanoparticle or other means of image-guided means of active drug delivery. Attaching the prodrug to an imaging nanoparticle or engineering the effector cells to express imaging transgenes enables the engineered effector cells to guide appropriate staging of the patient in preparation of surgery and for visually identifying and/or imaging tumor margins to assist in cytoreductive surgery.

Some embodiments are directed to methods of localized delivery of a chemotherapeutic agent to a site of the disease (e.g., tumor mass, site of autoimmune disease) comprises contacting a genetically engineered effector cell to a host cell population, wherein the genetically engineered effector cell comprises (i) an exogenous polynucleotide sequence encoding a CAR comprising an antigen binding domain, a transmembrane domain, and an intracellular signaling domain; and (ii) a NFAT response element operably linked to a polynucleotide sequence encoding an enzyme, wherein, in the presence of the target host cell in the contacted cell population, the genetically engineered effector cell binds to a surface molecular antigen on the target host cell and activates the NFAT response element to initiate expression of the enzyme, which acts on the prodrug predesigned to be activated by this enzyme and uses it membrane permeability due to its hydrophobicity to be released at the site of the disease.

With regard to surgical interventions for treating cancer, genetically engineered effector cells can be used for visualizing and/or imaging tumor margins via the expression of detectable reporter protein such as fluorescent proteins (e.g., GFP, RFP, YFP, and variants thereof) or bioluminescent enzymes (e.g., luciferase). For example, such genetically engineered effector cells can be used to mark tumor margins to aid in surgical excision and to identify any residual positive tumor margins.

In some embodiments, genetically engineered effector cells are used for non-invasive detection and imaging of tumors based on expression of an imaging enzyme (e.g., thymidine kinase is capable of trapping a radioactive probe or otherwise detectable probe; tyrosinase detected by photoacoustic imaging or magnetic resonance imaging) expressed when tumor-specific CAR effector cells engage the antigen on tumor cells.

In some embodiments, genetically engineered effector cells can be used to circumvent safety concerns associated with vaccines against flaviviruses. By way of example, antigenic diversity among the four different dengue virus serotypes is responsible for the lack of antibody-mediated immunity and allows for multiple sequential infections. Although antibodies are effective in primary infection, their sub-neutralizing level during the secondary infections has been found to exacerbate the hemorrhagic fever by activating the complement system against the large infected cell mass in acute-phase. Prior dengue infection has also been found to worsen Zika infection. The use of effector cells can circumvent these safety concerns with flaviviruses because the effector cells, as described herein, can be engineered to express an antiviral protein, from human or non-human or synthetic origin, upon detecting the viral E glycoprotein (Egp) expressed on the surface of cells infected by the virus.

In some embodiments, genetically engineered effector cells comprise a CAR that detects a cancer-specific antigen on a target cancer cell (e.g., a HPV E6 or E7 antigen in case of cervical cancer) and a NFAT-RE to drive the expression of a reporter protein as described above. Such embodiments can be used for early detection of cancer.

In some embodiments, the genetically engineered effector cells comprise a CAR that detects an antigen on a pathogen-infected cell (e.g., detecting a ZIKV or DENV E glycoprotein on Zika- or dengue virus-infected cell) and a NFAT response element to induce expression of a reporter polypeptide. Such embodiments can be used for transfusion medicine to detect the presence of emerging pathogens (e.g., Zika, dengue, West Nile, Yellow Fever).

Different CARs can be used in genetically engineered effector cells with NFAT-RE regulated reporters to detect and measure signal-to-noise ratio to guide the selection of appropriate CARs for a cell-based therapy that exert the intended therapeutic effect without exhibiting unintended side-effects.

Mammalian cells can be engineered as effector cells to comprise a glucose-sensing GPCR (GPR1) which mobilizes internal Ca²⁺ stores and NFAT response element-regulated to express engineered insulin. Such engineered effector cells can be used for autonomous synthesis of insulin upon sensing glucose. Such embodiments can be used for beta-cell replacement therapy.

Other non-limiting example uses of the genetically engineered effector cells include: i) imaging of the location of disease microenvironments to assist in surgical resection or monitor disease progression/regression; ii) cytotoxicity to kill the disease cells; iii) proliferation to enhance T-cell persistence; iv) immune-stimulation to recruit other immune cells; v) chemokine to recruit other immune cells; vi) immunosuppression to create localized immunosuppressive microenvironment; and vii) regeneration to enhance tissue healing.

The various ranges provided herein include the stated range and any value or sub-range within the stated range. Furthermore, when “about” is utilized to describe a value and/or range, this includes, refers to, and/or encompasses variations (up to +/−10%) from the stated value and/or range.

As used herein, a target cell (sometimes herein interchangeably referred to as a “target cell of a host”, “target cell of interest”, “a diseased cell”, or “a target disease cell”) includes and/or refers to a cell of interest associated with a living organism (e.g., a biological component of interest). An antigen of the target cell includes and/or refers to a structure (e.g., binding site) of the target cell which the antigen binding domain of the receptor element can bind to (e.g., has an affinity for). The effector cell can be from a variety of different type of cells, such as human and non-human cells, and sometimes herein referred to as “the source”. As used herein, the terms “genetically modified” and “genetically engineered” are used interchangeably and include and/or refer to a prokaryotic or eukaryotic cell that includes an exogenous polynucleotide, regardless of the method used for insertion. In some embodiments, the effector cell is modified to comprise a non-naturally occurring nucleic acid molecule that is created or modified by the hand of man (e.g., using recombinant deoxyribonucleic acid (DNA) technology) or is derived from such a molecule (e.g., by transcription, translation, etc.). An effector cell that contains an exogenous, recombinant, synthetic, and/or otherwise modified polynucleotide is considered to be a genetically engineered effector cell.

“Nucleic acid”, as used herein, includes and/or refers to a “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In some embodiments, the nucleic acid does not comprise any insertions, deletions, inversions, and/or substitutions. However, it may be suitable in some instances, as discussed herein, for the nucleic acid to comprise one or more insertions, deletions, inversions, and/or substitutions. In some embodiments, the nucleic acid can encode additional amino acid sequences that do not affect the function of the CAR and polynucleotide and which may or may not be translated upon expression of the nucleic acid by a host cell.

Nucleic acids can be obtained using any suitable method, including those described by Maniatis et al., Molecular Cloning: A Laboratory Manual, Cold Spring Harbor, N.Y., pp. 280-281 (1982) and/or U.S. Publication No. US2002/0190663, each of which are herein incorporated in their entireties for their teachings. Nucleic acids obtained from biological samples typically are fragmented to produce suitable fragments for analysis.

Nucleic acids and/or other moieties can be isolated. As used herein, “isolated” includes and/or refers to separate from at least some of the components with which it is usually associated whether it is derived from a naturally occurring source or made synthetically, in whole or in part. Nucleic acids and/or other moieties of the invention can be purified. As used herein, “purified” includes and/or refers s separate from the majority of other compounds or entities. A compound or moiety can be partially purified or substantially purified. Purity can be denoted by a weight by weight measure and can be determined using a variety of analytical techniques such as but not limited to mass spectrometry, HPLC, etc.

### Experimental Embodiments

A number of experimental embodiments were conducted to generate genetically engineered effector cells and to characterize the effector cells functionality. Additional experiments were conducted to generate an optimized process for forming genetically engineered effector cells for in vivo synthesis of engineered proteins. As further described below, the process can optimize the transformation (e.g., transduction) yield, the expansion rate, and the functionality of the engineered effector cells. Example constructs used to generate genetically engineered effector cells include the nucleotide sequences set forth in SEQ ID NOs: 1-20. SEQ ID NOs: 1-20 are each synthetic DNA.

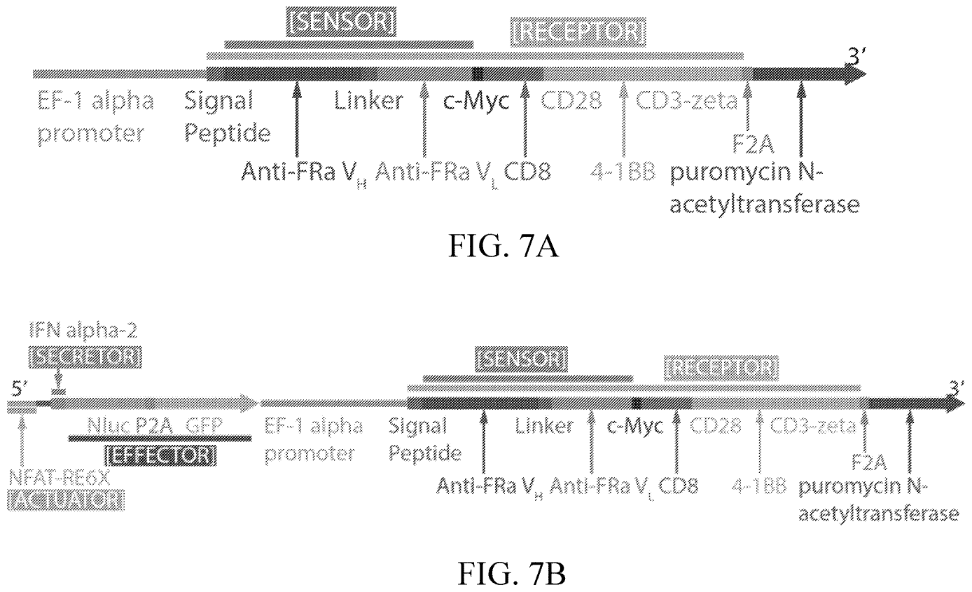

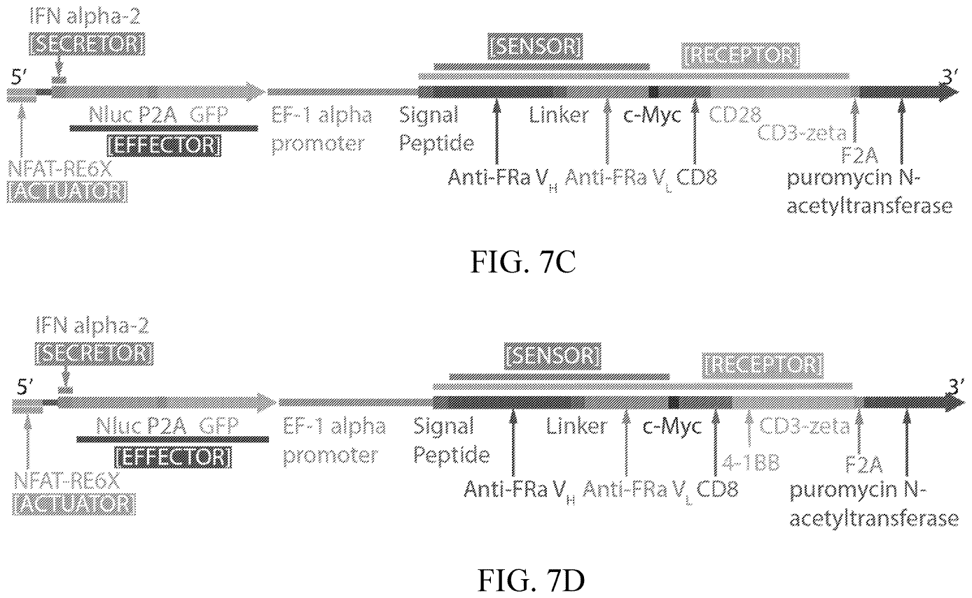

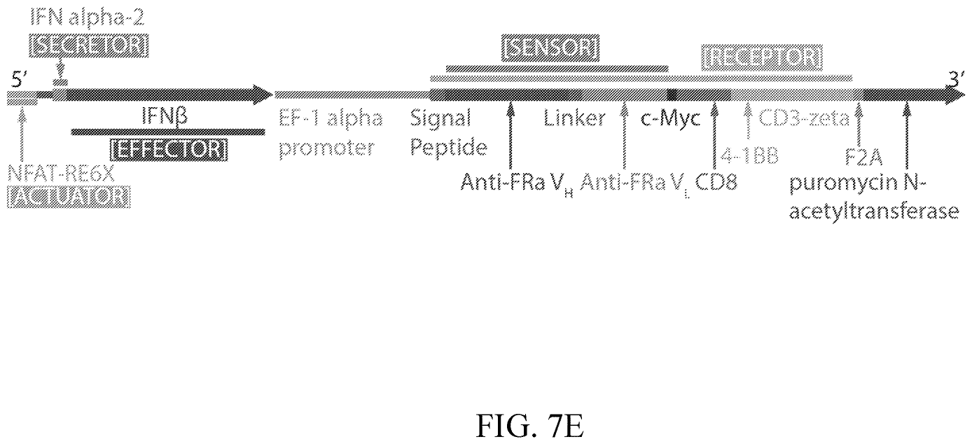

FIGS. 7A-7E illustrate example polynucleotide sequences used to form genetically engineered effector cells, in accordance with the present disclosure. FIG. 7A illustrates an example polynucleotide sequence (SEQ ID NO: 1) that includes a receptor element encoding a CAR, with the CAR including an antigen binding domain (e.g., against Folate Receptor alpha (FRα)), a transmembrane (e.g., CD8), and an intracellular signal domain of CD28, 4-1BB and CD3 zeta (SEQ ID NO: 3). FIG. 7B illustrates an example polynucleotide sequence (SEQ ID NO: 2) that includes the receptor element of FIG. 7A, an actuator element (e.g., NFAT-RE 6×), and an effector element (e.g., Nluc-P2A-GFP, SEQ ID NO: 19). FIG. 7C illustrates an example polynucleotide sequence (SEQ ID NO: 4) that includes the actuator element (e.g., NFAT-RE 6×) and an effector element (e.g., Nluc-P2A-GFP) of FIG. 7B, with a receptor element encoding a CAR, the CAR including an antigen binding domain (e.g., against FRα), a transmembrane (e.g., CD8), and an intracellular signal domain of CD28 and CD3 zeta (SEQ ID NO: 5). FIG. 7D illustrates an example polynucleotide sequence (SEQ ID NO. 6) that includes the actuator element (e.g., NFAT-RE 6×) and effector element (e.g., Nluc-P2A-GFP) of FIG. 7B, with a receptor element encoding a CAR, with the CAR including an antigen binding domain (e.g., against FRα) a transmembrane (e.g., CD8), and an intracellular signal domain of 4-1BB and CD3 zeta (SEQ ID NO: 7 or SEQ ID NO: 20). FIG. 7E illustrates an example polynucleotide sequence (SEQ ID NO: 8) that includes a receptor element encoding a CAR, with the CAR including an antigen binding domain (e.g., against FRα), a transmembrane (e.g., CD8), and an intracellular signal domain of 4-1BB and CD3 zeta (SEQ ID NO: 7), an actuator element (e.g., NFAT-RE 6×), and an effector element of IFNβ (SEQ ID NO: 9).

In some experiments, primary CD4 T-cells were engineered, e.g., transformed, into a zero-order cell-based effector cell capable of synthesizing engineered proteins at the disease site, and proportionate to the disease burden over extended periods of time. The CD4 T-cell lines have long persistence and propensities for higher transduction, faster expansion, and more productive transcriptional machinery. Experimental results showed that CD4 T-cells modified with a CAR using the 4-1BB intracellular domain and without the CD28 intracellular domain transduce approximately three times better as compared to CD8 T-cells. These effector cells, formed with the CD4 T-cells, exhibited approximately two-fold expansion rates, produced five times more engineered protein, and displayed minimum cytolytic activity. Specifically, when engineered to induce IFN-β upon interaction with the antigen-presenting target cells, the CD4 T-cell-based effector cell surpassed CD8 T-cells (e.g., produced more anti-tumor IFN-β than produced by CD8 T-cells) effectively suppressing ovarian cells grown in vitro and in vivo.

This technology allows for precise targeting of therapeutic biologics to disease sites while minimizing bioavailability in healthy tissues. Leveraging CD4 T-cells' extended persistence offers the potential to improve patient compliance and enhance disease management by reducing the frequency of drug administration and for human treatment.

More particularly, the CD4 T-cells, compared to its CD8 counterpart, contain an enhanced capacity for transporting specific proteins to the disease site. As noted above, through incorporation of a CAR with 4-1BB intracellular domain, while excluding the CD28 intracellular domain, the CD4 T-cells exhibited a three-fold improvement in transduction efficiency, doubling the rate of expansion, and a five-fold increase in the expression of target proteins. In some experiments, the efficacy of the CD4 T-cell-based delivery system was verified using a CAR that recognized (e.g., bind to) FRα as an antigenic target, prompting the effector cell to produce a bioluminescent reporter enzyme. The modular nature of the CAR allows for the redirection of the specificity of the platform to identify another antigen and induce the expression of a desired clinically relevant therapeutic protein. In further experiments, the CD4 T-cell-based effector cell was modified and further validated for delivering functional therapeutics, e.g., used to deliver interferon-β (IFNβ) and to exert anti-tumor effects in vivo in intraperitoneally (i.p.) implanted ovarian cancer cells.

Various experiments were directed to assessing the phenotype of T-cell used in for the effector cell and the influence on therapeutic outcome, such as by affecting both the efficient production of a dosage suitable for clinical use and the synthesis of effector proteins produced from the cell within the targeted location. More specifically, the dynamics of CD4 and CD8 T-cells were assessed in vitro for transduction, expansion, and the antigen-induced delivery function.

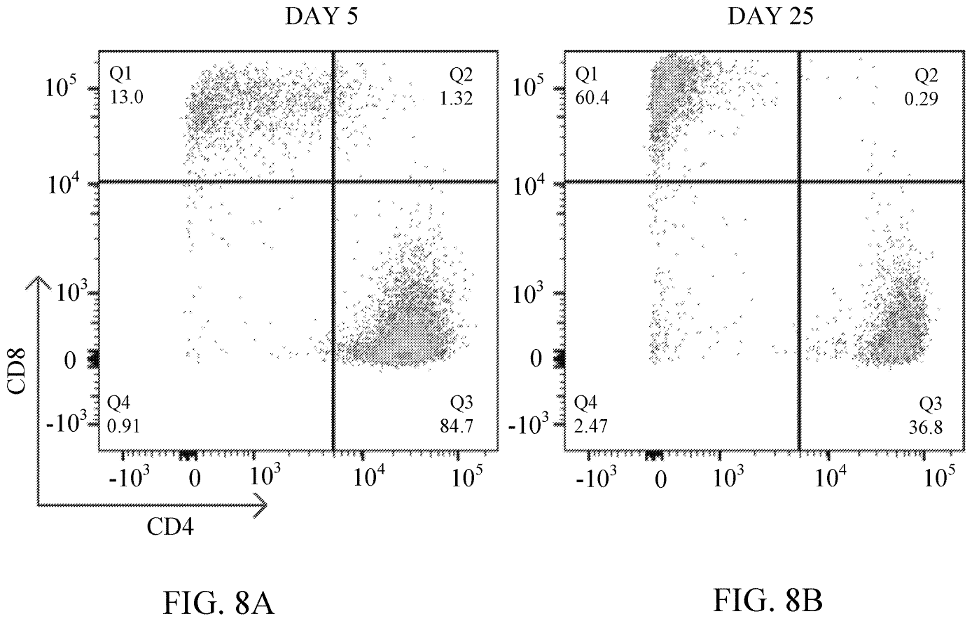

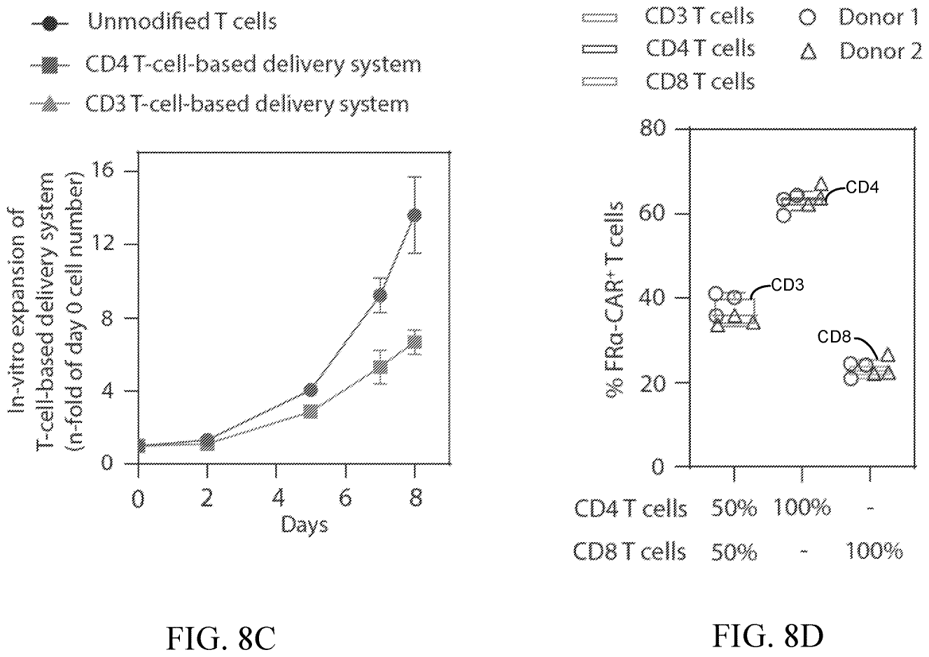

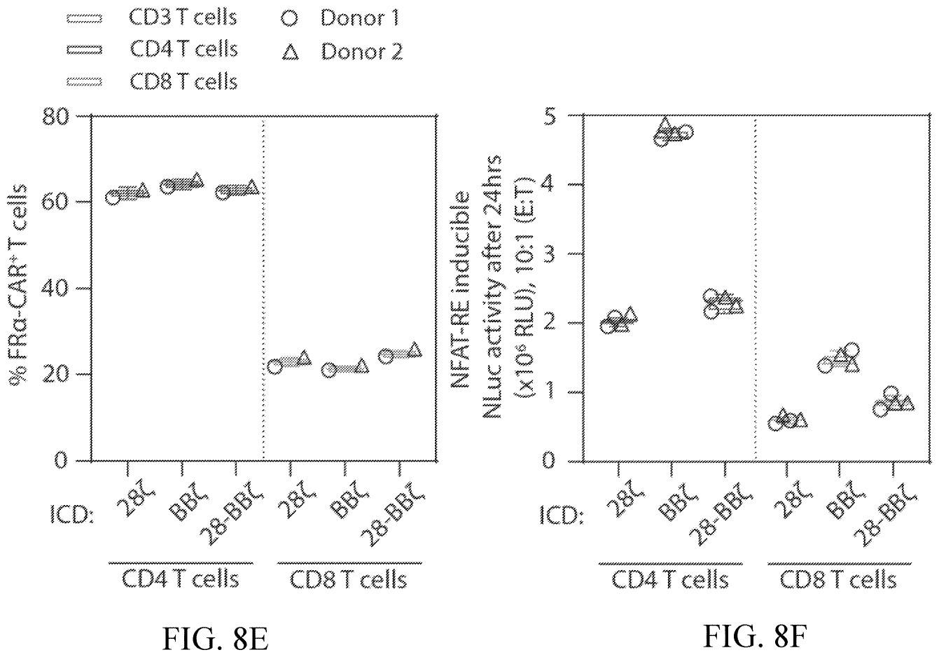

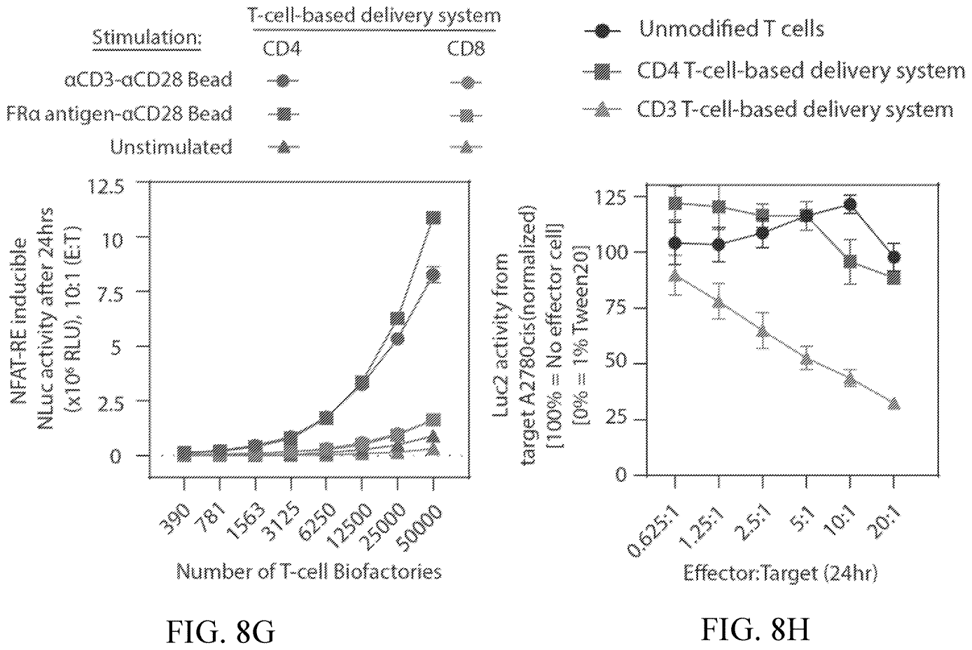

FIGS. 8A-8H illustrate the effects of varying different factors on production and function of the T-cell based effector cell, in accordance with the present disclosure.

Although the ratio of CD4 to CD8 among primary CD3 T-cells that were freshly isolated from the peripheral blood strongly favored CD4 T-cells (CD4: CD8=7:1), the final ratio of the two compartments after transducing and expanding CD3 T-cells for 25 days shifted in favor of CD8 T-cells (CD4: CD8=1:2) (representative data shown in FIGS. 8A-8B). While this may not be critical when forming CAR T-cells where the goal is to maximize the production of cytolytic CD8 T-cells, starting from a combined pool of CD4 and CD8 T-cells may not be the best strategy when engineering effector cells for a delivery function. This is because, as noted above, the impaired NFAT pathway in CD8 T-cells may limit the antigen-induced ability of T-cell to express the engineered protein. FIGS. 8A-8B show the results of CD3 T-cells engineered for delivery function when expanded for 25 days and assessed for distribution of CD4 and CD8 phenotypes on day 5 and day 25.

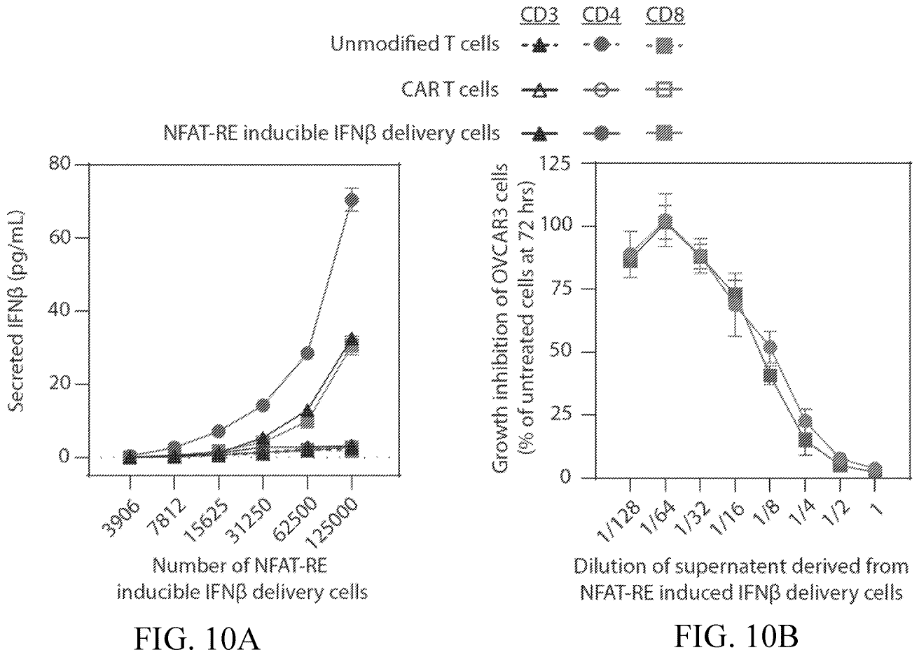

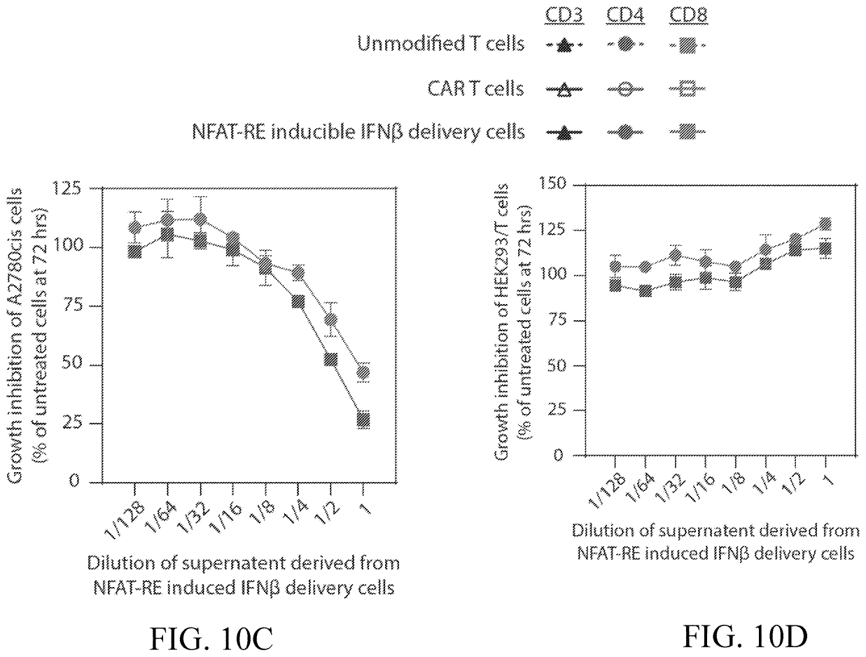

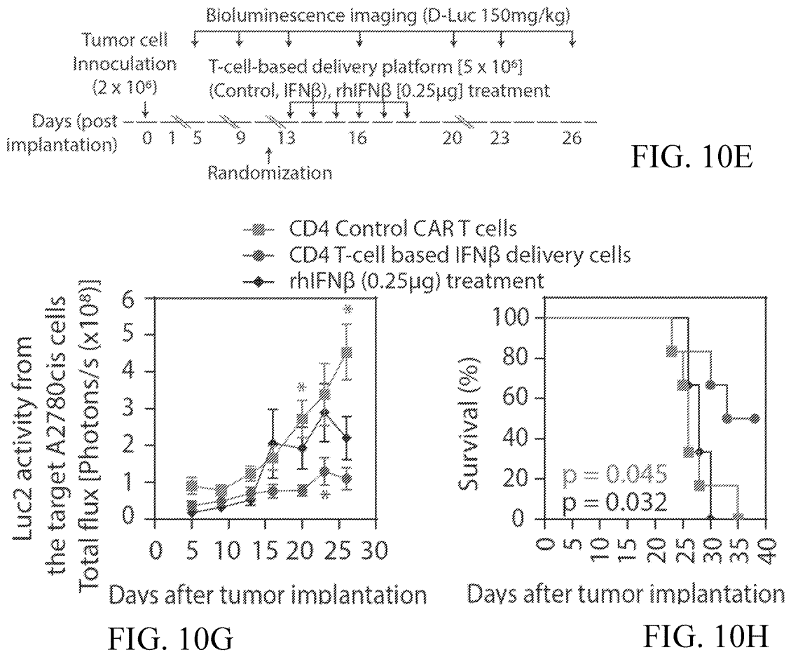

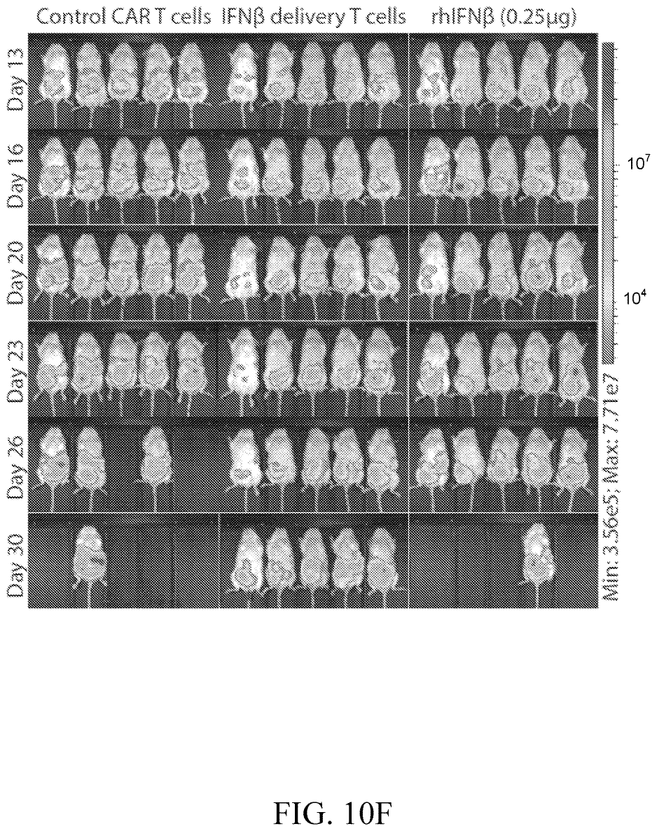

The shift toward the CD8 phenotype during the expansion was observed when the T-cell culture contained CD8 and CD4 T-cells. FIG. 8C shows results of donor-matched CD4 T-cells and CD3 T-cells (contains CD4 and CD8) that were numerically expanded after engineering for the delivery function. Statistical analysis was performed using Multiple comparison t-test using Holm-Sidak method, ** p<0.01. For example, FIG. 8C shows an around fourteen-fold expansion of the primary CD4 T-cell culture 8 days after transduction, compared to around seven-fold expansion when starting with the CD3 T-cell population (CD4: CD8=7:1 on day 0) from the same donor (p<0.01, at all points beyond day 0). The CD4: CD8 ratio in the CD3 T-cell population on day 0 was around 7:1, and based on data (representative data in FIG. 10), exhibited minimal shift until day 10.

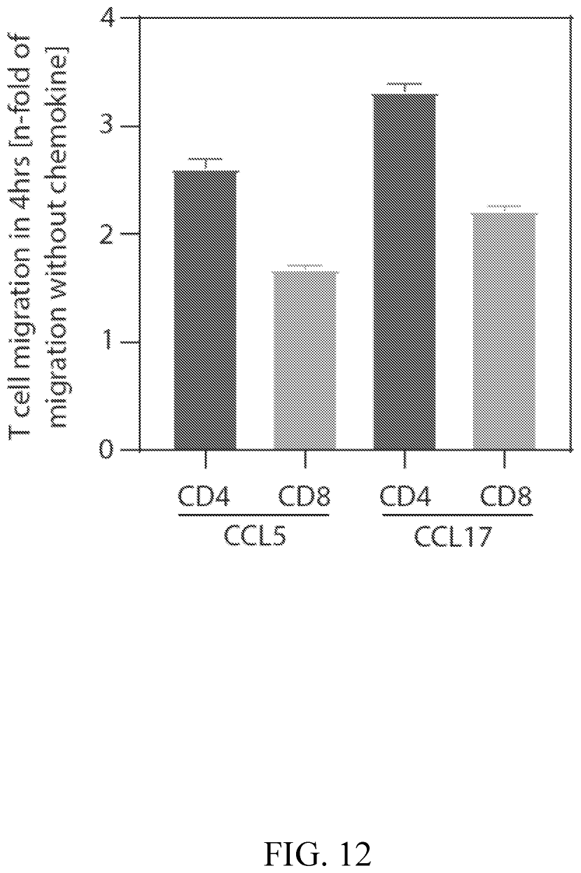

FIGS. 8D-8E show the transduction efficiency of T-cells assessed by measuring FRα-CAR expression with flow cytometry 5 days after transduction. The results on the comparison of transduction efficiencies of CD4 and CD8 phenotypes further support use the CD4 phenotype for the delivery function. The CD3 population was reconstructed by combining CD4 and CD8 T-cells at a 1:1 ratio. More specifically, FIG. 8D shows donor-matched CD4 (100%), CD8 (100%), CD4 and CD8 T-cells (50% each) when engineered for delivery function (n=2 healthy donors, assayed in triplicate). To account for the variance due to human genetic diversity, the experiment was repeated three times on different days with two healthy human donors (represented by different symbols). All transductions were performed using the same process and the efficiency of transduction was determined after 5 days by assessing the percentage of engineered T-cells. Compared to the CD8 T-cells, the CD4 T-cells not only expanded faster (around two-fold, FIG. 8C) but also transduced more easily (around three-fold, FIG. 8D). It was also observed that when compared to the CD8 T-cells, CD4 T-cells exhibited a stronger tendency to chemotactically migrate toward chemokines, CCL5 and CCL17 (FIG. 12), when enriched in the tumor microenvironment. This finding is in consensus with reports, where antigen-reactive CD4 T-cells are reported to efficiently infiltrate into immunologically cold tumors and render the tumor microenvironment receptive to cytotoxic CD8 T-cells resulting in tumor rejection.

The experiments in FIGS. 8A-8D used a CAR that included the intracellular domains of CD28 and 4-1BB in addition to the CD3 zeta domain, as shown by the sequence of FIG. 7A. In further experiments, three combinations of the intracellular CAR domains were assessed for improving performance of CD4 T-cells to serve as a delivery platform. The results are shown in FIGS. 8E and 8F. The three CAR constructs included intracellular domains from (i) CD28 only (e.g., FIG. 7C) (285), (ii) 4-1BB only (BBC) (e.g., FIG. 7D), and (iii) CD28 and 4-1BB in tandem (28-BB) (e.g., FIG. 7B). Complete schematics of the individual CAR constructs are shown in FIGS. 7A-7D. FIGS. 8E-8F shows the results, which indicate that, while transduction efficiency (FIG. 8E) of the CD4 T-cell is independent of the combination of the intracellular domains used in the CAR, the expression level of the engineered protein (represented by a bioluminescent reporter enzyme, FIG. 8F), depends on these domains. The results showed that the BBC CAR, e.g., the CAR that included the 4-1BB intracellular domain but not the CD28 intracellular domain of FIG. 7D, was more effective in inducing the engineered reporter enzyme in both CD4 and CD8 T-cell-based effector cells. The other two CARs (e.g., 285 and 28-BB of FIGS. 7B-7C) induced the reporter activity to a similar extent. However, the reporter enzyme activity induced by the BBL CAR was around five-fold more in the CD4 T-cell based effector cell compared to that in the CD8 T-cell based effector and was specific in response to the antigen-presenting target cell (FRα+OVCAR3 cell, in this case). Further compared to the 28ζ CAR, the BB imparts other traits in the CAR T-cells, such as increased persistence, reduced tonic signaling, and better toleration by patients in terms of cytokine release syndrome and cell therapy-associated neurotoxicity.

More specifically, FIG. 8E shows donor-matched CD4 (100%) and CD8 (100%) T-cells engineered for the delivery function using CARs with different intracellular domains (28ζ, BBζ, and 28-BBζ) (n=2 healthy donors) and as respectively illustrated by FIGS. 7B-7D. As used herein, intracellular domains of 28ζ includes intracellular domains of CD28 and CD3-zeta (and genetically engineered effector cells were generated by transducing T-cells using the sequence as shown by FIG. 7C), intracellular domains of BBC includes 4-1BB and CD3-zeta (and genetically engineered effector cells were generated by transducing T-cells using the sequence as shown by FIG. 7D), and intracellular domains of 28-BB ζ includes CD28, 4-1BB, and CD3-zeta (and genetically engineered effector cells were generated by transducing T-cells using the sequence as shown by FIG. 7B). The results in FIG. 8E show that the process yield, when transducing CD4 T-cells was around three-fold higher than the CD8 T-cells and around 1.5-fold higher than the representative CD3 population (CD4: CD8=1:1).

FIG. 8F shows donor-matched CD4 (100%) and CD8 (100%) T-cells engineered for the delivery function using CARs with different intracellular domains (28ζ, BB ζ, and 28-BBζ) (n=2 healthy donors, assayed in triplicate) and co-cultured with OVCAR3 target cells (E:T=10:1). The activity of the NFAT-RE-induced reporter (Nluc) was quantified to evaluate the impact of different intracellular domains on the delivery function of both CD4 and CD8 T-cell phenotype. For FIGS. 8D-8F, the statistical analysis and p values were determined by one-way ANOVA and Tukey's multiple comparison test, *p<0.05, **** p<0.0001.

Further experiments were directed to assessing whether the reduced expression of an engineered effector protein, when using NFAT-RE transcriptional start sites, in the CD8 T-cell-based effector cell was due to the impaired NFAT-based transcriptional machinery. Alternatively, this may have resulted from the reduced antigenic stimulation due to the cytolytic action of the FRα-specific CD8 T-cell delivery platform against the antigen-presenting target cells. To assess this, the FRα-presenting target OVCAR3 cells were placed with microparticles (2.8 μm diameter) functionalized with human FRα antigen and anti-human CD28 antibodies. Similar microparticles functionalized with anti-CD3 and anti-CD28 antibodies served as the positive control, and unstimulated CD4 and CD8 T-cells (engineered for delivery function) were used as negative controls. The results shown in FIG. 8G show around ten-fold higher activity (e.g., higher expression) of the bioluminescent reporter, validating that the CD4 T-cell-based effector cell has the stronger NFAT-based transcriptional machinery for a robust engineered function, which is impaired in the CD8 T-cells and is not due to the reduction in the number of antigen-presenting target cells. More particularly, FIG. 8G shows a comparison of Nluc activity in CD4 and CD8 T-cells engineered for the delivery function (with BB(CAR) when stimulated by microparticles. Microparticles were functionalized with anti-CD3 and anti-CD28 antibodies or with FRα antigen and anti-CD28 antibodies. Unstimulated CD4 and CD8 T-cells were used as negative controls. The Nluc activity is represented as a function of increasing number of engineered CD4 and CD8 T-cells where cell-to-particle ratio (represented as E:T) is constant (10:1). For FIG. 8G, the statistical analysis and p values were determined by 2-way ANOVA and Tukey's multiple comparison test, ** p<0.01.

FIG. 8H shows the cytolytic function of donor-matched FRα-CAR CD4 and FRα-CAR CD3 T-cells (engineered for delivery function) against FRα+ tumor cells (A2780cis-FRα+Luc2+). Unmodified CD3 T-cells were used as negative control. Statistical analysis p values were determined by multiple comparison t-test using the Holm-Sidak method, *** p<0.001. All results are represented as mean±SD. The data shown in FIG. 8H verifies that the CD4 T-cells engineered for delivery function exhibit minimum cytolytic activity. Compared to the CD8 T-cells, CD4 T-cell-based effector cells can be administered at an increased dose for a higher maximum recommended starting dose in the first-in-human clinical trials. In the experiment, the CD4 and CD8 T-cells, both engineered for FRα specificity, were incubated with target cells engineered to present FRα (e.g., FRα+A2780cis cells, as described in C. E. Repellin et al., Engineered Ovarian Cancer Cell Lines for Validation of CAR T-Cell Function, Advanced Biosystems 4, 1900224 (2020), which is incorporated herein by reference in its entirety for its teaching). The target cells were also engineered to express the Luc2® enzyme, an ATP-dependent bioluminescent reporter, that served as a live-cell marker. Non-engineered primary CD3 T-cells were used as a negative control. The results confirmed that the significantly low cytolytic activity in the CD4 T-cell-based effector cells compared to the CD3 T-cell-based effector cells (CD4: CD8=1.5:1) (p<0.01, at all E:T), and attests to selection of the CD4 T-cell as the suitable phenotype for use in a cell-based delivery system. The engineered CD4 T-cells can therefore be delivered at an increased tolerated dose to robustly express the desired protein without exhibiting undesired side effect of killing healthy cells that may be expressing basal levels of the target antigen.

Various experiments were directed to verifying the function of the CD4 T-cell-based effector cells as a protein delivery platform in vivo. For such experiments, immune-deficient mice with i.p. tumors were treated with the CD4 T-cells-based effector cells which were engineered for delivery function (e.g., delivering a protein).

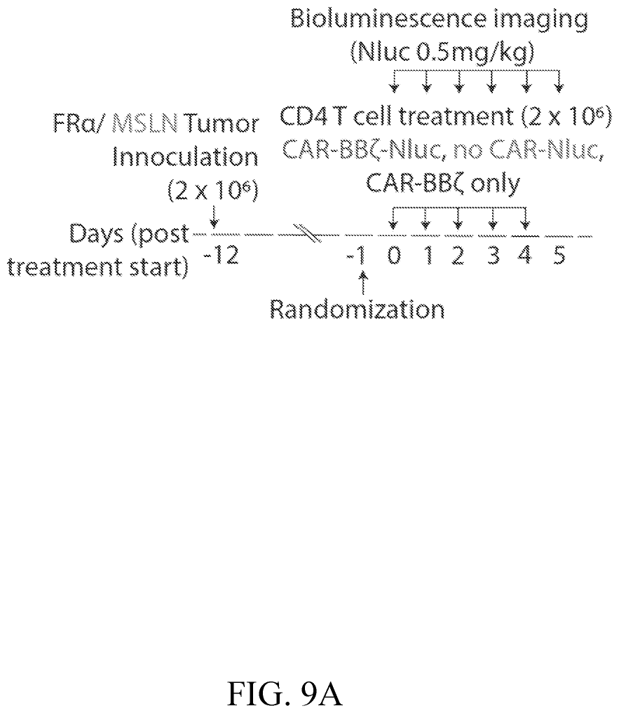

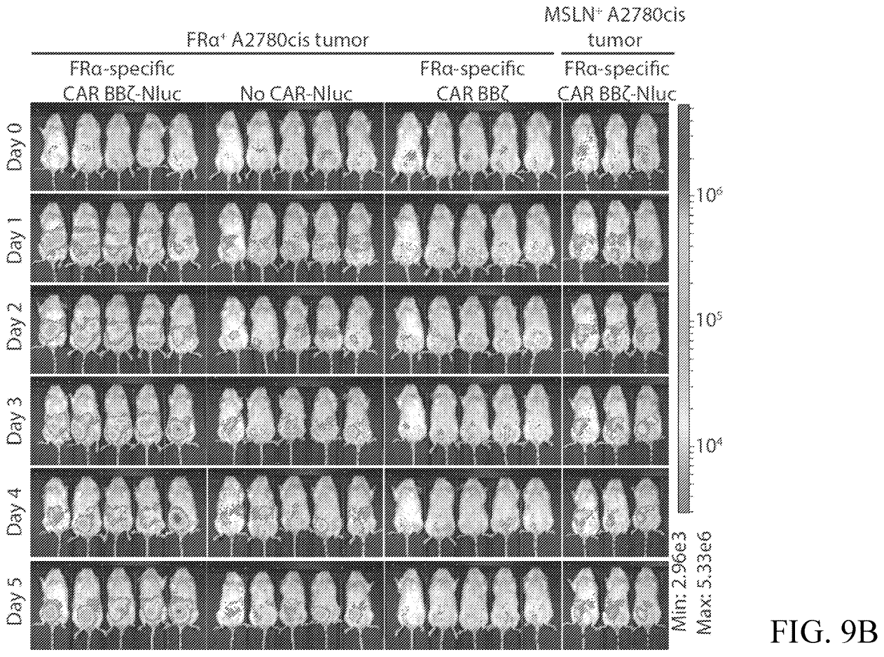

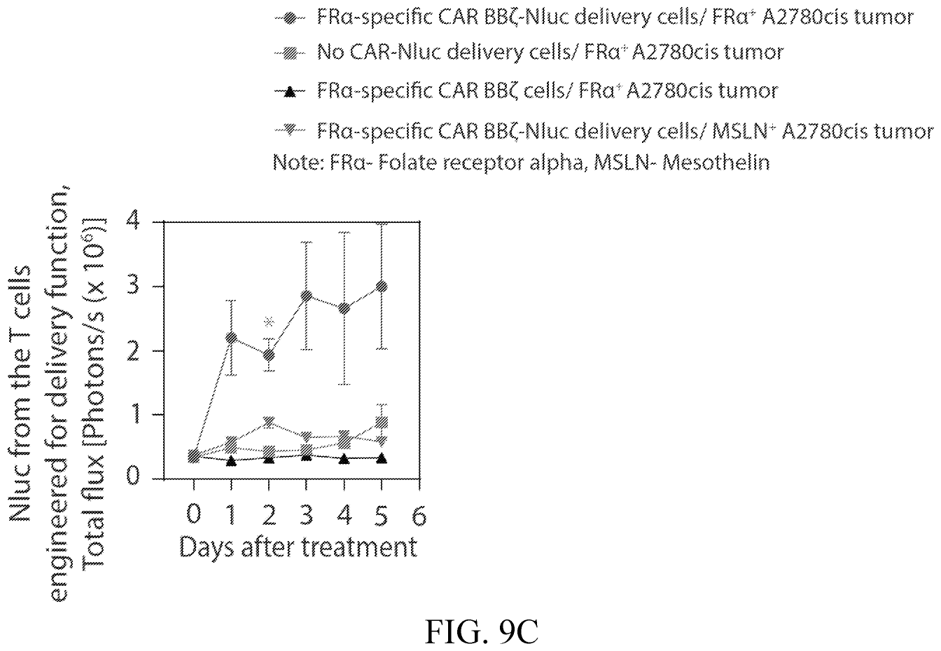

FIGS. 9A-9C illustrate the results of verifying the functionality the CD4 T-cell-based effector cells as a protein delivery platform in vivo, in accordance with various embodiments. The experiment schedule is detailed in FIG. 9A and the results are shown in FIGS. 9B-9C. The 12-day old xenograft tumors (antigen positive FRα+MSLN^{neg} A2780cis, antigen negative FRα^{neg}MSLN+A2780cis) were treated with 2×10⁶ CD4 T-cell engineered for in situ delivery (CAR-BB ζ-Nluc) on days 0, 1, 2, 3, and 4. The target specific delivery function, e.g., Nluc activity (FIG. 9B) was imaged and quantified (FIG. 9C) at baseline (day 0) as well as on days 1, 2, 3, 4, and 5. The engineered CD4 T-cells, with specificity for FRα antigen, exhibited the delivery function by Nluc reporter activity when stimulated by FRα+ tumors, compared to when stimulated by non-target cells, e.g., FRα^{neg} tumors, confirming the target-specific in situ delivery function. Negative controls included CD4 T-cells with the same inducible delivery function but without a CAR (e.g., no CAR-Nluc) and FRα-specific CD4 CAR (CAR-BBζ) T-cells without the NFAT-RE inducible delivery function. An increase in the delivery, as indicated by the increase in inducible reporter activity, was observed in the Nluc producing FRα-CAR⁺ T-cells, compared to the control groups (FIG. 9B and FIG. 9C). The results indicate use of the CD4 T-cell platform with reporter expression for in vivo sensing. The technology may be used for monitoring disease progression, assessing therapeutic response, and delivering therapeutic proteins in situ.

As shown by FIGS. 9A-9C, the engineered primary CD4 T-cell-based effector cell showed antigen-specific delivery function in vivo (n=6 mice per group). FRα-specific primary T-cells engineered for the NFAT-RE inducible delivery function were i.p. injected in i.p. FRα⁺ MSLN^{neg}A2780cis or FRα^{neg}MSLN⁺A2780cis tumor-bearing NSG mice at 24-hour interval for 5 days and NFAT-RE inducible effector (Nluc) activity was measured for 6 days including the day of injection as a baseline to assess the delivery function. FRα⁺ MSLN^{neg} A2780cis tumors treated with CD4 T-cells engineered without CAR but with NFAT-RE inducible effector (Nluc) or with FRα-specific primary CAR T-cells (engineered without the NFAT-RE inducible effector (Nluc)) served as control groups. FIG. 9A is a schematic of dosing, treatment, and imaging schedules, FIG. 9B includes representative bioluminescent images, and FIG. 9C show quantification results. All results are represented as mean±SEM. Statistical analysis and p values were determined by 2-way ANOVA and Tukey's multiple comparison test, *p<0.05.

Further experiments were directed to exchanging the nucleotide sequence of the bioluminescent reporter enzyme with an antitumor cytokine, interferon-beta (IFNβ) (FIGS. 10A-10D and using the polynucleotide sequence of FIG. 7E). Using microparticles functionalized with FRα antigen and anti-CD28 antibodies, experiments reconfirmed the use of CD4 T-cell for synthesizing engineered therapeutic proteins via the NFAT transcriptional machinery (FIG. 10A). Compared to pan CD3 T-cells and killer CD8 T-cells, engineered for the delivery function, the helper CD4 T-cell-based delivery platform produced two-fold more IFNβ, confirming its enhanced antigen-specific delivery function. The secretion of IFNβ from the respective unstimulated CD3, CD4, and CD8 T-cells (engineered for IFNβ delivery) was minimal and is shown in FIG. 10A. The results in FIGS. 10B-10D show a dose-dependent growth-inhibitory effect of IFNβ on various cell lines (OVCAR3, A2780cis, and HEK293T/17), when the cell lines were treated with the supernatant of CD4 and CD8 T-cells engineered to produce IFNβ. While the growth-inhibitory effects were observed on OVCAR3 (FIG. 10B) and A2780cis (FIG. 10C), this effect was not observed on HEK293/T17 cells (FIG. 10D).

More specifically, FIGS. 10A-10H illustrate FRα-specific targeting of tumor cells by a CD4 T-cell engineered to secrete IFNβ, in accordance with the present disclosure. FIG. 10A shows IFNβ secretion from T-cells (CD4, CD8 and CD3) engineered for delivery function upon stimulation by FRα-antigen/anti-CD28 Dynabeads™ (cell-to-particle ratio of 1:3). For FIG. 10A, statistical analysis and p values were determined by 2-way ANOVA and Tukey's multiple comparison test, ** p<0.01. FIGS. 10B-10D show the growth inhibitory effect of the secreted IFNβ from CD4 and CD8 T-cell-based effector cells as was assessed on OVACR3 (FIG. 10B), A2780cis (FIG. 10C), and HEK293T/17 (FIG. 10D) cell lines. Cells were cultured for 3 days in the presence of serially diluted supernatants obtained from the engineered CD4 and CD8 T-cells stimulated by FRα-antigen/anti-CD28 Dynabeads™. All results are represented as mean±SD. For FIGS. 10B-10D, statistical analysis and p values were determined by multiple comparison t-test using Holm-Sidak method, ** p<0.01.

As shown by FIGS. 10E-10H, the primary CD4 T-cell-based IFNβ effector cell showed tumor growth inhibition in vivo and increased survival of mice (n=6 mice per group). FRα-specific T-cells engineered for NFAT-RE induced IFNβ delivery were i.p. injected (5×10⁶ cells/dose) in i.p. FRα⁺Luc2+A2780cis tumor-bearing NSG mice at 24-hr interval for 6 days and tumor luminescence was measured every 3-4 days to assess tumor growth. The i.p. administration of FRα-specific primary CAR T-cells (engineered without the NFAT-RE induced IFNβ delivery function) or rhIFNβ (0.25 μg in 100 μL) served as control groups. FIG. 10E is a schematic of dosing, treatment, and imaging schedules, FIG. 10F includes representative bioluminescent images, and FIG. 10G shows a quantification of tumor bioluminescence. All results are represented as mean±SEM. Statistical analysis and p values were determined by 2-way ANOVA and Tukey's multiple comparison test. * p<0.05. FIG. 10H show percentage survival of mice in each group (n=6). Statistical analysis was performed by log-rank Mantel-Cox test.

For a comparison of IFNβ dose delivered by the CD4 T-cells to directly administered rhIFNβ dose, calculations were performed on the last point on the x-axis of FIG. 10A. When 125,000 CD4 T-cells engineered to produce IFNβ were stimulated at 1:3 cell-to-particle ratio for 72 hours, the engineered cells produced IFNβ. The rhIFNβ standard was used for calculating the equivalent IFNβ activity in the culture supernatant that, as indicated on the y-axis, was determined to be 70.436 picogram (pg)/mL. Therefore, total amount of IFNβ in the total supernatant volume of 300 μL should be 70.436 pg/mL×300 μL=21 μg IFNβ. Because 5×10⁶ IFNβ-producing CD4 T-cells were injected per dose in each mouse, which is forty times the number of CD4 T-cells used to secrete 21 μg IFNβ (5×106/125,000=40), it was estimated that the effector cells may be secreting in vivo approximately 0.84 ng of IFNβ (21 μg×40=840 pg=0.84 ng). The amount of rhIFNβ injected per dose per mouse was 0.25 μg=250 ng. This is around 300-fold (e.g., 250 ng/0.84 ng=297.62) more than the estimated amount of IFNβ delivered by the CD4 T-cells.

More specifically, FIGS. 10E-10H demonstrate the therapeutic utility of the IFNβ producing CD4 T-cell-based effector cell. To demonstrate the therapeutic utility in vivo, the IFNβ producing CD4 T-cells were used to challenge antigen positive (FRα⁺Luc2+A2780cis) tumors. The experiment schedule is detailed in FIG. 10E and the results are shown in FIGS. 10F-10H. FRα⁺Luc2+A2780cis cells (2×10⁶) were i.p. implanted in NSG mice. The 12-day-old xenograft tumors were i.p. treated with 5×10⁶ FRα-CAR⁺ T-cells (with NFAT-RE inducible IFNβ) daily for 6 days (day 13-day 19). The therapeutic efficacy was assessed by imaging (FIG. 10F) and quantitatively comparing the tumor luminescence (FIG. 10G) with control groups (e.g., 5×10⁶ FRα-CAR⁺ T-cells without NFAT-RE inducible IFNβ; 0.25 μg recombinant human IFNβ (rhIFNβ)). A significant reduction in the tumor mass, as indicated by the reduction in luminescence, was observed in the IFNβ producing FRα-CAR⁺ T-cells, compared to the control FRα-CAR⁺ T-cells that did not produce IFNβ, indicating that the CD4 T-cell-based effector cells produce functional therapeutics (FIG. 10G). The results in FIG. 10H show statistically significantly improved survival in mice treated with IFNβ producing FRα-CAR⁺ T-cells, when compared to the direct injection of rhIFNβ and control FRα-CAR⁺ T-cells that did not produce IFNβ. The rhIFNβ treatment did not show any survival advantage and all mice were sacrificed as a result of weight loss or distress.

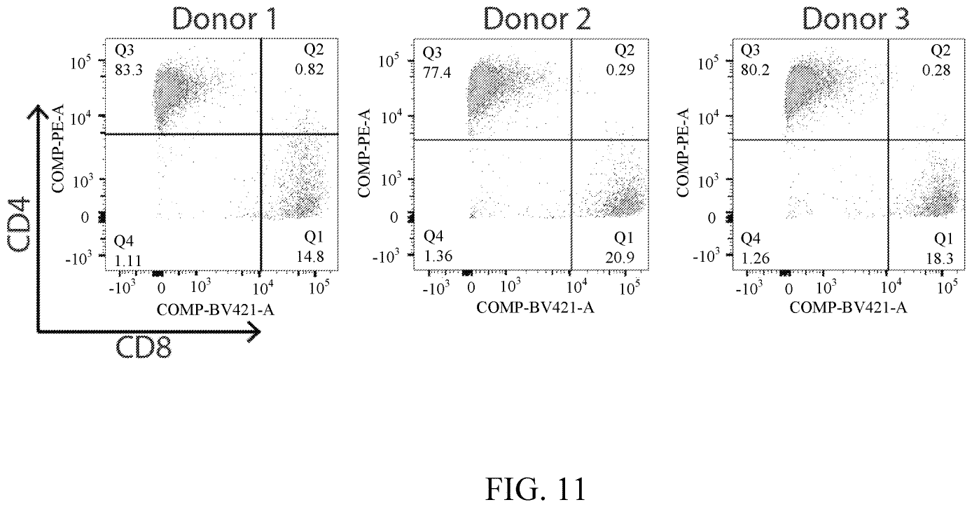

FIG. 11 illustrates flow cytometry plots showing the proportion of CD4 and CD8 T-cells in the pan CD3 T-cell population from healthy donors (e.g., 3 healthy donors at day 10 of in vitro expansion), in accordance with the present disclosure.

FIG. 12 illustrates a comparison of CD4 and CD8 T-cell chemotaxis, in accordance with the present disclosure. CD4 and CD8 T-cells were compared for their migration toward chemotactic gradients of CCL5 and CCL17. Statistical analysis was performed by two-tailed Student's t-test, **** p<0.0001.

The following describes the chemokine induced T-cell migration assay used in FIG. 12. A Boyden chamber Transwell® migration assay was performed to assess chemokine induced migration. Donor-matched CD4 and CD8 T-cells were thawed and stimulated with anti-CD3/CD28 Dynabeads™ (at 1:3 cell: particle ratio). Three days after stimulation, the cells were de-beaded and serum starved overnight by keeping the cells in 2% heat inactivated FBS containing media. Transwell® permeable inserts with a pore size of 5 μM were used for the assay and were pre-soaked in serum-free RPMI for 30 minutes in a cell culture incubator (37 degrees C., 5% CO₂, 95% humidity). The serum starved CD4 and CD8 T-cells were counted and 5×10⁵ cells per insert were resuspended in 100 μL serum-free RPMI and seeded on to the top chamber of the insert. 650 μL of complete growth medium supplemented with 25 nanograms (ng)/mL CCL5 or 25 ng/ml CCL17 was added to the bottom chamber to serve as chemoattractant. T-cell migration was performed for 4 hours in a cell culture incubator (37 degrees C., 5% CO₂, 95% humidity). After incubation, the insert (top-chamber) was removed and the cells in the bottom chamber were quantified using CellTiter-Glo® reagent. All samples were run in triplicate wells and the data is represented as mean±SD of n-fold migration corresponding to the CD4 or CD8 T-cell migration in absence of chemoattractant.

I. Synthesis and experimental information for CD4 T-cells as chassis for genetically engineered effector cells.
(1) Materials and reagents. Table 1 lists sources for materials, supplies, services, and equipment used in the above-described experiments.

TABLE 1
Resources Table

| | | |
|---|---|---|
| | | IDENTIFIER |
| REAGENT OR RESOURCE | SOURCE | (CAT#) |

Biological samples

| | | |
|---|---|---|
| Human primary CD3 T-cells | Stanford Blood Center | A1020 |
| Human primary CD4 T-cells | Stanford Blood Center | A1015 |
| Human primary CD8 T-cells | Stanford Blood Center | A1016 |
| A2780cis | Sigma-Aldrich | 93112517 |
| OVCAR3 | ATCC | HTB-161 |
| HEK293T/17 | ATCC | CRL-11268 |
| Human HEK293 cells - Type I | InvivoGen | hkb-ifnab |
| IFNs Reporter Cells | | |
| NOD.Cg-Prkdc^{scid} Il2rg^{tm1Wj1}/SzJ | Jackson Laboratory | 005557 |
| (NSG) | | |

Chemicals, peptides, recombinant proteins

| | | |
|---|---|---|
| Fetal bovine serum (FBS) | Sigma-Aldrich | F2442-500 ML |
| Dimethyl sulfoxide, DMSO | Sigma-Aldrich | D2650-100 mL) |
| GlutaMAX ™ | Gibco | 35050-061 |
| Penicillin streptomycin solution | Corning | 30-002-Cl |
| Human IL-2 | Miltenyi Biotec | 130-097-748 |
| Human IL-15 | PeproTech | 200-15-50 UG |
| Human IL-7 | PeproTech | 200-07-50 UG |
| Dynabead Biotin Binder | Invitrogen | 11047 |
| Human CCL5 | PeproTech | 300-06 |
| Human CCL17 | PeproTech | 300-30 |
| DMEM media | Corning | 10-013-CV |
| Transporter 5 ™ | Polysciences, Inc. | 26008-5 |
| RPMI1640 | Corning | 10-040-CV |
| Phosphate buffered saline (PBS) | Corning | 21-040-CV |
| without Ca⁺² and Mg⁺² | | |
| Puromycin dihydrochloride | ThermoFisher Scientific | A1113803 |
| Human IFN-β | R&D systems | 8499-IF/CF |
| Biotinylated human Folate | Acro Biosystems | FO1-H82E2 |
| receptor 1 (FOLR1 -His Tag -Avi | | |
| Tag) | | |
| D-Luciferin | Perkin Elmer | 122799 |
| Acridine Orange Propidium | Nexcelom | CS2-0106-5 mL |
| Iodide (AOPI) staining solution | | |
| Cell-staining buffer | Biolegend | 420201 |
| QUANTI-Blue ™ | InvivoGen | rep-qbs2 |

Antibodies

| | | |
|---|---|---|
| Biotin anti-human CD3 | Biolegend | 317320 |
| Biotin anti-human CD28 | Biolegend | 302904 |
| PerCR/Cy5.5 anti-human CD3 | Biolegend | 300328 |
| antibody | | |
| PE anti-human CD4 antibody | Biolegend | 300508 |
| Brilliant Violet 421 anti-human | Biolegend | 344748 |
| CD8 antibody | | |
| LIVE/DEAD ™ Fixable Aqua | Life Technologies | L34966 |
| Dead Cell Stain Kit | | |
| APC-streptavidin | Biolegend | 405207 |

Plasmids

| | | |
|---|---|---|
| Lentivector transfer plasmid | System Biosciences | CD510B-1 |
| psPAX2 | Addgene | 12260 |
| pAdVAntage | Promega | E1711 |
| pMD2.G | Addgene | 12259 |

Commercial assays

| | | |
|---|---|---|
| Nano-Glo ® assay | Promega | N1120 |
| One-Glo ® assay | Promega | E6110 |
| CellTiter-Glo ® Luminescent Cell | Promega | G7570 |
| Viability Assay | | |

Other materials

| | | |
|---|---|---|
| T150 Flasks | Corning | 430825 |
| Hera 150i CO2 incubator | Thermo Scientific | 50116047 |
| Cellaca slides | Nexcelom | CHT4-PD100- |
| | | 003 |
| 6-well incubation plate | Fisher scientific | FB012927 |
| 96-well plate | Greiner bio-one | 655083 |
| Costar Transwell ® Permeable | Corning | 3421 |
| Supports | | |
| 0.45 μm filter | Corning | 430514 |
| 500 mL collection bottle | Corning | 430282 |
| Konica ultracentrifugation tube | Beckman Coulter | C14291 |
| Nexcelom K2 cellometer | Nexcelom | |
| DynaMag ™-2 sample rack | Life Technologies | 12322D |
| BD FACS Symphony A3 cell | BD Biosciences | |
| analyzer | | |
| EnVision ™ Multilabel Plate | Perkin Elmer | Model 2104- |
| Reader | | 0010A |
| IVIS Lumina X5 imaging system | Perkin Elmer | CLS148590 |
| Optima XPN-90 ultracentrifuge | Beckman Coulter | |

Software

| | | |
|---|---|---|
| FlowJo ® software | FlowJo LLC | |
| Living Image software | Perkin Elmer | 128110 |
| SnapGene software | GSL Biotech LLC | |
| Prism version 8.0 | GraphPad Software | |

(2) Preparations. Transfer plasmids with different genetic payloads were designed in SnapGene software and sub-cloned into the lentivector plasmid. Epoch Life Science, Inc. (Missouri City, TX) provided plasmid preparation services (chemical synthesis of DNA insert sequences, sub-cloning into respective vector backbones, and the amplification). Target cells: FRα⁺OVCAR3 and FRα⁺A2780cis engineered to express modified firefly luciferase (Luc2), as described in (i) C. E. Repellin et al., Modular Antigen-Specific T-cell Biofactories for Calibrated In Vivo Synthesis of Engineered Proteins, Advanced Biosystems 2, 1800210 (2018); and (ii) C. E. Repellin et al., Engineered Ovarian Cancer Cell Lines for Validation of CAR T Cell Function, Advanced Biosystems 4, 1900224 (2020) (each of which are incorporated herein by reference in their entireties for their teachings), were maintained in complete growth media [RPMI1640, 10% heat-inactivated FBS, and 1× penicillin streptomycin solution]. Primary T-cells were maintained in complete growth medium [RPMI1640, 10% heat-inactivated FBS, 1× penicillin streptomycin solution, and 2× GlutaMAX™]. Phosphate buffered saline (PBS) without Ca⁺² and Mg⁺² was used to minimize cell clumping. When applicable, puromycin N-acetyltransferase was used as a selection marker and puromycin dihydrochloride (Puromycin) was used for selecting stable cell lines. Biotinylated human FRα protein was used to analyze FRα CAR expression on engineered primary T-cells.
(3) Method for producing lentivector particles. Lentivector particles were produced as described in H. Radhakrishnan, H. S. Javitz, P. Bhatnagar, Lentivirus Manufacturing Process for Primary T-Cell Biofactory Production, Advanced Biosystems 4, 1900288 (2020), which is incorporated herein by reference in its entirety for its teachings. Briefly, 2^{nd} generation lentivector packaging system was used to prepare lentivector particles. HEK293T/17 producer cells (12×10⁶) were seeded into tissue culture treated T150 flasks in 21 mL complete DMEM supplemented with 10% heat-inactivated FBS and 1× penicillin streptomycin solution and placed in a cell culture incubator (37 degrees C., 5% CO₂, 95% humidity). After 24 hours, transfer plasmid was co-transfected with 2^{nd} generation packaging plasmids (psPAX2, pMD2.G), and pAdV Antage plasmid at 4:3:1:0.4 weight (wt)-ratio, respectively (transfer plasmid: 12 μg, pxPAX2:9 μg, pMD2.G: 3 μg, pAdV: 1.2 μg). Transporter 5TM transfection reagent was used following the manufacturer's protocol (100 μL). Cell culture supernatant enriched with pseudo-viral particle was collected and replenished every 24 hours for 3 days (30 mL). The lentivector-enriched cell culture supernatant was clarified using a 0.45 μm filter. The supernatant was clarified by transferring it to a polypropylene Konical ultracentrifugation tube and centrifuging at 20,700 Gravitation force (G) in an SW32-Ti rotor using a Beckman Coulter Optima XPN-90 ultracentrifuge at 4° C. (C) for 2 hours. The resulting pellet was resuspended in 400 μL serum-free RPMI and aliquoted. An MOI of 10, when the lentivector particles produced in this process, was used to transduce 1×10⁶ cells. The lentivector aliquots were stored at −80 degrees C. until use.
(4) Method for producing engineered primary T-cells-based delivery system. The primary T-cells engineered with NFAT-RE inducible drug delivery system and used in the in vivo validation experiments (FIGS. 9A-9C and FIGS. 10E-10H) were formed using the process described below. Briefly, human primary T-cells (CD3, CD4 or CD8) were purchased from the Stanford Blood Center (Palo Alto, CA). The T-cells were counted and used fresh or were cryostocked using freezing media [90% heat-inactivated fetal bovine serum (FBS) and 10% dimethyl sulfoxide (DMSO)] in liquid nitrogen for future use. Biotin anti-human CD3 and Biotin anti-human CD28 antibody were loaded on Dynabead® Biotin Binder paramagnetic particles following the manufacturer's instructions (anti-CD3/CD28 Dynabeads). Frozen human primary T-cells were thawed (Day 0), resuspended in complete growth medium, and activated by anti-CD3/CD28 Dynabeads (cell: particle of 1:3). After 24 hours (Day 1), 1×10⁶ activated primary T-cells were transduced with the appropriate lentivector particles resuspended in 0.1 mL volume of serum-free RPMI at an MOI of about 10 and in the presence of 8 μg/mL polybrene. The 0.1 mL aliquots of transduction reaction mix were placed as drops in a tissue-culture treated 6-well plate and placed in a cell culture incubator (37 degrees C., 5% CO₂, 95% humidity) for 16 hours. After 16 hours of incubation (around Day 2), the transduced primary T-cells were cultured at 1×10⁶ cells/mL in complete growth medium supplemented with recombinant human IL-7 (25 IU/mL), recombinant human IL-15 (25 IU/mL), and 8 μg/mL polybrene. The cells were counted after another 24 hours (Day 3) and every other day thereafter using acridine orange and propidium iodide (AOPI) staining in a Nexcelom K2 cellometer. They were maintained at a concentration of 0.5×10⁶ cells/mL in complete growth medium supplemented with recombinant human IL-7 (25 IU/mL) and recombinant human IL-15 (25 IU/mL) and the media was replaced every 2-3 days with half media changes. No polybrene was added on Day 3 and beyond.
(5) Flow cytometry analysis. The production yield of engineered primary T-cells was determined by assessing the expression of FRα-CAR on the T-cells engineered for drug delivery (% FRα-CAR⁺ T-cells). Five days after transduction, about 1×10⁶ T-cells were collected and de-beaded by keeping the T-cell suspension on a DynaMag™-2 sample rack for 2 minutes to remove the Dynabead biotin binder particles. The de-beaded T-cells were washed in cell-staining buffer and stained for 1 hour at 4 degrees C. using an antibody cocktail containing biotinylated human FRα protein (FOLR1-His Tag-Avi Tag), PerCR/Cy5.5 anti-human CD3 antibody and the LIVE/DEAD™ Fixable Aqua Dead Cell Stain Kit. The cells were washed, and a secondary staining was performed for 1 hour at 4 degrees C. using APC-streptavidin. The samples were washed, resuspended in 200 μL Cell Staining Buffer, and analyzed with a BD FACS Symphony A3 (BD Biosciences). The data was further processed using FlowJo® software. To determine the proportion of CD4 and CD8 phenotypes in CD3 T-cell culture (FIGS. 8A-8B), a single-staining step protocol was followed whereby the de-beaded T-cells were washed and stained for 1 hour at 4 degrees C. using an antibody cocktail containing PerCR/Cy5.5 anti-human CD3 antibody, brilliant violet 421 anti-human CD8 antibody, PE anti-human CD4 antibody and the LIVE/DEAD™ Fixable Aqua Dead Cell Stain Kit. All samples were analyzed using BD FACS Symphony A3 (BD Biosciences) and the data was further processed using FlowJo® software.
(6) In vitro assessment of delivery function of the engineered T-cells (engineered for delivery function). FRα-CAR⁺CD4 and CD8 T-cells (with NFAT-RE inducible delivery function) were co-cultured with the targets (OVCAR3 or FRα-antigen/anti-CD28 Dynabeads™) at an effector-to-target ratio (E:T) of 10:1, in 200 μL of complete growth medium in a single well of a 96-well plate. After a 24-hours co-culture, the manufacturer's protocol was followed to measure the reporter activity, e.g., NanoLuc (Nluc) activity in the engineered primary T-cells using Nano-Glo® assay. Briefly, the Nluc substrate was diluted in the cell lysis buffer provided with the Nano-Glo® assay and added to the co-culture in 96-well plates to assess the enzyme (Nluc) activity. Following a brief incubation period of 3 minutes, the bioluminescence was read on a microplate reader.
(7) In vitro assessment of cytolytic function of the engineered T-cells (engineered for delivery function). FRα-CAR⁺CD3 and CD4 T-cells (with NFAT-RE inducible delivery function) were co-cultured with target (FRα⁺Luc2-2A-E2Crimson+A2780cis) cells (2500 cells) in 200 μL of complete growth medium in a single well of a 96-well plate. After a 24-hour co-culture, the manufacturer's protocol was followed to measure the reporter activity, e.g., Luc2 activity in the A2780cis cells using One-Glo® assay. Briefly, the Luc2 substrate was diluted in the cell lysis buffer provided with the One-Glo® assay and added to co-culture in the 96-well plate for assessing Luc2 activity. Following a brief incubation period of 10 minutes, the bioluminescence was read on a microplate reader.
(8) Quantification of IFNβ delivery from CD4 T-cell-based effector cells. T-cells (CD3, CD4, CD8) engineered for IFNβ delivery was stimulated using microparticles functionalized with FRα antigen and anti-CD28 antibodies at 1:3 cell-to-particle ratio. Three days after stimulation, IFNβ activity in the cell culture supernatant was determined using IFN-α/B Reporter HEK 293 Cells using QUANTI-Blue™ assay kit following manufacturer's instructions. Recombinant human IFNβ standard was run in parallel to determine the equivalent IFNβ activity in the culture supernatant. Unmodified T-cells and T-cells engineered to express FRα-CAR (without NFAT-RE inducible delivery function) were used as control. All experiments were run in triplicate and the data is represented as mean±SD.
(9) Assessment of the anti-tumor function of CD4 T-cell-based IFNβ delivering effector cells. Growth inhibitory effect of the secreted IFNβ from CD4 and CD8 T-cell-based effector cells was assessed in ovarian cancer cell lines (OVACR3 and A2780cis) and HEK293T/17 cells. IFNβ enriched media was serially diluted in a 96-well plate and 5000 cells/well of each cell line was added and incubated in a cell culture incubator (37 degree C., 5% CO₂, 95% humidity) for 3 days. Growth inhibition was determined by CellTiter-Glo® assay following manufacturer's protocol. All experiments were run in triplicate and the data is represented as mean±SD.
(10) In vivo validation of delivery function of the engineered T-cells (engineered for delivery function with NFAT-RE delivery system). The in vivo validation of the CD4 T-cell-based effector was performed in mice at SRI International in accordance with the guidelines from the Institutional Animal Care and Use Committee (Approval #22001). Twenty four 6-8-week-old female NOD.Cg-Prkde^{scid} Il2rg^{tmlWjl}/SzJ (NSG) mice were purchased from The Jackson Laboratory. After quarantine, the NSG mice were anesthetized and 2×10⁶ FRα⁺ MSLN^{Neg}Luc2-2A-E2Crimson⁺ A2780cis cells (in 18 mice) or FRα^{neg}MSLN⁺Luc2-2A-E2Crimson⁺A2780cis cells (in 6 mice) in 100 μL 1×PBS were i.p. implanted. The tumor growth was monitored every 3-4 days for the next 10 days using i.p. injected 150 mg D-Luciferin per kilogram (kg) of mouse dissolved in 1×PBS. At 11 days after implantation, implanted with FRα⁺ MSLNN^{Neg}Luc2-2A-E2Crimson⁺ A2780cis cells were randomized into three groups (6 mice each). The two groups (1 group of FRα⁺ MSLN^{Neg}Luc2-2A-E2Crimson⁺A2780cis cells and FRα^{neg}MSLN⁺Luc2-2A-E2Crimson⁺ A2780cis cells) were then treated with 2×10⁶ primary CD4 T-cells engineered for delivery function (e.g., FRα-CAR with NFAT-RE inducible Nluc reporter) or the control CD4 T-cells (without FRα-CAR but with NFAT-RE inducible Nluc reporter) or the no CAR CD4 T-cells (with NFAT-RE inducible Nluc reporter) every day for 5 days. The bioluminescent reporter (Nluc) activity was determined by i.p. injection of the Nano-Glo® substrate (1:20 dilution of the substrate in 1×PBS, equivalent to 0.5 mg per kg of mouse) on all days including on day 0, after treatment. Imaging was performed in an IVIS Lumina X5 imaging system. The data was quantified by analysis of the ROI using Living Image software. The tumor luminescence is plotted as the mean±SEM of total flux (photons/s) against days after treatment.
(11) Challenge of in vivo tumor with primary CD4 T-cell-based IFN-β delivery system. The in vivo tumor challenge mouse study was performed at SRI International in accordance with the guidelines from the Institutional Animal Care and Use Committee (Approval #22001). Eighteen 6-8-week-old male NOD.Cg-Prkdc^{scid} 112rg^{tm1Wjl}/SzJ (NSG) mice were purchased from The Jackson Laboratory. The NSG mice were anesthetized and 2×10⁶ FRα⁺Luc2-2A-E2Crimson⁺A2780cis cells in 100 μL 1×PBS were i.p. implanted. After 12 days, the mice were randomized into three groups (n=6 each) and were treated with 5×10⁶ FRα-CAR⁺CD4 T-cells (without NFAT-RE inducible delivery function) or FRα-CAR⁺ T-cell-based IFN-β delivery system (with NFAT-RE inducible delivery function) every day for 6 days. The third group was treated with rhIFNβ at 0.25 μg per mice in 100 μL 1×PBS every day for 6 days. The tumor growth was monitored every 3-4 days using i.p. injected 150 mg D-Luciferin per kg of mouse dissolved in 1×PBS. The luminescence imaging was performed in an IVIS Lumina X5 imaging system. The data was quantified by analysis of the ROI using Living Image software. The tumor luminescence is plotted as the mean±SEM of total flux (photons/s) against days after tumor implantation.
(12) Statistical analysis. Results are expressed as an arithmetic mean±SD if not otherwise stated. For each run, each sample was measured in a technical replicate. Values of p<0.05 were considered to indicate statistical significance, as determined using ANOVA with Tukey's multiple comparison test, or as specified. Analyses were performed using Prism version 8.0 (GraphPad Software).

As previously described, various experimental embodiments were directed to optimizing the process of forming genetically engineered effector cells from primary T-cells. In such experimental embodiments, a range of process parameters were assessed for increasing the production yield of the primary T-cells engineered for delivery function. Compared to the common spinoculation-based method, the transduction yield was enhanced about 2.5-fold by restricting the transduction reaction volume for maximizing the lentivector-to-T-cell contact. Cell density and cytokines used in the expansion process were adjusted to achieve>100-fold expansion of the T-cell-based effector cell in 14 days, and the function of these cells was validated in vivo using intraperitoneally implanted tumor cells. The primary T-cell-based effector cell has human applications because it can be scaled and administrated to express a broad range of therapeutic proteins (e.g., cytokines, interferons, enzymes, agonists, and antagonists) at the disease site, obviating the need for systemic delivery of large doses of these proteins. In such experimental embodiments, primary T-cell have been transformed into a platform for synthesizing complex biologics directly at the disease site with precise timing and location. As noted above, unlike the current status-quo of first-order drug delivery systems that presents systemic biodistribution and can affect healthy tissues, this technology can be used to synthesize engineered proteins so as to exert therapeutic effects by autocrine or paracrine signaling only at the disease site without affecting healthy tissues. In vivo experiments confirmed the synthesis of functional proteins by the engineered cells.

As previously shown by FIG. 2A, various embodiments used a CAR with specificity for FRα and a reporter enzyme to modified T-cells and represent the desired biologic, using a process to efficiently scale the primary T-cell-based effector cell formation. The DNA template of the antigen-sensing scFv domain and the reporter enzyme can be exchanged to redirect the specificity of this platform to other clinically relevant antigen and express a protein for a sensing and/or therapeutic function(s).

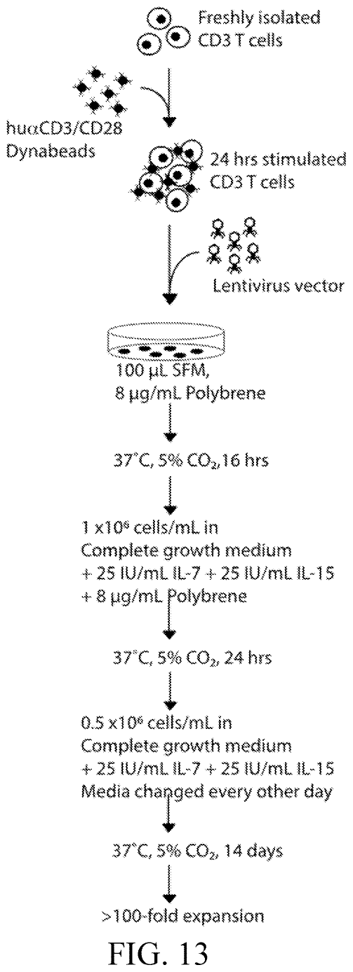

FIG. 13 illustrates an example process for forming genetically engineered effector cells from primary T-cells, in accordance with the present disclosure. As shown, isolated CD3 T-cells were activated with anti-CD3/CD28 Dynabeads™ (cell: particle of 1:3) for 24 hours, transduced the activated T-cells by increasing lentivector-to-T-cell contact in 0.1 mL volume for 16 hours, and expanded the transduced cells at 0.5×10⁶ cells/mL in complete growth medium supplemented with IL-7 and IL-15, with half-media changes every 2-3 days for 14 days. Various experiments were directed to assessing different process parameters and the effect on transducing primary T-cells with lentivectors.

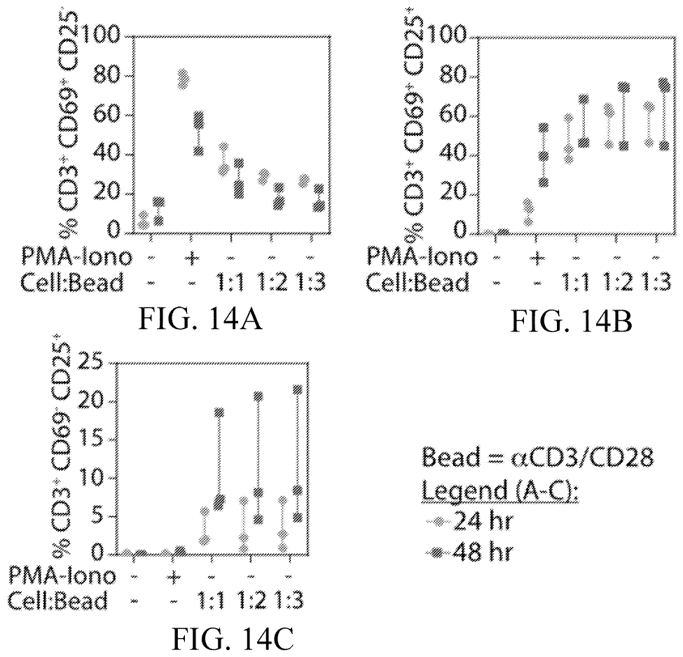

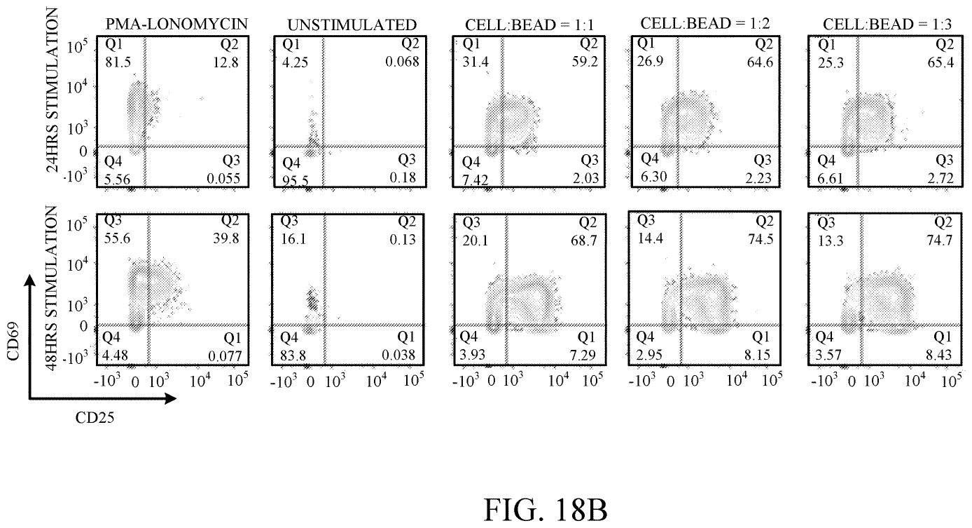

FIGS. 14A-15D illustrate example effects of various parameters on the lentivector transduction of primary T-cells, in accordance with the present disclosure. More specifically, FIGS. 14A-14C show the effect of the cell-to-particle ratio on early (CD69+CD25⁻) (FIG. 14A), peak (CD69+CD25+) (FIG. 14B), and late (CD69-CD25+) (FIG. 14C) activation of CD3 T-cells (see also FIGS. 18A-18B for the gating strategy using a representative fluorescence-activated cell sorting (FACS) plot). FIG. 14B shows that 60% of CD3 T-cells progress to peak activation (CD69+CD25+) within 24 hours after stimulation by particles at a cell-to-particle ratio of 1:3. As such, this ratio was used in the formation and/or manufacturing process in various experiments. FIG. 14D shows a comparison of the transduction efficiency of T-cells activated by particles at a ratio of 1:3 to chemically-activated T-cells (Phorbol 12-myristate 13-acetate and Ionomycin, PMA/Io). Although the transduction efficiency was similar in magnitude (42% verses 40%, n=3), the yield for producing engineered FRα-CAR⁺ T-cell was higher when activated with particles compared to PMA/Io (75% vs. 60%, n=3). The transduction efficiency was about 15% in non-activated T-cells.

As shown by FIG. 14A-14C, T-cell activation marker (CD25, CD69) expression in CD3 T-cells (n=3 donors) was assessed by flow cytometry at 24- or 48-hours after stimulation by chemicals (Phorbol 12-myristate 13-acetate (30 nM) and Ionomycin (1 μM), PMA/Io) or by different cell-to-particle (Dynabeads™ loaded with anti-human CD3 and anti-human CD28) ratios. Strategy for evaluating CD3 T-cell activation is presented in FIGS. 18A-18B. Different stages of T-cell activation are shown in early activation (CD69+CD25⁻) (FIG. 14A), peak activation (CD69⁺CD25⁺) (FIG. 14B), and late activation (CD69-CD25⁺) FIG. 14C).

As shown by FIG. 15A, FRα-CAR expression (% FRα-CAR⁺ T-cells on left Y-axis) and T-cell viability (% Viability on right Y-axis) was assessed by flow cytometry after transducing stimulated and unstimulated primary T-cells. As shown by FIGS. 15B-15D, FRα-CAR expression (% FRα-CAR⁺ T-cells on left Y-axis) and T-cell viability (% Viability on right Y-axis) was assessed by flow cytometry after varying factors affecting transduction including: (i) size of the genetic payload (chimeric antigen receptor (CAR) only, 5.6 kb vs T-cell-based delivery system comprising of CAR and NFAT-RE inducible transgene, 7.2 kb), see FIGS. 7A-7D for schematics (FIG. 15B), (ii) lentivector pseudotype (RD114 vs VSV-g) (FIG. 15C), and (iii) lentivector transduction methods (Method a: spinoculation at 800 gravitational force (G) in 0.5 mL for 1.5 hours and 14.5 hours in cell culture incubator; Method b: 1.0 mL reaction volume for 16 hours in cell culture incubator; Method c: 0.1 mL reaction volume for 16 hours in cell culture incubator) (FIG. 15D). Transduction efficiency was determined after 5 days. All results are represented as mean±SD. Statistical analysis and p values for FIG. 15A and FIG. 15D were determined by one-way ANOVA and Tukey's multiple comparison test. Statistical analysis and p values for FIG. 14C was determined by Student's t-test, two-tailed; * p<0.05, ** p<0.01, *** p<0.001 and **** p<0.0001. FIG. 15B was determined by Student's t-test, two-tailed. FIG. 15C, although analyzed by Student's t-test, no significance was obtained.

FIGS. 15B-15D show a comparison of other parameters that affect the transduction efficiency of primary T-cells. Experiments were directed to assessing improvement as the percentage of modified primary T-cells (% FRα-CAR⁺ T-cells, left y-axis) and the number of live primary T-cells (% viability, right y-axis) in the culture 5 days after transduction.

FIG. 15B shows the effect of the size (LTR-to-LTR) of genetic payload, 7.2 kb (for the T-cell-based delivery system comprising the CAR and NFAT-RE inducible transgene) versus 5.6 kb (CAR only), using the same plasmid vector (FIGS. 7A-7B). No difference in cell viability was observed, but the resulting transduction yield for transducing primary T-cells with the 7.2 kb genetic payload was about 50% less compared to that of the 5.6 kb genetic payload (62.85±14% versus 33.8±15%, n=4 donors). This is consistent with a report on the decreased efficiency of lentivector transduction with an increase in transgene length.

To address the reduced yield in generation of the primary T-cell-based effector cells, two pseudotyped lentivirus vectors for transducing T-cells were compared: (i) VSV-g envelope protein from vesicular stomatitis virus, and (ii) RD114 envelope protein from infectious feline endogenous retrovirus, as described by Zhang et al., Transduction of Bone-Marrow-Derived Mesenchymal Stem Cells by Using Lentivirus Vectors Pseudotyped with Modified RD114 Envelope Glycoproteins, Journal of Virology 78 (3): 1219-1229 (2004), which is incorporated herein in its entirety for its teaching. The VSV-g envelope protein is accepted for engineering T-cells, and RD114 has been reported to improve efficiency in engineering CD34 hematopoietic cells and CAR T-cells. The results are presented in FIG. 15C. No significant difference in transduction efficiencies (RD114:37.1±9.5%; VSV-g: 34±10.6%) or viability of the engineered primary T-cells was observed. Given the acceptance of VSV-g pseudotyped lentivectors, it was used in various experiments.

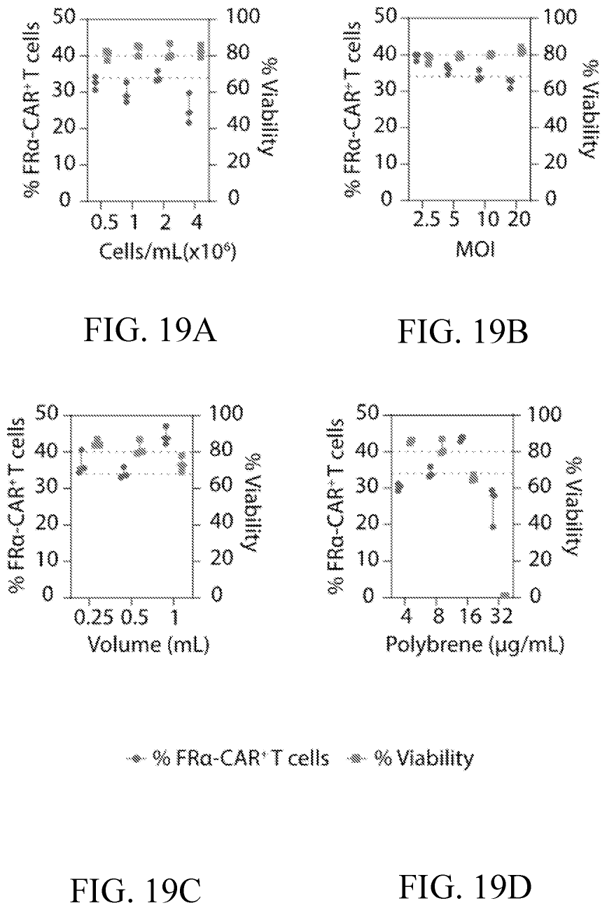

Experiments were further directed to assessing the effect of: (i) cell concentration (FIG. 19A), (ii) MOI (FIG. 19B), (iii) transduction reaction volume (FIG. 19C), and (iv) polybrene concentration (FIG. 19D) on transducing primary T-cells. Based on the tested range of each parameters studied, a spinoculation process was used where 1 million primary T-cells were transduced at an MOI of 10 in 500 μL with 8 μg/mL polybrene.

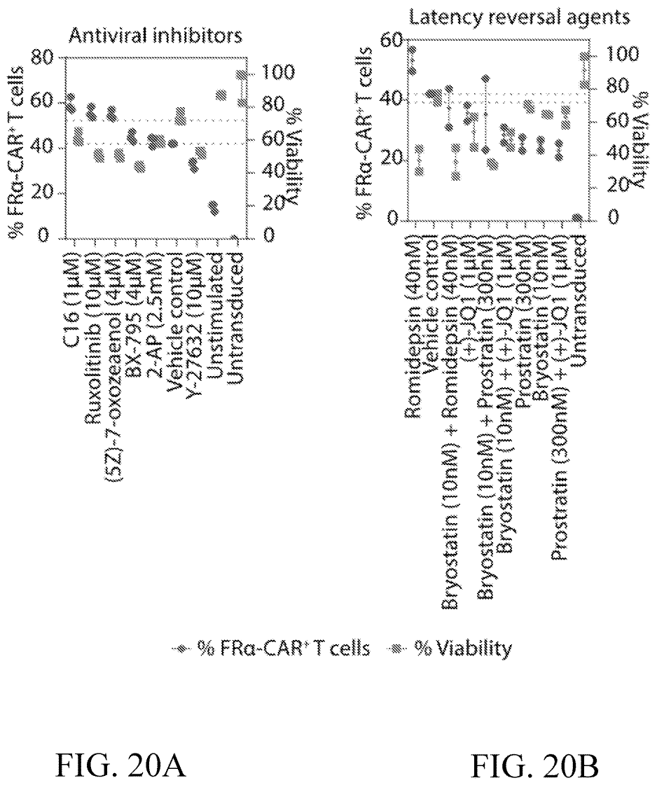

To further enhance the process yield, two different classes of chemical additives, AVIs and LRAs were assessed. The results are detailed in FIGS. 20A-20B. The intracellular antiviral response impedes the transduction efficiency of primary T-cells when lentivirus-based vectors are used. To address this issue, the use of AVIs was assessed to suppress the intracellular immunity against infection from the lentiviral vectors and potentially increase the transduction yield. Inhibition of intracellular antiviral signaling has improved T-cell transduction.

Several AVIs were assessed for inhibiting three different antiviral pathways, and the results are shown in FIG. 20A. These included: (i) the TANK-binding Kinase 1 (TBK1) pathway: BX795 and (5Z)-7-Oxozeaenol, (ii) the RNA-dependent protein kinase (PKR) pathway: 2-aminopurine (2-AP) and C16, and (iii) other pathways, such as STAT (ruxolitinib) and Rho (Y-27632) signaling. Concomitant treatment with AVIs for PKR or TBK1 pathways during T-cell transduction increased transduction of primary T-cells (FIG. 19A).

FIG. 20B shows the results with LRAs as additives in the T-cell transduction and expansion media. The LRAs facilitate unfolding of the chromatin structure that determines DNA accessibility of the host genome and retroviral gene integration. A preferential bias for the site of gene integration was strongly displayed by gamma-retroviruses, delta-retroviruses, and lentiviruses with DNA insertion into transcriptionally active chromatin. A subset of LRAs, such as protein kinase C (PKC) agonists and/or its combination with inhibitors of bromo extra terminal (BET) or histone deacetylases (HDAC), were assessed for their ability to improve T-cell transduction with large lentiviral constructs. The LRA romidepsin increased the percentage of the engineered T-cells (55%) versus vehicle control (42%) (FIG. 20B), but the percentage of live cells was only 40% compared to 75% in the control, rendering romidepsin unfit for use in combination with lentivectors.

The above results were used to create an integrated process flow, and then validate it by transducing primary T-cells from three human donors (e.g., n=3). For improved transduction, an approach of restricting the volume of transduction reaction was used. Three different methods were assessed to confine the volume of transduction reaction before adding IL-2 to each reaction mix: (Method a) spinoculating in 0.5 mL at 800G in a well of a 24-well plate for 1.5 hours followed by incubating the reaction in a cell culture incubator (37 degrees C., 5% CO₂, 95% humidity) for another 14.5 hours; (Method b) using a defined reaction volume of 1.0 mL in a well of a 6-well plate for 16 hours in cell culture incubator; and (Method c) restricting the volume within a 0.1 mL drop in a well of a 6-well plate for 16 hours in cell culture incubator. 1×10⁶ primary T-cells were transduced with the lentivector particles at an MOI of 10 in serum-free medium supplemented with 8 μg/mL polybrene and diluted the transduction reaction with complete growth medium supplemented with 50 U/mL IL-2 after 16 hours in all three methods. The process efficiency was assessed five days after the transduction (FIG. 15D). The results showed 32% transduction at 44% viability with spinoculation (Method a); 53% transduction at 8% viability when using 1.0 mL reaction volume (Method b); and 60% transduction at 60% viability when restricting the reaction volume within the 0.1 mL drop (Method c). Limiting the reaction volume to increase lentivector-to-cell contact allowed Method c to produce about 2.5-fold more engineered primary T-cells compared to the spinoculation method (Method a). Based on these results, Method c was selected, confining the transduction reaction within 0.1 mL drop, as part of the optimized production process.

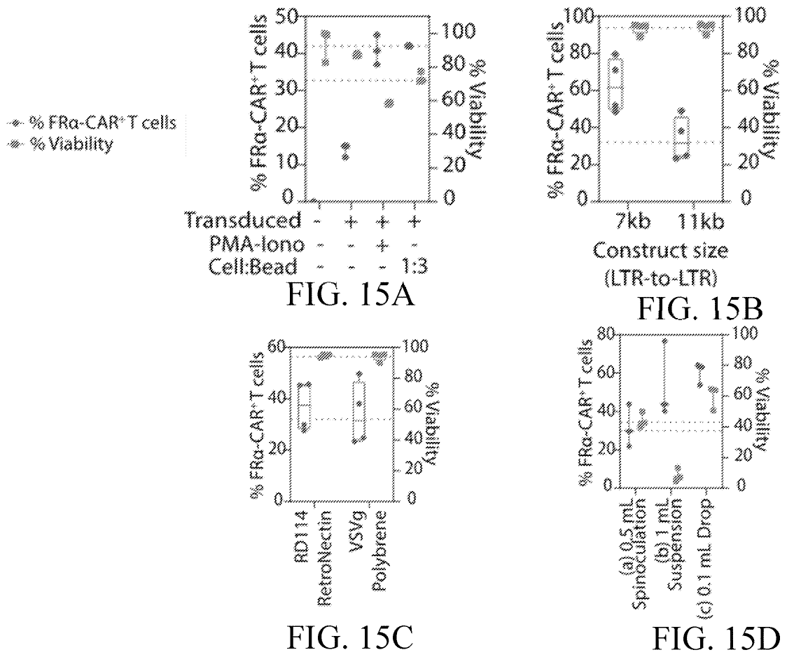

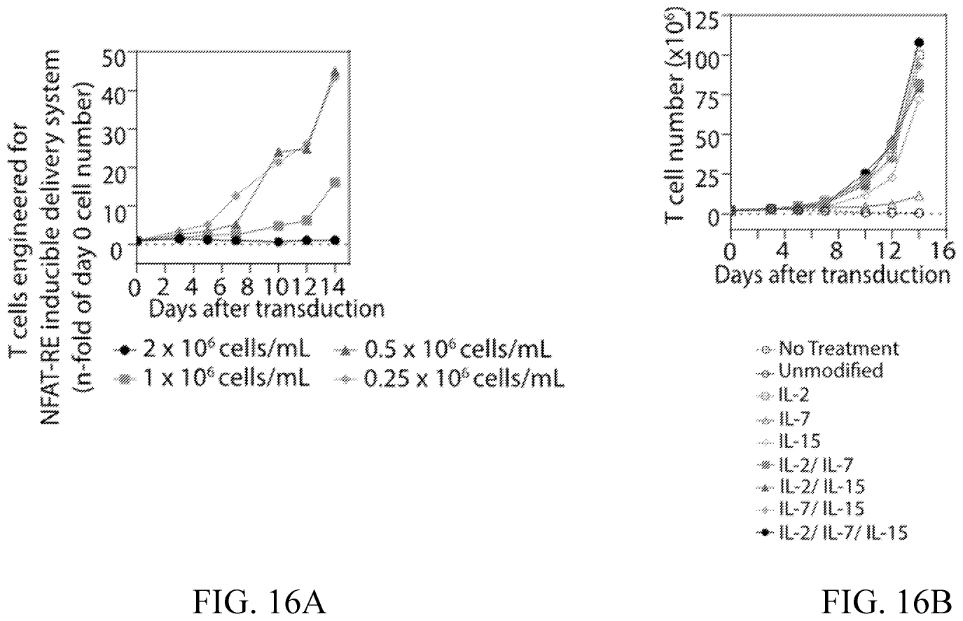

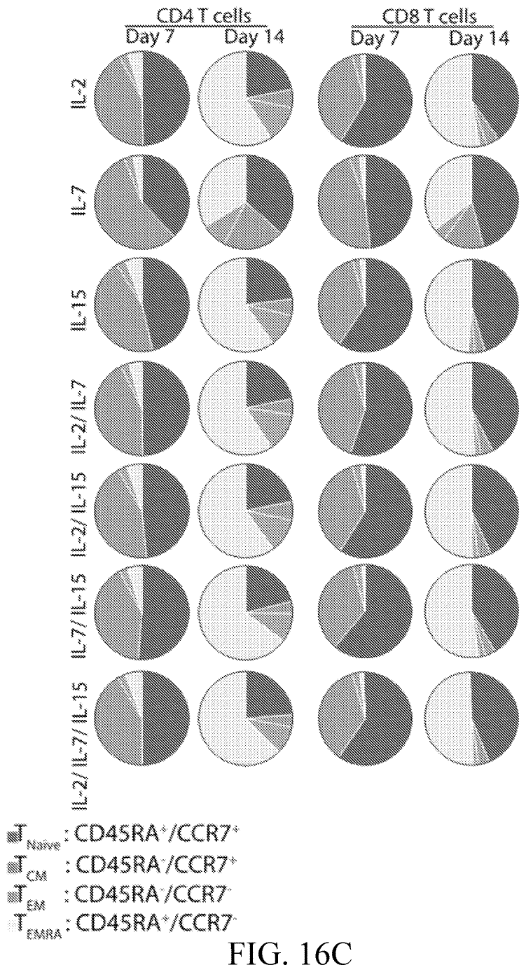

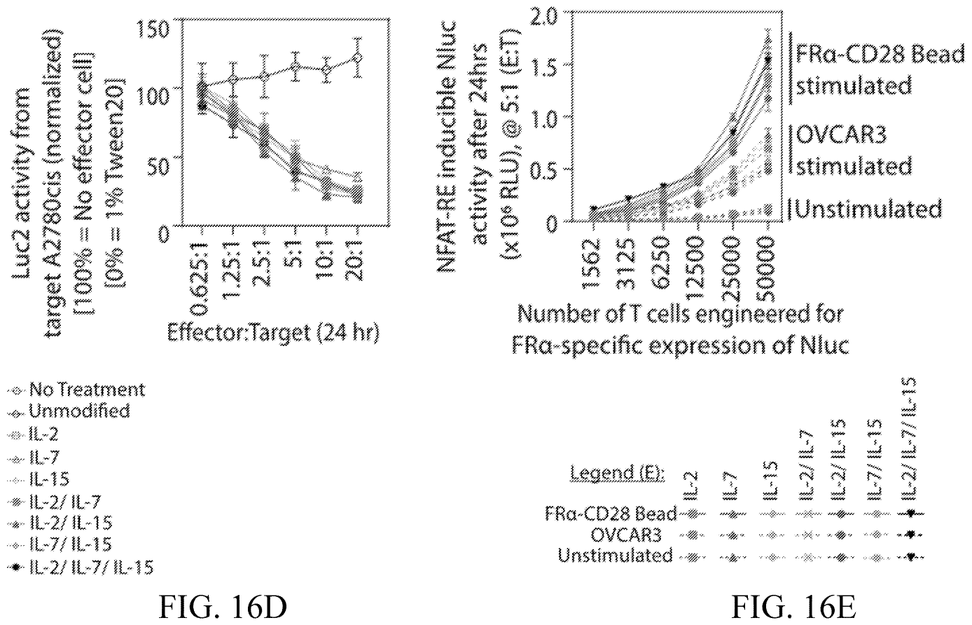

Various experiments were directed to assessing factors affecting the expansion of the engineered primary T-cell based effector cells. FIGS. 16A-16E illustrate example effects of various parameters on the expansion of primary T-cells, in accordance with the present disclosure. In particular, T-cell density and cystokins were assessed for the impact on in vitro expansion of engineered primary T-cells. Numerical expansion of the engineered primary T-cells was assessed at different cell densities (FIG. 16A) and when supplemented with different cytokines (IL-2, IL-7, IL-15, and combinations thereof) (FIG. 16B). FIG. 16C shows naïve/memory T-cell phenotype (naïve (T_{N}: CD45RA⁺/CCR7⁺), central memory (T_{CM}: CD45RA/CCR7⁺), effector memory (T_{EM}: CD45RA/CCR7⁻), and terminally differentiated effector memory (T_{EMRA}: CD45RA⁺/CCR7⁻) as was assessed by flow cytometry in CD4 and CD8 T-cells at day 7 and 14 of in vitro expansion using the same cytokine combinations. FIG. 16D shows results of FRα-specific engineered primary T-cells expanded in the same cytokine combinations induced cytolysis in FRα⁺Luc2-2A-E2Crimson⁺A2780cis target cells in a dose-dependent manner. FIG. 16E shows results of FRα-specific engineered primary T-cells expanded in the same cytokine combinations induced effector function, e.g., the delivery function, as represented by the NFAT-RE inducible NanoLuc® (Nluc) reporter activity in a dose-dependent manner. The FRα-specific engineered primary T-cells were stimulated at 5:1 effector-to-target ratio (E:T) by particles conjugated to the target FRα antigen and CD28 co-stimulation molecules or by OVCAR3 cells that endogenously express FRα. All results are represented as mean±SD. Statistical analysis and p values for FIG. 16E was determined by one-way ANOVA and Tukey's multiple comparison test, *p<0.05.

FIG. 16A shows the effect of cell density on numerically expanding these engineered primary T-cells in the presence of 50 IU/mL IL-2. In a 14-day culture, the engineered T-cells expanded around 45-fold at cell densities of 0.5×10⁶ cells/mL and 0.25×10⁶ cells/mL, compared to the around 16-fold expansion at 1×10⁶ cells/mL.

FIG. 16B shows the expansion results of engineered primary T-cells in the presence of different cytokines (IL-2, IL-7, IL-15, and combinations thereof). The engineered T-cells were maintained at 0.5×10⁶ cells/mL by adding complete media every other day and including the respective cytokine supplements (50 IU/mL IL-2, 25 IU/mL IL-7, 25 IU/mL IL-15). In a 14-day culture, a growth trend was observed that ranged from around 70-fold with IL-15 to around 110-fold when supplemented with combinations of IL-2, IL-7, and IL-15.

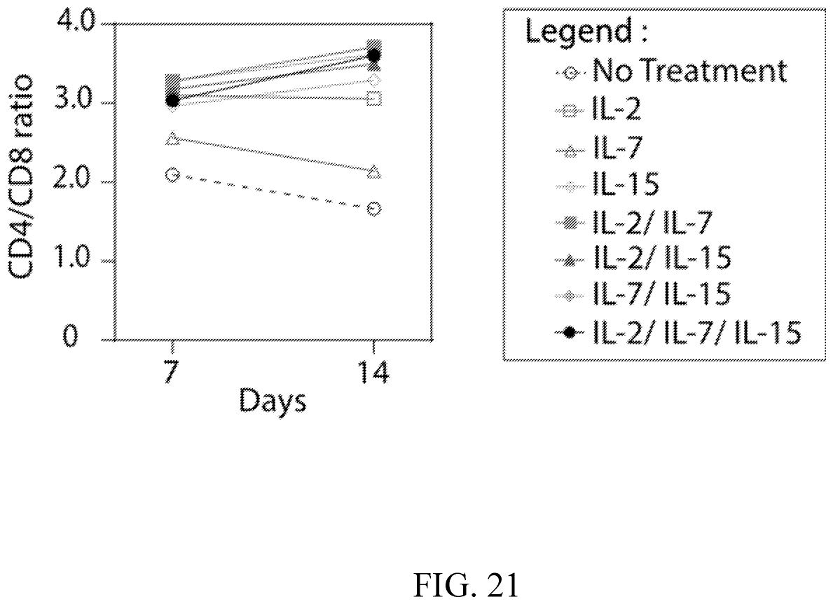

FIG. 16C shows phenotypic changes in the engineered primary T-cells when cultured in different cytokine cocktails, as detailed in FIG. 16B. The CD4 and CD8 subsets were analyzed at days 7 and 14 (see FIG. 21 for CD4/CD8 ratios) for naïve (T_{N}), central memory (T_{CM}), effector memory (T_{EM}), and terminally differentiated effector memory (T_{EMRA}) phenotypes using the markers CD45RA and CCR7. At day 7, >90% of both CD4 and CD8 T-cells were composed of T_{N} (CD45RA⁺/CCR7⁺ about 50%) and T_{CM} (CD45RA⁻/CCR7⁺ about 42%) compartments. After expansion from day 7 to day 14, as demonstrated in FIG. 16B, the T_{N} compartment of CD4 T-cells was reduced to less than 25%, and the T_{CM} compartment was reduced to about 6%. The expansion enriched the effector memory phenotypes characterized as T_{EM} (CD45RA/CCR7⁻) from about 3% on day 7 to about 13% on day 14 and T_{EMRA} (CD45RA⁺/CCR7) from about 5% on day 7 to about 60% on day 14.

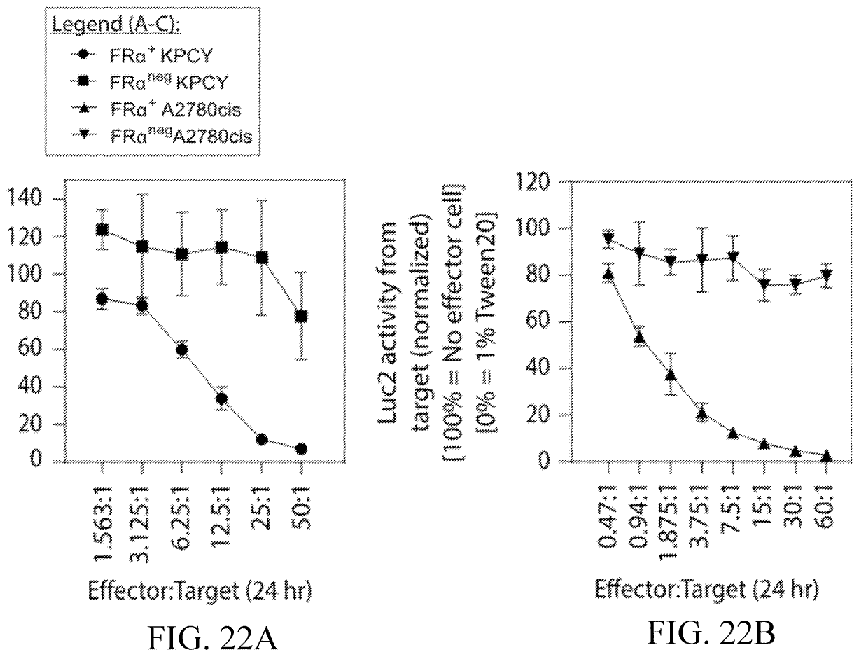

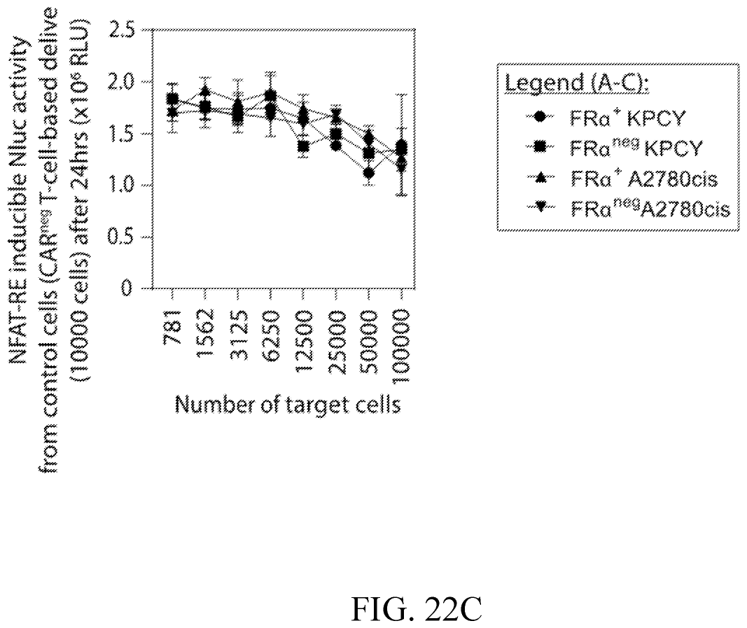

A similar observation was made in the CD8 T-cell subset. On day 7, the CD8 T-cell subset had a composition of about 58% T_{N}, about 37% T_{CM}, about 3% T_{EM}, and about 2% T_{EMRA}. On day 14, the composition was about 40% T_{N}, about 4% T_{CM}, about 3% T_{EM}, and about 53% T_{EMRA}. Except for the IL-7-supplemented culture, in which the T_{N} compartment of the engineered primary T-cells had negligible change from day 7 to day 14, all other cytokines induced T-cell expansion and had similar effects on the four phenotypes. This is in consensus with results showing the use of IL-7 for generating less differentiated CAR T-cells that have stem-like T-phenotypes. The T-cells showed notable enrichment of the T_{EMRA} compartment while the T_{CM} compartment was significantly reduced; this effect was least pronounced in the IL-7-supplemented culture. Although the IL-7-only culture showed reduced expansion at eleven-fold (FIG. 16B), it produced a higher fraction of naïve- and central-memory T-cells known for superior antitumor effect. Indeed, IL-7 treated T-cells with their high antigen-stimulated proliferation potential along with persistence and superior effectiveness have been used to reduce the number of CAR T-cells in a dose required to exert a clinical response. In addition to the proliferation, the cytolytic function of the primary T-cells engineered was observed into the delivery platform towards FRα-antigen expressing A2780cis tumor cell line (FIG. 16D) was proportional to the effector-to-target ratio (E:T) regardless of the cytokine composition used to expand different T-cell cultures. The target specific cytolytic function of the FRα-CAR cells was further supported by two independent tumor cell lines (A2780cis and KPCY) engineered for FRα-antigen expression compared to the respective antigen negative control (see FIG. 22A-22B).

The engineered effector function, e.g., the delivery function, as represented by the NFAT-RE inducible NanoLuc® (Nluc) reporter protein activity in the same T-cell cultures is shown in FIG. 16E. The engineered primary T-cells were expanded for 14 days and stimulated by beads (conjugated to the FRα antigen and anti-CD28 antibody, e.g., FRα-antigen/anti-CD28) or by OVCAR3 cells (expressing endogenous FRα antigen). After 24 hours of stimulation, a two-fold higher delivery function was observed in the engineered primary T-cells when FRα-antigen/anti-CD28 particles were used for stimulation compared to OVCAR3 cells, and had a six-fold increase in the delivery function compared to that observed from the unstimulated engineered primary T-cells.

Although the IL-7-expanded engineered primary T-cells showed peak delivery function, it was not significantly different from the engineered primary T-cells expanded with other cytokine combinations. Unlike the engineered primary T-cells expanded in IL-7 only, those expanded with the combination of IL-7 and IL-15 exhibited enhanced proliferation. It is believed that IL-7 and IL-15 support long-term persistence and memory responses of the T-cells, and such cytokines were selected as cytokine supplements for expanding the primary T-cells engineered for cell-based delivery of proteins.

To summarize, the above experiments encompassed optimizing multiple process parameters and assessing their effects on the in vitro performance of the T-cell-based delivery platform. In particular, superior results were obtained when thawed T-cells were activated with anti-CD3/CD28 Dynabeads™ (cell: particle of 1:3) for 24 hours, transducing the activated T-cells by increasing lentivector-to-T-cell contact in 0.1 mL volume for 16 hours, and expanding the transduced cells at 0.5×10⁶ cells/mL in complete growth medium supplemented with IL-7 and IL-15, with half-media changes every 2-3 days for 14 days. These optimized parameters were employed in the subsequent in vivo validation studies below, and are described in detail.

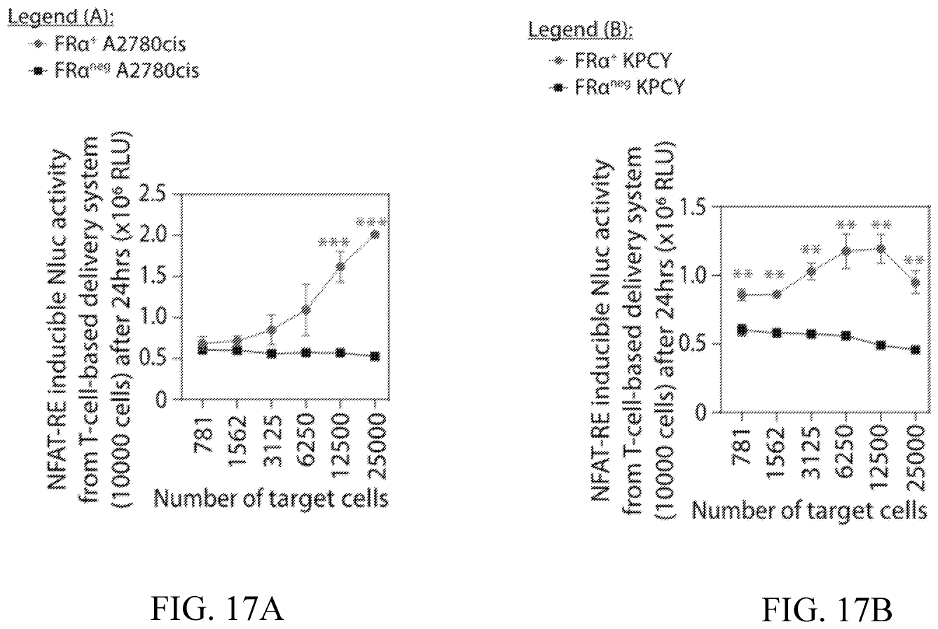

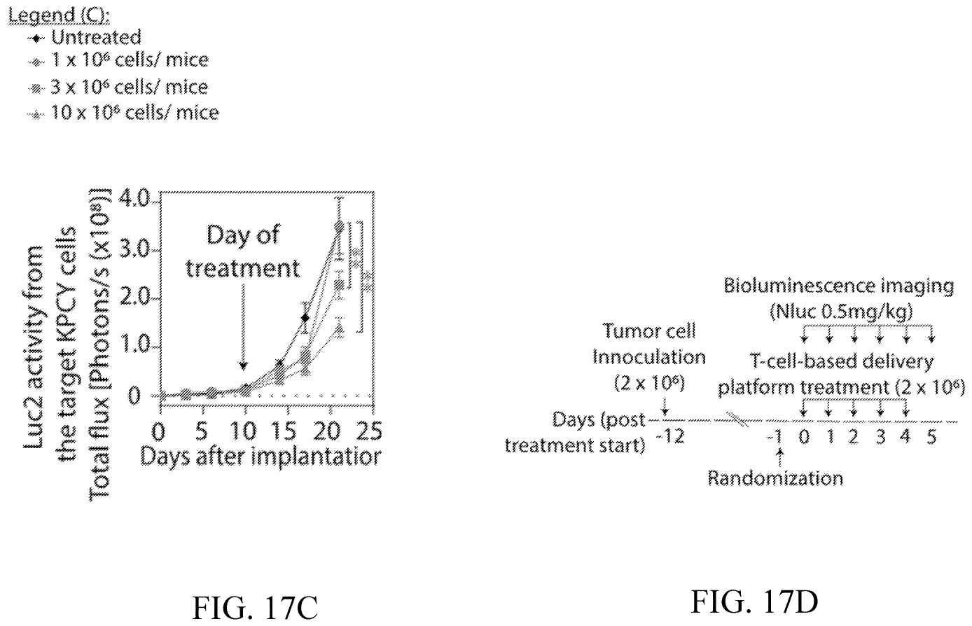

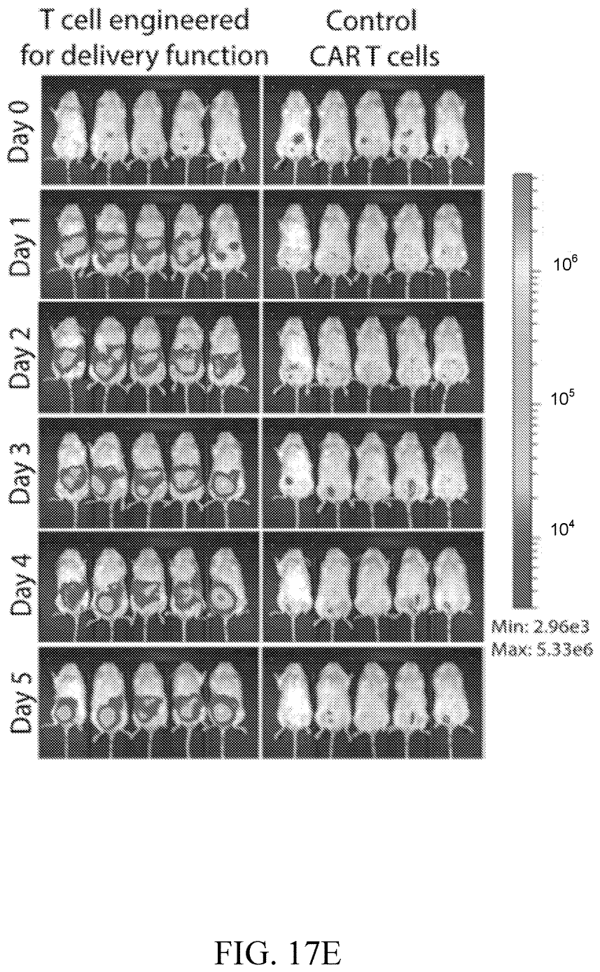

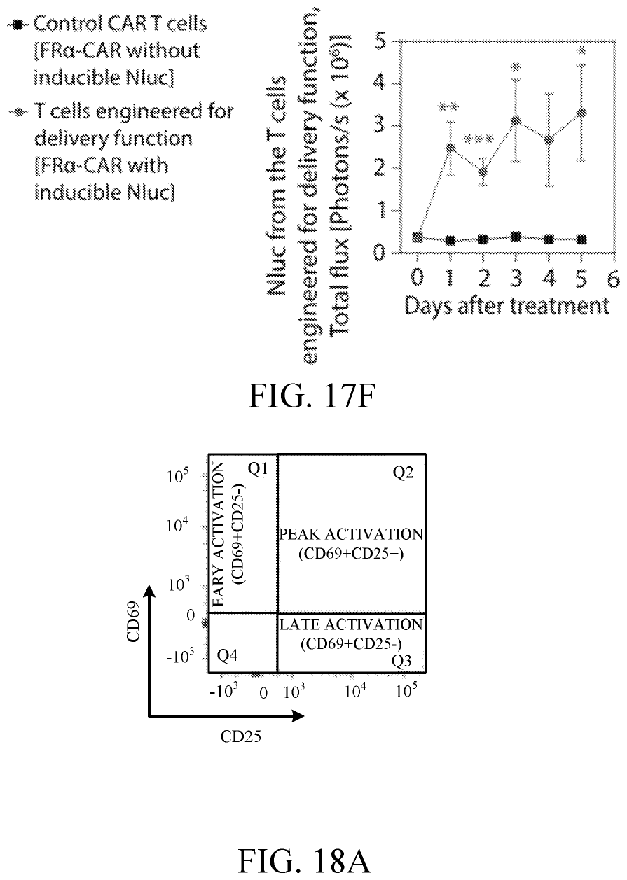

Various experiments were directed to functionally validating the engineered primary T-cell based delivery platform. FIGS. 17A-17F illustrate functional validation of the effector cells formed from a primary T-cell, in accordance with the present disclosure. For example, FIGS. 17A-17F show results of in vitro validation of target-specific delivery function proportionate to the disease burden. FRα-specific primary T-cells engineered for the NFAT-RE inducible delivery function showed proportionate increase in reporter activity when co-cultured with target, FRα⁺A2780cis (FIG. 17A) and FRα⁺KPCY cells (FIG. 17B), compared to their respective non-target (FRα^{neg}) control cells. FIG. 17C shows results of CAR T-cells formed using the process described above and developed for T-cell-based effector cells to reduced tumor burden. Tumor regression was observed in i.p. KPCY tumors in NSG mice when treated with FRα-specific CAR T-cells in a dose-dependent manner (n=5 mice per group). Bioluminescence (Luc2 activity) from the i.p. tumors was used to assess the tumor burden in vivo. Statistical analysis was performed using 2-way ANOVA and Tukey's multiple comparison test. There was a statistically significant interaction between days and FRα-specific CAR T-cell doses on tumor burden [F(18, 96)=4.595, p<0.0001]. FIGS. 17D-17F).

The primary T-cell-based effector cells formed using the same process was functional in vivo in an antigen-specific manner (n=5 mice per group). FRα-specific primary T-cells engineered for the NFAT-RE inducible delivery function were i.p. injected in i.p. FRα⁺A2780cis tumor-bearing NSG mice at 24-hour interval for 5 days and NFAT-RE inducible effector (Nluc) activity was measured for 6 days including the day of injection as a baseline to assess the delivery function. A control group was included to assess any background signal that may arise from using the Nluc substrate with Luc2⁺tumor cells and injected with FRα-specific primary CAR T-cells (engineered without the NFAT-RE inducible effector (Nluc)) to maintain equivalent tumor burden. More specifically, FIG. 17D shows schematic of dosing, treatment, and imaging schedules, FIG. 17E shows representative bioluminescent images, and FIG. 17F shows quantification. All results are represented as mean±SEM. Statistical analysis and p values for FIG. 17A, FIG. 17B, and FIG. 17F were determined by multiple t-test using Holm-Sidak method; * p<0.05, ** p<0.01, and *** p<0.001.

In more detail, the target-specific, delivery function proportionate to the disease burden was assessed in vitro by co-culturing the FRα-specific primary T-cells engineered for the NFAT-RE inducible delivery function against target cells, A2780cis (FIG. 17A) and KPCY cells (FIG. 17B). Compared to the non-target cells, e.g., antigen-negative cells (FRα^{neg} A2780cis, FRα^{neg}KPCY), co-culture with antigen-positive target cells showed a proportionate and significant increase in delivery function, Nluc reporter activity, with increase in target cell number. However, the control cells (e.g., primary T-cells engineered for the NFAT-RE inducible delivery function but without CAR) did not exhibit the delivery function when co-cultured with the same target and non-target cells (FIG. 22C). In further experiments, it was verified that the aforementioned process does not compromise the inherent cytolytic function of CAR T-cells.

KPCY2838c3 pancreatic ductal adenocarcinoma cells derived from KPCY mice were engineered to express human FRα antigen and Luc2 (FRα⁺Luc2⁺KPCY cells) for assessing tumor growth, and 0.5×10⁶ were i.p. implanted in NSG mice. FRα-CAR⁺T-cells (without the NFAT-RE inducible Nluc reporter) were expanded for 16 days and injected i.p. to challenge 10-day old FRα⁺Luc2⁺KPCY tumors. The results in FIG. 17C show a dose-escalation effect of the FRα-CAR⁺ T-cells (1×106, 3×10⁶, and 10×10⁶ FRα-CAR⁺ T-cells) on tumor regression. Compared to the group with no treatment, about 60% tumor regression was observed on day 21 when treated with 10×10⁶ FRα-CAR⁺ T-cells (p<0.01) and about 35% by 3×10⁶ FRα-CAR⁺ T-cells (p<0.01).

In various experiments, FRα-CAR⁺ T-cells were formed with the delivery function, e.g., upon engaging the target FRα antigen, the FRα-CAR activates the NFAT-RE signaling pathway to induce the expression of desired protein. The experiment schedule is detailed in FIG. 17D and the results are shown in FIGS. 17E-17F. More particularly, FIGS. 17E-17F show results of 2×10⁶ FRα⁺Luc2⁺A2780cis cells that were i.p. implanted in NSG mice. The 12-day-old xenograft tumors were i.p. treated with 2×10⁶ FRα-CAR⁺ T-cells (with NFAT-RE inducible Nluc reporter) on days 0, 1, 2, 3, and 4. A control group was included to assess any background signal from using the Nluc substrate on Luc2⁺tumor cells. This group was treated with i.p. injections of FRα-CAR⁺ T-cells without NFAT-RE inducible Nluc reporter (control FRα-CAR⁺ T-cells) to maintain an equivalent tumor burden. The effector (Nluc) activity was measured (FIG. 17E) and quantified (FIG. 17F) at baseline (day 0) as well as on days 1, 2, 3, 4, and 5. A significant increase in engineered effector activity (e.g., delivery function) was observed in the group treated with the FRα-CAR⁺ T-cells with the delivery function (e.g., with NFAT-RE inducible Nluc reporter), confirming the target-inducible in situ delivery function in the engineered primary T-cells.

FIGS. 18A-18B illustrate an example strategy for evaluating CD3 T-cell activation, in accordance with the present disclosure. FIG. 17A is a schematic of the gating strategy used for assessing early (CD69⁺CD25⁻), peak (CD69⁺CD25⁻), and late (CD69-CD25⁺) activated CD3 T-cells by flow cytometry. FIG. 18B are representative plots showing CD69 and CD25 expression in stimulated verses non-stimulated CD3 T-cells.

FIGS. 19A-19D illustrate example effects of additional factors on transduction of primary T-cells with lentivectors, in accordance with the present disclosure. FRα-CAR expression (% FRα-CAR⁺ T-cells on left Y-axis) and T-cell viability (% Viability on right Y-axis) were assessed by flow cytometry after varying factors affecting transduction. FIG. 19A shows T-cell concentration in a transduction reaction results, FIG. 19B shows lentivector MOI results, FIG. 19C shows transduction reaction volume results, and FIG. 19D shows polybrene concentration results. Transduction efficiency was determined after 5 days. All results are represented as mean±SD.

FIGS. 20A-20B illustrate an example exploratory screen of chemical additives for improving transduction of primary T-cells with lentivectors, in accordance with the present disclosure. FRα-CAR expression (% FRα-CAR⁺ T-cells on left Y-axis) and T-cell viability (% Viability on right Y-axis) was assessed by flow cytometry after concomitant treatment with AVIs (FIG. 20A), and LRAs (FIG. 20B) during lentivector transduction. Transduction efficiency was determined after 5 days. All results are represented as mean±SD.

FIG. 21 illustrates an example change in the proportion of CD3 T-cell subsets in response to cytokines, in accordance with the present disclosure. The CD4/CD8 ratio was assessed by flow cytometry in CD3 T-cells at day 7 and 14 of in vitro expansion when growth media was supplemented with different cytokines (IL-2, IL-7, IL-15, and combinations thereof).

FIGS. 22A-22C illustrate example antigen-specific cytolysis and NFAT-RE inducible delivery function, in accordance with the present disclosure. FRα-specific CAR T-cells formed using the above-described process induced cytolysis in FRα⁺Luc2-2A-E2Crimson⁺ KPCY(FIG. 22A) and FRα⁺Luc2-2A-E2Crimson⁺A2780cis (FIG. 22B) target cells in a dose-dependent manner compared to their respective antigen negative target cells. FIG. 22C shows Nluc activity from primary T-cells engineered for NFAT-RE inducible delivery function when co-cultured with antigen-positive and antigen-negative target cells for 24 hours.

II. Methods for the assessment of process parameters for forming the genetically engineered effector cells from primary T-cells.
(1) Materials and reagents. Table 2 lists sources for all materials, supplies, services, and equipment used in the above-described experiments.

TABLE 2
Resources Table

| | | |
|---|---|---|
| | | IDENTIFIER |
| REAGENT OR RESOURCE | SOURCE | (CAT#) |

Biological samples

| | | |
|---|---|---|
| Human primary CD3 T-cells | Stanford Blood | A1020 |
| | Center | |
| A2780cis | Sigma-Aldrich | 93112517 |
| OVCAR3 | ATCC | HTB-161 |
| KPCY2838c3 | Kerafast | EUP013-FP |
| HEK293T/17 | ATCC | CRL-11268 |
| NOD.Cg-Prkdc^{scid} Il2rg^{tm1Wj1}/SzJ (NSG) | Jackson Laboratory | 005557 |

Chemicals, peptides, recombinant proteins

| | | |
|---|---|---|
| Fetal bovine serum (FBS) | Sigma-Aldrich | F2442-500 ML |
| Dimethyl sulfoxide, DMSO | Sigma-Aldrich | D2650-100 mL) |
| GlutaMAX ™ | Gibco | 35050-061 |
| Penicillin streptomycin solution | Corning | 30-002-Cl |
| Human IL-2 | Miltenyi Biotec | 130-097-748 |
| Human IL-15 | PeproTech | 200-15-50 UG |
| Human IL-7 | PeproTech | 200-07-50 UG |
| Dynabead Biotin Binder | Invitrogen | 11047 |
| Phorbol 12-myristate 13-acetate (PMA) | Sigma-Aldrich | P1585 |
| Ionomycin (Io) | Sigma-Aldrich | I0634 |
| DMEM media | Corning | 10-013-CV |
| Transporter 5 ™ | Polysciences, Inc. | 26008-5 |
| RPMI1640 | Corning | 10-040-CV |
| Phosphate buffered saline (PBS) | Corning | 21-040-CV |
| without Ca⁺² and Mg⁺² | | |
| Puromycin dihydrochloride | ThermoFisher | A1113803 |
| | Scientific | |
| 2-aminopurine (2-AP) | Combi-Blocks | OR-1074 |
| C16 | EMD Millipore | 527451-5 MG |
| BX-795 | Cayman Chemical | 14932 |
| (5z)-7-oxozeaenol | Cayman Chemical | 17459 |
| Y-27632 | Tocris | 1254 |
| Ruxolitinib | Cayman Chemical | 11609 |
| Romidepsin | Cayman Chemical | 17130 |
| Bryostatin | EMD Millipore | 203811-10 UG |
| Prostratin | Cayman Chemical | 10272 |
| (+)-JQ1 | Cayman Chemical | 11187 |
| Biotinylated human Folate receptor 1 | Acro Biosystems | FO1-H82E2 |
| (FOLR1 -His Tag -Avi Tag) | | |
| D-Luciferin | Perkin Elmer | 122799 |
| Acridine Orange Propidium Iodide | Nexcelom | CS2-0106-5 mL |
| (AOPI) staining solution | | |
| Cell-staining buffer | Biolegend | 420201) |
| RetroNectin ® | Takara Bio | T100B |

Antibodies

| | | |
|---|---|---|
| Biotin anti-human CD3 | Biolegend | 317320 |
| Biotin anti-human CD28 | Biolegend | 302904 |
| PerCR/Cy5.5 anti-human CD3 antibody | Biolegend | 300328 |
| LIVE/DEAD ™ Fixable Aqua Dead Cell | Life Technologies | L34966 |
| Stain Kit | | |
| APC-streptavidin | Biolegend | 405207 |
| Brilliant violet 510 anti-human CD25 | Biolegend | 302639 |
| Alexa Fluor 700 anti-human CD69 | Biolegend | 310321 |
| Human naive/memory T-cell ID panel kit | Biolegend | 362201 |

Plasmids

| | | |
|---|---|---|
| Lentivector transfer plasmid | System Biosciences | CD510B-1 |
| psPAX2 | Addgene | 12260 |
| pMD2.G | Addgene | 12259 |
| pAdVAntage | Promega | E1711 |

Commercial assays

| | | |
|---|---|---|
| Nano-Glo ® assay | Promega | N1120 |
| One-Glo ® assay | Promega | E6110 |

Other materials

| | | |
|---|---|---|
| T150 Flasks | Corning | 430825 |
| Hera 150i CO2 incubator | Thermo Scientific | 50116047 |
| Cellaca slides | Nexcelom | CHT4-PD100- |
| | | 003 |
| 6-well incubation plate | Fisher scientific | FB012927 |
| 96-well plate | Greiner bio-one | 655083 |
| 0.45 μm filter | Corning | 430514 |
| 500 mL collection bottle | Corning | 430282 |
| Konica ultracentrifugation tube | Beckman Coulter | C14291 |
| Nexcelom K2 cellometer | Nexcelom | |
| DynaMag ™-2 sample rack | Life Technologies | 12322D |
| BD FACS Symphony A3 cell analyzer | BD Biosciences | |
| EnVision ™ Multilabel Plate Reader | Perkin Elmer | Model 2104- |
| | | 0010A |
| AMI HTX Spectral instrument | Spectral Instruments | |
| | Imaging | |
| IVIS Lumina X5 imaging system | Perkin Elmer | CLS148590 |
| Optima XPN-90 ultracentrifuge | Beckman Coulter | |

Software

| | | |
|---|---|---|
| FlowJo ® software | FlowJo LLC | |
| Aura Image software | Spectral Instruments | |
| | Imaging | |
| Living Image software | Perkin Elmer | 128110 |
| SnapGene software | GSL Biotech LLC | |
| Prism version 8.0 | GraphPad Software | |

(2) Preparations. Transfer plasmids with different genetic payloads were designed in SnapGene software and sub-cloned into the lentivector plasmid. Epoch Life Science, Inc. (Missouri City, TX) provided plasmid preparation services (chemical synthesis of DNA insert sequences, sub-cloning into respective vector backbones, and the amplification). Target cells [FRα⁺A2780cis (Sex: female), FRα⁺OVCAR3 (Sex: female)] engineered to express modified firefly luciferase (Luc2), as described in Repellin et al., Modular Antigen-Specific T-cell Biofactories for Calibrated In Vivo Synthesis of Engineered Proteins, Advanced Biosystems 2 (12): 1800210 (2018), and Repellin et al., Engineered Ovarian Cancer Cell Lines for Validation of CAR T Cell Function, Advanced Biosystems 4 (1): 1900224 (2020), were maintained in RPMI media [RPMI1640, 10% heat-inactivated FBS, and 1× penicillin streptomycin solution], each of which are incorporated herein by reference in their entireties for their teachings. The mouse pancreatic tumor cell line [KPCY2838c3 (Sex: female)] was maintained in DMEM media [DMEM, 10% heat-inactivated FBS, 1× GlutaMAX™, and 1× penicillin streptomycin solution. Phosphate buffered saline (PBS) without Ca⁺² and Mg⁺² was used to minimize cell clumping. When applicable, puromycin N-acetyltransferase was used as a selection marker and puromycin dihydrochloride (Puromycin) was used for selecting stable cell lines. A chemical activation of T-cells was achieved by treatment with 1 μM phorbol 12-myristate 13-acetate and 30 nM ionomycin (PMA/Io). Biotinylated human FRα protein was used to analyze FRα CAR expression on engineered primary T-cells.
(3) Method for producing lentivector particles. Lentivector particles were produced as described in Radhakrishnan et al., Lentivirus Manufacturing Process for Primary T-Cell Biofactory Production, Advanced Biosystems 4 (6): 1900288 (2020), which is incorporated herein by reference in its entirety for its teaching. Lentivirus manufacturing and its use in engineering cells were performed at SRI International following the guidelines of the approved Biological Use Authorization (BUA 17-05). Briefly, lentivector particles were prepared by packaging the corresponding transfer plasmid using 2^{nd}-generation lentivector system. HEK293T/17 (Sex: female) producer cells (12×10⁶) were seeded into tissue culture treated T150 flasks in 21 mL complete DMEM supplemented with 10% heat-inactivated FBS and 1× penicillin streptomycin solution and placed in a cell culture incubator (37 degrees C., 5% CO₂, 95% humidity). After 24 hours, transfer plasmid was co-transfected with 2^{nd} generation packaging plasmids (psPAX2, pMD2.G), and pAdV Antage plasmid at 4:3:1:0.4 wt-ratio, respectively (transfer plasmid: 12 μg, pxPAX2:9 μg, pMD2.G: 3 μg, pAdV: 1.2 μg). Transporter 5TM transfection reagent was used following the manufacturer's protocol (100 μL). Cell culture supernatant enriched with pseudo-viral particle was collected and replenished every 24 hours for 3 days (30 mL). The lentivector-enriched cell culture supernatant was clarified using a 0.45 μm filter. The supernatant was clarified by transferring it to a polypropylene Konica ultracentrifugation tube and centrifuging at 20,700 G in an SW32-Ti rotor using a Beckman Coulter Optima XPN-90 ultracentrifuge at 4 degrees C. for 2 hours. The resulting pellet was resuspended in 400 μL serum-free RPMI and aliquoted. It was expected to achieve an MOI of 10 when the lentivector particles produced in this process are used to transduce 1×10⁶ cells. The lentivector aliquots were stored at −80 degrees C. until use.
(4) Method for engineering primary T-cells. The primary T-cells engineered with NFAT-RE inducible drug delivery system and used in the in vivo validation studies (FIGS. 16A-16F) were formed using the procedures resulting from the cumulative developments reported herein. Human primary CD3 T-cells were purchased from the Stanford Blood Center (Palo Alto, CA). The T-cells were immediately counted and used fresh or were cryostocked using freezing media [90% heat-inactivated fetal bovine serum (FBS) and 10% dimethyl sulfoxide (DMSO)] in liquid nitrogen for future use. Biotin anti-human CD3 and Biotin anti-human CD28 antibody were loaded on Dynabead™ Biotin Binder paramagnetic particles following the manufacturer's instructions (anti-CD3/CD28 Dynabeads). Human primary T-cells were thawed (Day 0), resuspended in complete growth medium, and activated by anti-CD3/CD28 Dynabeads™ (cell: particle of 1:3). After 24 hours (Day 1), 1×10⁶ activated primary T-cells were transduced with the appropriate lentivector particles resuspended in 0.1 mL volume of serum-free RPMI at an MOI of about 10 and in the presence of 8 μg/ml polybrene. The 0.1 mL aliquots of transduction reaction mix were placed as drops in a tissue-culture treated 6-well plate and placed in a cell culture incubator (37 degrees C., 5% CO₂, 95% humidity) for 16 hours. After 16 hours of incubation (around Day 2), the transduced primary T-cells were cultured at 1×10⁶ cells/mL in complete growth medium supplemented with recombinant human IL-7 (25 IU/mL), recombinant human IL-15 (25 IU/mL), and 8 μg/mL polybrene. The cells were counted after another 24 hours (Day 3) and every other day thereafter using acridine orange and propidium iodide (AOPI) staining in a Nexcelom K2 cellometer. They were maintained at a concentration of 0.5×10⁶ cells/mL in complete growth medium supplemented with recombinant human IL-7 (25 IU/mL) and recombinant human IL-15 (25 IU/mL) and the media was replaced every 2-3 days with half media changes. No polybrene was added on Day 3 and beyond.
(5) Approach for improving the production of primary T-cell-based delivery system. The above process was deviated from when exploring the factors to improve the production of the cell-based delivery system. For transduction optimization, the following were varied: (i) the duration of activation (FIGS. 13A-13C), (ii) cell-to-particle ratio (FIGS. 14A-14C and FIG. 15A), (iii) construct size (FIG. 15B), pseudotyped lentivectors (FIG. 15C), and (iv) transduction methods (Method a, Method b, Method c) (FIG. 15D). For transduction optimization, the following were varied: (i) concentration of activated primary T-cells (cell count was varied in volume of 0.5 mL) (FIG. 18A), (ii) MOI (cell count of 1×10⁶, volume of 0.5 mL) (FIG. 19B), (iii) transduction reaction volumes (cell count of 1×10⁶, MOI of 10) (FIG. 19C), and (iv) polybrene concentrations (cell count of 1×10⁶, MOI of 10, volume of 0.5 mL) (FIG. 19D). To further explore the effect of AVIs [PKR inhibitor 2-aminopurine (2-AP) and C16, TANK-binding kinase inhibitors BX-795 and (5z)-7-oxozeaenol, rock inhibitor Y-27632 and STAT inhibitor ruxolitinib] (FIG. 20A) and LRAs [Romidepsin, Bryostatin, Prostratin and (+)-JQ1] (FIG. 20B), transduction buffer composition was varied by adding these chemicals. For optimizing the expansion of transduced T-cells, the starting cell concentration of the transduced cells (FIG. 16A), and growth cytokines (FIG. 16B-16E) were varied.
(6) Flow cytometry analysis. The production yield of engineered primary T-cells was determined by assessing the expression of FRα-CAR on the T-cells engineered for drug delivery (% FRα-CAR⁺ T-cells) and cell viability. Five days after transduction, about 1×10⁶ T-cells were collected and de-beaded by keeping the T-cell suspension on a DynaMag™-2 sample rack for 2 minutes to remove the Dynabead™ biotin binder particles. The de-beaded T-cells were washed in cell-staining buffer and stained for 1 hour at 4 degrees C. using an antibody cocktail containing biotinylated human FRα protein (FOLR1-His Tag-Avi Tag), PerCR/Cy5.5 anti-human CD3 antibody and the LIVE/DEAD™ Fixable Aqua Dead Cell Stain Kit. The cells were washed, and a secondary staining was performed for 1 hour at 4 degrees C. using APC-streptavidin. The samples were washed, resuspended in 200 μL Cell Staining Buffer, and analyzed with a BD FACS Symphony A3 (BD Biosciences). The data were further processed using FlowJo® software. To determine T-cell activation (FIG. 14A-14C), a single-staining step protocol was followed whereby the de-beaded T-cells were washed and stained for 1 hour at 4 degrees C. using an antibody cocktail containing PerCR/Cy5.5 anti-human CD3 antibody, brilliant violet 510 anti-human CD25 antibody, and Alexa Fluor 700 anti-human CD69 antibody. Gating was performed using unstained control sample upon fluorescence compensation. To assess the naïve/memory phenotype of expanded T-cells (FIG. 15C), the human naïve/memory T-cell ID panel kit was used. All samples were analyzed using BD FACS Symphony A3 (BD Biosciences) and the data were further processed using FlowJo® software.
(7) In vitro assessment of cytolytic function of the engineered T-cells (engineered for delivery function). FRα-CAR⁺ T-cell (with NFAT-RE inducible delivery function) were co-cultured with target (FRα⁺Luc2-2A-E2Crimson⁺ A2780cis) cells (2500 cells) in 200 μL of complete growth medium in a single well of a 96-well plate. After a 24-hour co-culture, the manufacturer's protocol was followed to measure the reporter activity, e.g., Luc2 activity in the A2780cis cells using One-Glo® assay. Briefly, the Luc2 substrate was diluted in the cell lysis buffer provided with the One-Glo® assay and added to co-culture in the 96-well plate for assessing Luc2 activity. Following a brief incubation period of 10 minutes, the bioluminescence was read on a microplate reader.
(8) In vitro assessment of delivery function of the engineered-cells (engineered for delivery function). FRα-CAR⁺ T-cell (with NFAT-RE inducible delivery function) were co-cultured with the targets (OVCAR3 or FRα-antigen/anti-CD28 Dynabeads™) at an effector-to-target ratio (E:T) of 5:1, in 200 μL of complete growth medium in a single well of a 96-well plate. After a 24-hours co-culture, the manufacturer's protocol was followed to measure the reporter activity, e.g., Nluc activity in the engineered primary T-cells using Nano-Glo® assay. Briefly, the Nluc substrate was diluted in the cell lysis buffer provided with the Nano-Glo® assay and added to the co-culture in 96-well plates to assess the Nluc activity. Following a brief incubation period of 3 minutes, the bioluminescence was read on a microplate reader.
(9) Challenge of in vivo tumor with CAR T-cells (control cells with the NFAT-RE inducible delivery system). The in vivo tumor challenge mouse study (FIG. 17A) was performed at the Molecular Medicine Research Institute (MMRI) in accordance with the guidelines from the Institutional Animal Care and Use Committee (Approval #22-001). Twenty 6-8-week-old male NOD.Cg-Prkdc^{scid} 112rg^{tmlWjl}/SzJ (NSG) mice were purchased from The Jackson Laboratory. The KPCY2838c3 cells, engineered to express human FRα, Luc2, and E2Crimson using lentiviral vector (FRα⁺Luc2-2A-E2Crimson⁺KPCY2838c3 cells), were selectively expanded in the presence of puromycin. The NSG mice were anesthetized and 1×105 FRα⁺Luc2-2A-E2Crimson⁺KPCY2838c3 cells in 100 μL 1×PBS were i.p. implanted. After 10 days, the mice were randomized into four groups (n=5 each) and three groups were treated with doses of 1×106, 3×10⁶, or 10×10⁶ FRα-CAR⁺ T-cells. The fourth untreated group was used for negative control. The tumor growth was monitored every 3-4 days using i.p. injected 150 mg D-Luciferin per kg of mouse dissolved in 1×PBS. The luminescence imaging was performed in an AMI HTX Spectral instrument with a 60-see exposure. The data were quantified by analysis of the region-of-interest (ROI) using Aura Image software. The tumor luminescence is plotted as the mean±SEM of total flux (photons/s) against days after tumor implantation.
(10) In vivo validation of delivery function of the engineered T-cells (engineered for delivery function with NFAT-RE delivery system). The in vivo validation of the T-cell based delivery system was performed in mice at SRI International in accordance with the guidelines from the Institutional Animal Care and Use Committee (Approval #22001). 6-8-week-old female NOD.Cg-Prkde^{scid} Il2rg^{tmlWjl}/SzJ (NSG) mice were purchased from The Jackson Laboratory. After mandatory quarantine, the NSG mice were anesthetized and 2×10⁶ FRα⁺Luc2-2A-E2Crimson⁺ A2780cis cells in 100 μL 1×PBS were i.p. implanted. The tumor growth was monitored every 3-4 days for the next 12 days using i.p. injected 150 mg D-Luciferin per kg of mouse dissolved in 1×PBS. At 11 days after implantation, the mice were randomized into two groups (n=5 each). The two groups were then treated with 2×10⁶ primary T-cells engineered for delivery function (e.g., FRα-CAR with NFAT-RE inducible Nluc reporter) or the control T-cells (FRα-CAR only, e.g., without NFAT-RE inducible Nluc reporter) every day for 5 days. The bioluminescent reporter (Nluc) activity was determined by i.p. injection of the Nano-Glo® substrate (1:20 dilution of the substrate in 1×PBS, equivalent to 0.5 mg per kg of mouse) on days 0, 1, 2, 3, 4, and 5 after treatment. Imaging was performed in a IVIS Lumina X5 imaging system. The data was quantified by analysis of the ROI using Living Image software. The tumor luminescence is plotted as the mean±SEM of total flux (photons/s) against days after treatment.
(11) Statistical analysis. Results are expressed as an arithmetic mean±SD if not otherwise stated. For each run, each sample was measured in a technical replicate. Values of p<0.05 were considered to indicate statistical significance (represented as *p<0.05, ** p<0.01, *** p<0.001 and **** p<0.0001), as determined using ANOVA with Tukey's multiple comparison test, or as specified. Analyses were performed using Prism version 8.0 (GraphPad Software).

The various above described experiments used a variety of different plasmids to form the effector cells and controls. Table 3 below provides a listing of the plasmids and associated sequences.

TABLE 3
Listing of plasmids used in experimental embodiments

| FIGURE | GENE | SEQ ID NO |
|---|---|---|
| FIG. 7A | Anti-FRα CAR-28-BBζ (Control) | SEQ ID NO: 1 |
| FIG. 7B | Anti-FRα CAR-28-BBζ NLuc Biofactory | SEQ ID NO: 2 |
| FIG. 7C | Anti-FRα CAR-28ζ NLuc Biofactory | SEQ ID NO: 4 |
| FIG. 7D | Anti-FRα CAR-BBζ NLuc Biofactory | SEQ ID NO: 6 |
| FIG. 7E | Anti-FRα CAR-BBζ IFNβ Biofactory | SEQ ID NO: 8 |
| FIGS. 8C-8D | Anti-FRα CAR-28-BBζ NLuc Biofactory | SEQ ID NO: 2 |
| | pAdVAntage* | *commercially available, |
| | pMD2.G* | see Tables 1-2 |
| | psPAX2* | |
| FIGS. 8E-8F | Anti-FRα CAR-28-BBζ NLuc Biofactory | SEQ ID NO: 2 |
| | Anti-FRα CAR-28ζ NLuc Biofactory | SEQ ID NO: 4 |
| | Anti-FRα CAR-BBζ NLuc Biofactory | SEQ ID NO: 6 |
| | pAdVAntage* | *commercially available, |
| | pMD2.G* | see Tables 1-2 |
| | psPAX2* | |
| FIG. 8G | Anti-FRα CAR-28-BBζ NLuc Biofactory | SEQ ID NO: 2 |
| | pAdVAntage* | *commercially available, |
| | pMD2.G* | see Tables 1-2 |
| | psPAX2* | |
| FIG. 8H | Anti-FRα CAR-28-BBζ NLuc Biofactory | SEQ ID NO: 2 |
| | FRα-Luc2-E2Crimson | SEQ ID NO: 10 |
| | pAdVAntage* | *commercially available, |
| | pMD2.G* | see Tables 1-2 |
| | psPAX2* | |
| FIGS. 9B-9C | Anti-FRα CAR-28-BBζ NLuc Biofactory | SEQ ID NO: 2 |
| | No CAR NLuc Biofactory | SEQ ID NO: 11 |
| | Anti-FRα CAR- BBζ | SEQ ID NO: 12 |
| | FRα-Luc2-E2Crimson | SEQ ID NO: 10 |
| | MSLN-Luc2-E2Crimson | SEQ ID NO: 13 |
| | pAdVAntage* | *commercially available, |
| | pMD2.G* | see Tables 1-2 |
| | psPAX2* | |
| FIG. 10A | Anti-FRα CAR-BBζ IFNβ Biofactory | SEQ ID NO: 8 |
| | Anti-FRα CAR- BBζ | SEQ ID NO: 12 |
| | pAdVAntage* | *commercially available, |
| | pMD2.G* | see Tables 1-2 |
| | psPAX2* | |
| FIG. 10B | Anti-FRα CAR-BBζ IFNβ Biofactory | SEQ ID NO: 8 |
| | FRα-Luc2-E2Crimson | SEQ ID NO: 14 |
| | pAdVAntage* | *commercially available, |
| | pMD2.G* | see Tables 1-2 |
| | psPAX2* | |
| FIG. 10C | Anti-FRα CAR-BBζ IFNβ Biofactory | SEQ ID NO: 8 |
| | FRα-Luc2-E2Crimson | SEQ ID NO: 10 |
| | pAdVAntage* | *commercially available, |
| | pMD2.G* | see Tables 1-2 |
| | psPAX2* | |
| FIG. 10D | Anti-FRα CAR-BBζ IFNβ Biofactory | SEQ ID NO: 8 |
| | pAdVAntage* | *commercially available, |
| | pMD2.G* | see Tables 1-2 |
| | psPAX2* | |
| FIGS. 10F- | Anti-FRα CAR-BBζ IFNβ Biofactory | SEQ ID NO: 8 |
| 10H | Anti-FRα CAR- BBζ | SEQ ID NO: 12 |
| | FRα-Luc2-E2Crimson | SEQ ID NO: 10 |
| | pAdVAntage* | *commercially available, |
| | pMD2.G* | see Tables 1-2 |
| | psPAX2* | |
| FIG. 11 | Anti-FRα CAR-28-BBζ ( Control) | SEQ ID NO: 1 |
| | pAdVAntage* | *commercially available, |
| | pMD2.G* | see Tables 1-2 |
| | psPAX2* | |
| FIG. 15A | Anti-FRα CAR-28-BBζ NLuc Biofactory | SEQ ID NO: 2 |
| | pAdVAntage* | *commercially available, |
| | pMD2.G* | see Tables 1-2 |
| | psPAX2* | |
| FIG. 15B | Anti-FRα CAR-28-BBζ NLuc Biofactory | SEQ ID NO: 2 |
| | Anti-FRα CAR-28-BBζ (Control) | SEQ ID NO: 1 |
| | pAdVAntage* | *commercially available, |
| | pMD2.G* | see Tables 1-2 |
| | psPAX2* | |
| FIG. 15C | Anti-FRα CAR-28-BBζ NLuc Biofactory | SEQ ID NO: 2 |
| | pMD2-RD114A | SEQ ID NO: 15 |
| | pAdVAntage* | *commercially available, |
| | pMD2.G* | see Tables 1-2 |
| | psPAX2* | |
| FIG. 15D | Anti-FRα CAR-28-BBζ NLuc Biofactory | SEQ ID NO: 2 |
| | pAdVAntage* | *commercially available, |
| | pMD2.G* | see Tables 1-2 |
| | psPAX2* | |
| FIGS. 16A- | Anti-FRα CAR-28-BBζ NLuc Biofactory | SEQ ID NO: 2 |
| 16C | pAdVAntage* | *commercially available, |
| | pMD2.G* | see Tables 1-2 |
| | psPAX2* | |
| FIG. 16D | Anti-FRα CAR-28-BBζ NLuc Biofactory | SEQ ID NO: 2 |
| | FRα-Luc2-E2Crimson | SEQ ID NO: 10 |
| | pAdVAntage* | *commercially available, |
| | pMD2.G* | see Tables 1-2 |
| | psPAX2* | |
| FIG. 16E | Anti-FRα CAR-28-BBζ NLuc Biofactory | SEQ ID NO: 2 |
| | pAdVAntage* | *commercially available, |
| | pMD2.G* | see Tables 1-2 |
| | psPAX2* | |
| FIG. 17A | Anti-FRα CAR-28-BBζ NLuc Biofactory | SEQ ID NO: 2 |
| | FRα-Luc2-E2Crimson | SEQ ID NO: 10 |
| | pAdVAntage* | *commercially available, |
| | pMD2.G* | see Tables 1-2 |
| | psPAX2* | |
| FIG. 17B | Anti-FRα CAR-28-BBζ NLuc Biofactory | SEQ ID NO: 2 |
| | FRα-Luc2-E2Crimson | SEQ ID NO: 16 |
| | pAdVAntage* | *commercially available, |
| | pMD2.G* | see Tables 1-2 |
| | psPAX2* | |
| FIG. 17C | Anti-FRα CAR-28-BBζ (Control) | SEQ ID NO: 1 |
| | FRα-Luc2-E2Crimson | SEQ ID NO: 16 |
| | pAdVAntage* | *commercially available, |
| | pMD2.G* | see Tables 1-2 |
| | psPAX2* | |
| FIGS. 17E- | Anti-FRα CAR-28-BBζ NLuc Biofactory | SEQ ID NO: 2 |
| 17F | Anti-FRα CAR-28-BBζ (Control) | SEQ ID NO: 1 |
| | FRα-Luc2-E2Crimson | SEQ ID NO: 16 |
| | pAdVAntage* | *commercially available, |
| | pMD2.G* | see Tables 1-2 |
| | psPAX2* | |
| FIGS. 19A- | Anti-FRα CAR-28-BBζ NLuc Biofactory | SEQ ID NO: 2 |
| 19D | Anti-FRα CAR-28-BBζ (Control) | SEQ ID NO: 1 |
| FIGS. 20A- | Anti-FRα CAR-28-BBζ NLuc Biofactory | SEQ ID NO: 2 |
| 20B | pAdVAntage* | *commercially available, |
| | pMD2.G* | see Tables 1-2 |
| | psPAX2* | |
| FIG. 21 | Anti-FRα CAR-28-BBζ NLuc Biofactory | SEQ ID NO: 2 |
| | pAdVAntage* | *commercially available, |
| | pMD2.G* | see Tables 1-2 |
| | psPAX2* | |
| FIG. 22A | Anti-FRα CAR-28-BBζ (Control) | SEQ ID NO: 1 |
| | FRα-Luc2-E2Crimson | SEQ ID NO: 16 |
| | Luc2-E2Crimson | SEQ ID NO: 17 |
| | pAdVAntage* | *commercially available, |
| | pMD2.G* | see Tables 1-2 |
| | psPAX2* | |
| FIG. 22B | Anti-FRα CAR-28-BBζ (Control) | SEQ ID NO: 1 |
| | FRα-Luc2-E2Crimson | SEQ ID NO: 10 |
| | Luc2-E2Crimson | SEQ ID NO: 18 |
| | pAdVAntage* | *commercially available, |
| | pMD2.G* | see Tables 1-2 |
| | psPAX2* | |
| FIG. 22C | No CAR NLuc Biofactory | SEQ ID NO: 11 |
| | FRα-Luc2-E2Crimson | SEQ ID NO: 16 |
| | FRα-Luc2-E2Crimson | SEQ ID NO: 10 |
| | Luc2-E2Crimson | SEQ ID NO: 17 |
| | Luc2-E2Crimson | SEQ ID NO: 18 |
| | pAdVAntage* | \grave{ }*commercially available, |
| | pMD2.G* | see Tables 1-2 |
| | psPAX2* | |

Various genetically engineered effector cells were formed in experimental embodiments that capitalized on the biology of CD4 T-cells and transformed the CD4 T-cells into a cell-based platform that can assess the disease burden and mount a proportional response by expressing engineered proteins precisely at the disease site. The helper CD4 T-cell was demonstrated to offer a clear competitive advantage over the killer CD8 T-cell or a combined pool of CD4 and CD8 T-cells (e.g., pan CD3 T-cells) when engineered for cell-based effector cells for delivering proteins. Overall, the correctly engineered CD4 T-cells offer to bridge two gaps that may prevent the clinical adoption of this technology, e.g., scaling up the cell production to generate a clinical dose and increased synthesis of the therapeutic protein from the cell so as to reduce the size of the clinical dose required to produce the desired effect.

In various embodiments, a scalable process for forming or manufacturing lentiviral vector particles was used at a high titer to support the transduction of primary T-cells with large genetic inserts and a simple and efficient process to engineer primary T-cells, as further described below. In experimental embodiments, CD4 T-cells were compared with CD8 T-cells, when engineered for the delivery function and favor CD4 T-cells for multiple reasons. For example, the CD4 T-cells engineered with a CAR that has 4-1BB (intracellular domain but not the CD28 intracellular domain, compared to a similarly engineered CD8 CAR T-cell, exhibits around thirty-fold cumulative improved performance (around three-fold transduction, around two-fold expansion, around five-fold activity). When starting with pan CD3 T-cells, which are the population of combined CD4 and CD8 T-cells, somewhat surprising, the final population after cell expansion consisted mainly of CD8 T-cells.

The non-cytolytic CD4 T-cells were experimentally confirmed as being an appropriate phenotype for performing this function. The low cytolysis of CD4 T-cell delivery system offers a substantial advantage, e.g., the maximum tolerated dose of the CD4 T-cell engineered for drug delivery can be high without exposure to the healthy tissues. The CD4 T-cells are believed to be advantageous over the CD8 T-cells due to the NFAT-based transcriptional machinery that is more productive in CD4 T-cells. Further, CD4 T-cells produce more Th1 cytokines and proliferate faster than CD8 T-cells. Compared to pan CD3 CAR T-cells with a non-curable dose, the same number of T-cells with half from each subset (CD4, CD8) more effectively treats tumors. Helper CD4 T-cells induce stemness in the CD8 T-cells that then persists longer and increases the memory T-cell pool for improved treatment outcome. Unlike CD8 T-cells, the CD4 T-cells extravasate into cold tumors and recruit other CD8 T-cells. The CD4 T-cells also assist vascular normalization, attenuate hypoxia, and reduce metastasis. CD4 T-cells have been found to persist for more than a decade. In fact, when passaged in vivo, CD4 T-cells can outlive the host mouse specie by four times and expand at least 1040-fold.

The cell-mediated drug delivery system of experimental embodiments can positively impact various medical domains that require precise spatiotemporal drug administration, such as for leveraging it for solid tumors and viral infections. Building upon experiments with cell-based delivery of IFNβ for targeting solid tumors and viral infections, experiments were directed to assessing the antitumor efficacy by focusing the localized delivery of IFNβ through the CD4 T-cell based effector cells. The resulting data showed that in situ delivery of IFNβ using the CD4 T-cell-based effector cell substantially outperformed the direct administration of rhIFNβ dose of 0.25 μg (70,000 IU) per day, a tolerable dose used in mice. It was further showed that daily i.p. delivery of 0.25 μg rhIFNβ (70,000 IU) for 6 days (a total combined dose of 420,000 IU) failed to demonstrate any significant therapeutic advantage. This is in consensus with previous reports where daily i.p. administration of rhIFNβ (10,000 IU) for 23 days (a total combined dose of 230,000 IU) failed to elicit any therapeutic response. In fact, the calculations show (see above related to FIG. 10A calculations) that the IFNβ dosage delivered by CD4 T-cells was around 300-fold less than the directly administered rhIFNβ, yet more effective. The increased therapeutic effect at reduced equivalent dose shows the potential of the cell-based platform to minimize undesired side-effects to the healthy tissues thereby increasing the Maximum Recommended Starting Dose during subsequent human trials. Such effector cells can continue the development for overcoming the two major issues that have been observed with systemic IFNβ treatments-severe toxicities to healthy issues and immunosuppression in the local tumor microenvironment; and use it to synergize with other antitumor agents such as chemotherapies, radiotherapies, antibody-based immunotherapies, as well as bridge the innate and adaptive immune responses.

The CD4 T-cell-based technology acts as a zero-order delivery system, providing a promising solution to the challenges posed by the first-order drug delivery systems with synthetic carriers. Unlike first-order systems, it focusses the concentration of therapeutic biologics at the disease site proportionate to the disease mass, increasing efficacy while reducing concentration in healthy tissues, improving safety. Furthermore, the long-term persistence of CD4 T-cells in the body, which can last for more than a decade, holds great promise in significantly reducing the frequency of re-infusions required. This will not only improve patient compliance but also, over the long term, alleviate the burden on the healthcare system and streamline treatment procedures.

In the additionally described experiments, primary T-cells were used to develop a cell-based platform that can be used for site-specific delivery of protein-based drugs. The platform delivery system utilizes the T-cell's activation machinery for in situ synthesis, so that the cell-mediated synthesis of desired proteins is proportionate to the disease burden. The site-specific and proportionate synthesis of desired biologics offers the potential to overcome morbidity issues that can arise from excess systemic infusion of such drugs, and prevents the development of resistance to these drugs when used in lower amounts Additional experiments were directed to assessment of AVIs and LRAs to improve the lentivector transduction yield and increase the starting cell number so as to shorten dose-manufacturing time, thereby improving the affordability of T-cell therapies. The use of these additives, such as AVIs and LRAs, can also reduce cell exhaustion by decondensing the chromatin structure.

Although first-order drug-delivery systems (e.g., liposomes, nano-carries, dendrimers, hydrogels, microparticles) offer a controlled release of drugs, their application is still limited by their short half-life in vivo requirement for multiple infusions, and potential toxicity due to their systemic presence. The above described T-cell-based effector cells represents a substantive departure from this status quo. This is because T-cells chemotactically extravasate through multiple solid tissues to the disease sites and engage with the target cells through the antigen-specific CAR. At the single-cell level, this cues the activation pathway in a binary (on/off) event independent of the antigen density on the surface of the disease cell, and executes a parallel program resulting in clonal expansion of the activated T-cells. The integrated effect is a clonal CAR T-cell population proportionate to the number of target cells. The T-cell-based drug delivery system is engineered to leverage this biology of the T-cell. T-cells migrate to disease sites with cellular resolution and, upon recognizing the target cells with molecular specificity, can synthesize protein-based biologics proportionate to the disease burden. It is therefore a living cell-based in vivo vector engineered into a stable zero-order drug delivery system. Unlike the first-order drug delivery systems, it can enable sustained in situ production of complex biologic drugs for executing a broad range of effector functions.

Additionally, the first-order drug-delivery approaches are primarily based on synthetic materials and are thus rapidly cleared by the mononuclear phagocyte system. In contrast, the cell-based system utilizing T-cells have been found to persist in vivo for more than a decade. In fact, recent findings in mice concluded that the primary T-cell, when passaged in vivo in new mice, can last four times longer than the lifespan of the host species and expand at least 10⁴⁰-fold. This obviates the need for re-dosing even in case of relapse. Therefore, while the potential to target the basal expression of the target antigen exists, as seen in the case of CAR T-cells, the T-cell-based delivery platform presents a pioneering and universal technology. It facilitates the delivery of intricate biologics over extended periods without the need for multiple infusions. As a result, this platform technology opens new horizons for treating a variety of diseases.

Although specific examples have been illustrated and described herein, a variety of alternate and/or equivalent implementations can be substituted for the specific examples shown and described without departing from the scope of the present disclosure. This application is intended to cover any adaptations or variations of the specific examples discussed herein.

1. A genetically engineered effector cell comprising an isolated CD4 T-cell carrying an exogenous polynucleotide sequence that includes, in operative association:
a receptor element that encodes a chimeric antigen receptor (CAR) including an extracellular antigen binding domain operably linked to a transmembrane domain, and an intracellular signaling domain, wherein the extracellular antigen binding domain recognizes an antigen on a surface of a target cell;
an actuator element that encodes a transcription factor binding site that upregulates synthesis of an effector protein in response to the extracellular antigen binding domain of the CAR binding to the antigen of the target cell; and
an effector element that encodes the effector protein, wherein, in response to the extracellular antigen binding domain of the CAR binding to the antigen of the target cell, the genetically engineered effector cell is configured to activate and, to synthesize and secrete the effector protein.

2. The genetically engineered effector cell of claim 1, wherein the effector element further encodes a signal peptide operably linked to the effector protein, the signal peptide being non-native to the effector protein.

3. The genetically engineered effector cell of claim 1, wherein the genetically engineered effector cell is configured to synthesize and secrete an amount of the effector protein as a function of an amount of the target cell present in a sample or in situ.

4. The genetically engineered effector cell of claim 1, wherein the CAR is configured to cause a rise in calcium in response to the extracellular antigen binding domain binding to the antigen of the target cell and the transcription factor binding site is configured to bind to a transcription factor protein that is triggered by the rise in calcium and is translocated into the nucleus of the genetically engineered effector cell.

5. The genetically engineered effector cell of claim 1, wherein the intracellular signaling domain is selected from the group consisting of:
an intracellular signaling portion of a 4-1BB, an intracellular signaling portion of a CD3 zeta, and a combination thereof.

6. The genetically engineered effector cell of claim 1, wherein the intracellular signaling domain does not include an intracellular signaling portion of CD28.

7. The genetically engineered effector cell of claim 1, wherein the transcription factor binding site is selected from the group consisting of:
a nuclear factor of activated T-cell (NFAT) response element, a serum response element (SRE), a cyclic AMP response element (CRE), and a combination thereof.

8. The genetically engineered effector cell of claim 1, wherein the effector protein is selected from the group consisting of:
a detectable reporter protein, a therapeutic protein, a downstream signaling protein, and a combination thereof.

9. The genetically engineered effector cell of claim 1, wherein the exogenous polynucleotide sequence includes, in operative association, the receptor element, the actuator element, and the effector element on a single construct.

10. The genetically engineered effector cell of claim 1, wherein the transmembrane domain is selected from the group consisting of:
T-cell receptor α or β chain, a CD3ζ chain, CD28, CD3ε, CD45, CD4, CD5, CD8, CD9, CD16, CD22, CD28, CD33, CD37, CD64, CD80, CD86, CD134, CD137, ICOS, CD154, and a GITR.

11. A single construct configured to form a genetically engineered effector cell with an isolated CD4 T-cell for secretion of an effector protein upon recognition of an antigen on a surface of a target cell, the single construct comprising an exogenous polynucleotide sequence including, in operative association:
a receptor element that encodes a chimeric antigen receptor (CAR) including an extracellular antigen binding domain operably linked to a transmembrane domain, and an intracellular signaling domain, wherein the extracellular antigen binding domain recognizes an antigen on a surface of a target cell;
an actuator element that encodes a transcription factor binding site that upregulates synthesis of an effector protein in response to the extracellular antigen binding domain of the CAR binding to the antigen of the target cell; and
an effector element that encodes the effector protein, wherein, in response to the extracellular antigen binding domain of the CAR binding to the antigen of the target cell, the genetically engineered effector cell is configured to activate and, to synthesize and secrete the effector protein.

12. (canceled)

13. The single construct of claim 11, wherein the single construct is carried by a viral vector or a non-viral carrier.

14. The single construct of claim 11, wherein the intracellular signaling domain includes each of:
an intracellular signaling portion of a 4-1BB and an intracellular signaling portion of a CD3 zeta and wherein the intracellular signaling domain does not include an intracellular signaling portion of CD28.

15. (canceled)

16. The single construct of claim 11, wherein:
the transcription factor binding site is selected from the group consisting of:
a nuclear factor of activated T-cell (NFAT) response element, a serum response element (SRE), a cyclic AMP response element (CRE), and a combination thereof; and
the transmembrane domain is selected from the group consisting of:
T-cell receptor α or β chain, a CD3ζ chain, CD28, CD3ε, CD45, CD4, CD5, CD8, CD9, CD16, CD22, CD28, CD33, CD37, CD64, CD80, CD86, CD134, CD137, ICOS, CD154, and a GITR.

17. (canceled)

18. The single construct of claim 11, wherein the exogenous polynucleotide sequence includes a sequence with at least 80% sequence identity to a sequence selected from SEQ ID NOs: 1-20.

19. A population of genetically engineered effector cells, each of the genetically engineered effector cells of the population comprising an isolated CD4 T-cell carrying an exogenous polynucleotide sequence that includes an actuator element bound to an effector element bound to a receptor element, wherein:
a receptor element that encodes a chimeric antigen receptor (CAR) including an extracellular antigen binding domain operably linked to a transmembrane domain, and an intracellular signaling domain, wherein the extracellular antigen binding domain recognizes an antigen on a surface of a target cell;
an actuator element that encodes a transcription factor binding site that upregulates synthesis of an effector protein in response to the extracellular antigen binding domain of the CAR binding to the antigen of the target cell; and
an effector element that encodes the effector protein, wherein, in response to the extracellular antigen binding domain of the CAR binding to the antigen of the target cell, the population of genetically engineered effector cells is configured to activate and, to synthesize and secrete the effector protein.

20. The population of genetically engineered effector cells of claim 19, wherein the population of engineered effector cells are configured to activate and, in response, to synthesize and secrete a calibrated amount of the effector protein based on a presence of the target cell, the calibrated amount of the effector protein being a function of an amount of the target cell present in a plurality of cells or in a sample.

21. The population of genetically engineered effector cells of claim 19, wherein each effector element further encodes a signal peptide operably linked to the effector protein.

22. The population of genetically engineered effector cells of claim 19, wherein the intracellular signaling domain is selected from the group consisting of:
an intracellular signaling portion of a 4-1BB, an intracellular signaling portion of a CD3 zeta, and a combination thereof, and wherein the intracellular signaling domain does not include an intracellular signaling portion of CD28.

23. (canceled)

24. The population of genetically engineered effector cells of claim 19, wherein:
the transcription factor binding site is selected from the group consisting of:
a nuclear factor of activated T-cell (NFAT) response element, a serum response element (SRE), a cyclic AMP response element (CRE), and a combination thereof; and
the transmembrane domain is selected from the group consisting of:
T-cell receptor α or β chain, a CD32 chain, CD28, CD3ε, CD45, CD4, CD5, CD8, CD9, CD16, CD22, CD28, CD33, CD37, CD64, CD80, CD86, CD134, CD137, ICOS, CD154, and a GITR.

25-81. (canceled)


SEQUENCE LISTING (20 sequences)

SEQ ID NO: 1 - DNA, 8909 bp, synthetic construct
  source 1..8909: mol_type = other DNA
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatgctagcgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactagtgccaccatggactttcaagtgcagatcttcagctttctgctgatctctgctagtgtcatcatgtctgaagtgcaactggtggaatctggaggtggagtggtgcaaccaggtaggtcactcaggctgtcttgctcagcctctggcttcacattctcaggatatggtctgagctgggtcagacaagcaccaggcaagggactggaatgggtcgctatgatcagcagtggaggatcttacacctactatgcagattctgtcaagggtaggtttgcaatctccagagacaatgccaagaacaccctgttcctgcagatggacagcctcagacctgaagatactggagtttacttctgtgctaggcacggagacgatccagcctggtttgcctactggggccaaggaactccagtgacagtctcttctgccagcactaagggtcctagcgtgtttccactggctcccagctccaagtcaacaagtggaggtacagcagctctcggatgtctggtgaaagattacttccctgagcctgtcacagtcagttctggaggtggaggcagtggaggtggtggatctggaggaggagggtctgacatccaactgacacagtcaccatcttcactgtctgccagcgtgggagacagggtcaccattacatgctctgtttcctcttccatatcatcaaacaatctccactggtatcaacagaagcctggtaaggctcccaaaccctggatctatggcacaagcaacctcgcatctggtgtgccatcacgcttctcaggctctgggagtggcacagactacacctttactatctcaagcctgcaacctgaggacattgctacctactattgccagcagtggtcttcatacccttacatgtacacctttggtcagggaactaaggtggaaatcaagagggaaatcgagcagaagctgatttccgaagaggatctcaatggagtgactgtcagcagcgctctgtctaactccatcatgtacttctcacacttcgtgccagtgttcctccctgctaaacccaccacaactccagcacctagacctcccactccagcaccaactattgcatcccagcctctctccctcagaccagaagcatgcagacctgcagcaggtggagctgtgcacacaagaggtctggaccctttctgggtcctcgtggtggtgggtggagtcctggcatgttacagcctcctggtcaccgtggcattcatcatcttctgggtgagatctaagaggagcagactgctgcactctgattacatgaacatgacacccagaagacctggtcccaccagaaagcactaccaaccctacgcaccaccaagagactttgctgcatacagaagtaagagagggaggaagaagctgctgtacatcttcaaacaaccattcatgaggcctgtgcagacaacccaggaggaagacggatgctcatgcaggttcccagaggaggaggaaggaggttgcgaactcctggagagggtcaagttctcaaggagtgcagatgctccagcctatcaacagggtcagaaccaactgtacaacgagctgaatctcggaagaagagaggagtacgatgtgctggataagagaagaggcagggacccagagatgggtgggaaacccagaagaaagaatcctcaagagggactgtacaatgagctgcagaaggataagatggctgaggcatactcagagatcggtatgaagggagagaggagaagaggcaaaggtcatgatggtctgtaccaaggtctgtccacagcaacaaaggatacatatgatgctctgcacatgcaggcactcccaccacggggttccggagtgaaacagactttgaactttgaccttctcaagttggctggagacgtcgagtccaaccctggtcccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgattaattaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 2 - DNA, 10489 bp, synthetic construct
  source 1..10489: mol_type = other DNA
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatgctagcggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtagatctagactctagagggtatataatggaagctcgacttccagcttggcattccggtactgttggtaaaaagcttggcaatccggtactgttggtaaaggatccgccaccatgcctgccatgaagattgagtgccgcatcacgggaaccctgaacggagtggagtttgagctggtcggaggtggagaagggactcctgagcagggacgtatgaccaacaagatgaagtctaccaagggcgccttgaccttctccccctaccttctctctcatgtcatgggatacgggttctaccactttggtacctatcccagtgggtatgagaatcccttcctgcatgccatcaacaacggggggtacaccaacaccaggattgagaagtatgaggatggaggagttcttcatgttagctttagctacagatatgaagcaggcagggtgattggggatttcaaggttgtcgggacaggattccctgaggacagtgtgatcttcaccgacaagatcatccggtccaatgctaccgtggagcacttgcacccaatgggagacaacgttcttgtgggctccttcgcgagaaccttttccctgagggatggaggctactactcatttgtggttgacagccacatgcacttcaagagtgccatccacccatccatcctccagaacggggggcccatgtttgccttcaggagagttgaggaacttcactccaacactgaacttggcattgtagagtatcaacatgccttcaagactcccatagcatttgctggaagcggagctactaacttcagcctgctgaagcaggctggagacgtggaggagaaccctggacctatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaagcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactagtgccaccatggactttcaagtgcagatcttcagctttctgctgatctctgctagtgtcatcatgtctgaagtgcaactggtggaatctggaggtggagtggtgcaaccaggtaggtcactcaggctgtcttgctcagcctctggcttcacattctcaggatatggtctgagctgggtcagacaagcaccaggcaagggactggaatgggtcgctatgatcagcagtggaggatcttacacctactatgcagattctgtcaagggtaggtttgcaatctccagagacaatgccaagaacaccctgttcctgcagatggacagcctcagacctgaagatactggagtttacttctgtgctaggcacggagacgatccagcctggtttgcctactggggccaaggaactccagtgacagtctcttctgccagcactaagggtcctagcgtgtttccactggctcccagctccaagtcaacaagtggaggtacagcagctctcggatgtctggtgaaagattacttccctgagcctgtcacagtcagttctggaggtggaggcagtggaggtggtggatctggaggaggagggtctgacatccaactgacacagtcaccatcttcactgtctgccagcgtgggagacagggtcaccattacatgctctgtttcctcttccatatcatcaaacaatctccactggtatcaacagaagcctggtaaggctcccaaaccctggatctatggcacaagcaacctcgcatctggtgtgccatcacgcttctcaggctctgggagtggcacagactacacctttactatctcaagcctgcaacctgaggacattgctacctactattgccagcagtggtcttcatacccttacatgtacacctttggtcagggaactaaggtggaaatcaagagggaaatcgagcagaagctgatttccgaagaggatctcaatggagtgactgtcagcagcgctctgtctaactccatcatgtacttctcacacttcgtgccagtgttcctccctgctaaacccaccacaactccagcacctagacctcccactccagcaccaactattgcatcccagcctctctccctcagaccagaagcatgcagacctgcagcaggtggagctgtgcacacaagaggtctggaccctttctgggtcctcgtggtggtgggtggagtcctggcatgttacagcctcctggtcaccgtggcattcatcatcttctgggtgagatctaagaggagcagactgctgcactctgattacatgaacatgacacccagaagacctggtcccaccagaaagcactaccaaccctacgcaccaccaagagactttgctgcatacagaagtaagagagggaggaagaagctgctgtacatcttcaaacaaccattcatgaggcctgtgcagacaacccaggaggaagacggatgctcatgcaggttcccagaggaggaggaaggaggttgcgaactcctggagagggtcaagttctcaaggagtgcagatgctccagcctatcaacagggtcagaaccaactgtacaacgagctgaatctcggaagaagagaggagtacgatgtgctggataagagaagaggcagggacccagagatgggtgggaaacccagaagaaagaatcctcaagagggactgtacaatgagctgcagaaggataagatggctgaggcatactcagagatcggtatgaagggagagaggagaagaggcaaaggtcatgatggtctgtaccaaggtctgtccacagcaacaaaggatacatatgatgctctgcacatgcaggcactcccaccacggggttccggagtgaaacagactttgaactttgaccttctcaagttggctggagacgtcgagtccaaccctggtcccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgattaattaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 3 - DNA, 672 bp, synthetic construct
  source 1..672: mol_type = other DNA
ttctgggtcctcgtggtggtgggtggagtcctggcatgttacagcctcctggtcaccgtggcattcatcatcttctgggtgagatctaagaggagcagactgctgcactctgattacatgaacatgacacccagaagacctggtcccaccagaaagcactaccaaccctacgcaccaccaagagactttgctgcatacagaagtaagagagggaggaagaagctgctgtacatcttcaaacaaccattcatgaggcctgtgcagacaacccaggaggaagacggatgctcatgcaggttcccagaggaggaggaaggaggttgcgaactcctggagagggtcaagttctcaaggagtgcagatgctccagcctatcaacagggtcagaaccaactgtacaacgagctgaatctcggaagaagagaggagtacgatgtgctggataagagaagaggcagggacccagagatgggtgggaaacccagaagaaagaatcctcaagagggactgtacaatgagctgcagaaggataagatggctgaggcatactcagagatcggtatgaagggagagaggagaagaggcaaaggtcatgatggtctgtaccaaggtctgtccacagcaacaaaggatacatatgatgctctgcacatgcaggcactcccaccacgg

SEQ ID NO: 4 - DNA, 10363 bp, synthetic construct
  source 1..10363: mol_type = other DNA
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatgctagcggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtagatctagactctagagggtatataatggaagctcgacttccagcttggcattccggtactgttggtaaaaagcttggcaatccggtactgttggtaaaggatccgccaccatgcctgccatgaagattgagtgccgcatcacgggaaccctgaacggagtggagtttgagctggtcggaggtggagaagggactcctgagcagggacgtatgaccaacaagatgaagtctaccaagggcgccttgaccttctccccctaccttctctctcatgtcatgggatacgggttctaccactttggtacctatcccagtgggtatgagaatcccttcctgcatgccatcaacaacggggggtacaccaacaccaggattgagaagtatgaggatggaggagttcttcatgttagctttagctacagatatgaagcaggcagggtgattggggatttcaaggttgtcgggacaggattccctgaggacagtgtgatcttcaccgacaagatcatccggtccaatgctaccgtggagcacttgcacccaatgggagacaacgttcttgtgggctccttcgcgagaaccttttccctgagggatggaggctactactcatttgtggttgacagccacatgcacttcaagagtgccatccacccatccatcctccagaacggggggcccatgtttgccttcaggagagttgaggaacttcactccaacactgaacttggcattgtagagtatcaacatgccttcaagactcccatagcatttgctggaagcggagctactaacttcagcctgctgaagcaggctggagacgtggaggagaaccctggacctatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaagcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactagtgccaccatggactttcaagtgcagatcttcagctttctgctgatctctgctagtgtcatcatgtctgaagtgcaactggtggaatctggaggtggagtggtgcaaccaggtaggtcactcaggctgtcttgctcagcctctggcttcacattctcaggatatggtctgagctgggtcagacaagcaccaggcaagggactggaatgggtcgctatgatcagcagtggaggatcttacacctactatgcagattctgtcaagggtaggtttgcaatctccagagacaatgccaagaacaccctgttcctgcagatggacagcctcagacctgaagatactggagtttacttctgtgctaggcacggagacgatccagcctggtttgcctactggggccaaggaactccagtgacagtctcttctgccagcactaagggtcctagcgtgtttccactggctcccagctccaagtcaacaagtggaggtacagcagctctcggatgtctggtgaaagattacttccctgagcctgtcacagtcagttctggaggtggaggcagtggaggtggtggatctggaggaggagggtctgacatccaactgacacagtcaccatcttcactgtctgccagcgtgggagacagggtcaccattacatgctctgtttcctcttccatatcatcaaacaatctccactggtatcaacagaagcctggtaaggctcccaaaccctggatctatggcacaagcaacctcgcatctggtgtgccatcacgcttctcaggctctgggagtggcacagactacacctttactatctcaagcctgcaacctgaggacattgctacctactattgccagcagtggtcttcatacccttacatgtacacctttggtcagggaactaaggtggaaatcaagagggaaatcgagcagaagctgatttccgaagaggatctcaatggagtgactgtcagcagcgctctgtctaactccatcatgtacttctcacacttcgtgccagtgttcctccctgctaaacccaccacaactccagcacctagacctcccactccagcaccaactattgcatcccagcctctctccctcagaccagaagcatgcagacctgcagcaggtggagctgtgcacacaagaggtctggaccctttctgggtcctcgtggtggtgggtggagtcctggcatgttacagcctcctggtcaccgtggcattcatcatcttctgggtgagatctaagaggagcagactgctgcactctgattacatgaacatgacacccagaagacctggtcccaccagaaagcactaccaaccctacgcaccaccaagagactttgctgcatacagaagtctcgagagggtcaagttctcaaggagtgcagatgctccagcctatcaacagggtcagaaccaactgtacaacgagctgaatctcggaagaagagaggagtacgatgtgctggataagagaagaggcagggacccagagatgggtgggaaacccagaagaaagaatcctcaagagggactgtacaatgagctgcagaaggataagatggctgaggcatactcagagatcggtatgaagggagagaggagaagaggcaaaggtcatgatggtctgtaccaaggtctgtccacagcaacaaaggatacatatgatgctctgcacatgcaggcactcccaccacggggttccggagtgaaacagactttgaactttgaccttctcaagttggctggagacgtcgagtccaaccctggtcccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgattaattaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 5 - DNA, 546 bp, synthetic construct
  source 1..546: mol_type = other DNA
ttctgggtcctcgtggtggtgggtggagtcctggcatgttacagcctcctggtcaccgtggcattcatcatcttctgggtgagatctaagaggagcagactgctgcactctgattacatgaacatgacacccagaagacctggtcccaccagaaagcactaccaaccctacgcaccaccaagagactttgctgcatacagaagtctcgagagggtcaagttctcaaggagtgcagatgctccagcctatcaacagggtcagaaccaactgtacaacgagctgaatctcggaagaagagaggagtacgatgtgctggataagagaagaggcagggacccagagatgggtgggaaacccagaagaaagaatcctcaagagggactgtacaatgagctgcagaaggataagatggctgaggcatactcagagatcggtatgaagggagagaggagaagaggcaaaggtcatgatggtctgtaccaaggtctgtccacagcaacaaaggatacatatgatgctctgcacatgcaggcactcccaccacgg

SEQ ID NO: 6 - DNA, 10363 bp, synthetic construct
  source 1..10363: mol_type = other DNA
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatgctagcggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtagatctagactctagagggtatataatggaagctcgacttccagcttggcattccggtactgttggtaaaaagcttggcaatccggtactgttggtaaaggatccgccaccatgcctgccatgaagattgagtgccgcatcacgggaaccctgaacggagtggagtttgagctggtcggaggtggagaagggactcctgagcagggacgtatgaccaacaagatgaagtctaccaagggcgccttgaccttctccccctaccttctctctcatgtcatgggatacgggttctaccactttggtacctatcccagtgggtatgagaatcccttcctgcatgccatcaacaacggggggtacaccaacaccaggattgagaagtatgaggatggaggagttcttcatgttagctttagctacagatatgaagcaggcagggtgattggggatttcaaggttgtcgggacaggattccctgaggacagtgtgatcttcaccgacaagatcatccggtccaatgctaccgtggagcacttgcacccaatgggagacaacgttcttgtgggctccttcgcgagaaccttttccctgagggatggaggctactactcatttgtggttgacagccacatgcacttcaagagtgccatccacccatccatcctccagaacggggggcccatgtttgccttcaggagagttgaggaacttcactccaacactgaacttggcattgtagagtatcaacatgccttcaagactcccatagcatttgctggaagcggagctactaacttcagcctgctgaagcaggctggagacgtggaggagaaccctggacctatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaagcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactagtgccaccatggactttcaagtgcagatcttcagctttctgctgatctctgctagtgtcatcatgtctgaagtgcaactggtggaatctggaggtggagtggtgcaaccaggtaggtcactcaggctgtcttgctcagcctctggcttcacattctcaggatatggtctgagctgggtcagacaagcaccaggcaagggactggaatgggtcgctatgatcagcagtggaggatcttacacctactatgcagattctgtcaagggtaggtttgcaatctccagagacaatgccaagaacaccctgttcctgcagatggacagcctcagacctgaagatactggagtttacttctgtgctaggcacggagacgatccagcctggtttgcctactggggccaaggaactccagtgacagtctcttctgccagcactaagggtcctagcgtgtttccactggctcccagctccaagtcaacaagtggaggtacagcagctctcggatgtctggtgaaagattacttccctgagcctgtcacagtcagttctggaggtggaggcagtggaggtggtggatctggaggaggagggtctgacatccaactgacacagtcaccatcttcactgtctgccagcgtgggagacagggtcaccattacatgctctgtttcctcttccatatcatcaaacaatctccactggtatcaacagaagcctggtaaggctcccaaaccctggatctatggcacaagcaacctcgcatctggtgtgccatcacgcttctcaggctctgggagtggcacagactacacctttactatctcaagcctgcaacctgaggacattgctacctactattgccagcagtggtcttcatacccttacatgtacacctttggtcagggaactaaggtggaaatcaagagggaaatcgagcagaagctgatttccgaagaggatctcaatggagtgactgtcagcagcgctctgtctaactccatcatgtacttctcacacttcgtgccagtgttcctccctgctaaacccaccacaactccagcacctagacctcccactccagcaccaactattgcatcccagcctctctccctcagaccagaagcatgcagacctgcagcaggtggagctgtgcacacaagaggtctggaccctttctgggtcctcgtggtggtgggtggagtcctggcatgttacagcctcctggtcaccgtggcattcatcatcttctggaagagagggaggaagaagctgctgtacatcttcaaacaaccattcatgaggcctgtgcagacaacccaggaggaagacggatgctcatgcaggttcccagaggaggaggaaggaggttgcgaactcctggagagggtcaagttctcaaggagtgcagatgctccagcctatcaacagggtcagaaccaactgtacaacgagctgaatctcggaagaagagaggagtacgatgtgctggataagagaagaggcagggacccagagatgggtgggaaacccagaagaaagaatcctcaagagggactgtacaatgagctgcagaaggataagatggctgaggcatactcagagatcggtatgaagggagagaggagaagaggcaaaggtcatgatggtctgtaccaaggtctgtccacagcaacaaaggatacatatgatgctctgcacatgcaggcactcccaccacggggttccggagtgaaacagactttgaactttgaccttctcaagttggctggagacgtcgagtccaaccctggtcccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgattaattaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 7 - DNA, 468 bp, synthetic construct
  source 1..468: mol_type = other DNA
aagagagggaggaagaagctgctgtacatcttcaaacaaccattcatgaggcctgtgcagacaacccaggaggaagacggatgctcatgcaggttcccagaggaggaggaaggaggttgcgaactcctggagagggtcaagttctcaaggagtgcagatgctccagcctatcaacagggtcagaaccaactgtacaacgagctgaatctcggaagaagagaggagtacgatgtgctggataagagaagaggcagggacccagagatgggtgggaaacccagaagaaagaatcctcaagagggactgtacaatgagctgcagaaggataagatggctgaggcatactcagagatcggtatgaagggagagaggagaagaggcaaaggtcatgatggtctgtaccaaggtctgtccacagcaacaaaggatacatatgatgctctgcacatgcaggcactcccaccacgg

SEQ ID NO: 8 - DNA, 9679 bp, synthetic construct
  source 1..9679: mol_type = other DNA
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatgctagcggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtagatctagactctagagggtatataatggaagctcgacttccagcttggcattccggtactgttggtaaaaagcttggcaatccggtactgttggtaaaggatccgccaccatgactaacaaatgtctcctccagattgcactgctgctgtgtttctctaccactgctctctccatgagctacaacctgctgggcttcctccaacgctcctccaactttcaatgtcagaaactcctgtggcagctgaatggtcgcctcgaatactgcctcaaggataggatgaacttcgacatccctgaggaaatcaaacagctccagcaattccagaaggaagatgcagctctgaccatctatgagatgctccagaacatcttcgctatctttagacaagactcctccagcacaggatggaatgagacaatcgttgagaacctgctcgctaacgtctaccatcagatcaaccacctgaagaccgtgctggaggagaagctggagaaggaagactttaccagaggcaagctgatgagcagcctgcacctgaagaggtactacggcagaatcctgcactatctgaaggccaaggaatactcccattgcgcatggaccattgtcagagtggagatcctccgcaacttctacttcatcaacaggctgactggctacctgagaaactaagcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactagtgccaccatggactttcaagtgcagatcttcagctttctgctgatctctgctagtgtcatcatgtctgaagtgcaactggtggaatctggaggtggagtggtgcaaccaggtaggtcactcaggctgtcttgctcagcctctggcttcacattctcaggatatggtctgagctgggtcagacaagcaccaggcaagggactggaatgggtcgctatgatcagcagtggaggatcttacacctactatgcagattctgtcaagggtaggtttgcaatctccagagacaatgccaagaacaccctgttcctgcagatggacagcctcagacctgaagatactggagtttacttctgtgctaggcacggagacgatccagcctggtttgcctactggggccaaggaactccagtgacagtctcttctgccagcactaagggtcctagcgtgtttccactggctcccagctccaagtcaacaagtggaggtacagcagctctcggatgtctggtgaaagattacttccctgagcctgtcacagtcagttctggaggtggaggcagtggaggtggtggatctggaggaggagggtctgacatccaactgacacagtcaccatcttcactgtctgccagcgtgggagacagggtcaccattacatgctctgtttcctcttccatatcatcaaacaatctccactggtatcaacagaagcctggtaaggctcccaaaccctggatctatggcacaagcaacctcgcatctggtgtgccatcacgcttctcaggctctgggagtggcacagactacacctttactatctcaagcctgcaacctgaggacattgctacctactattgccagcagtggtcttcatacccttacatgtacacctttggtcagggaactaaggtggaaatcaagagggaaatcgagcagaagctgatttccgaagaggatctcaatggagtgactgtcagcagcgctctgtctaactccatcatgtacttctcacacttcgtgccagtgttcctccctgctaaacccaccacaactccagcacctagacctcccactccagcaccaactattgcatcccagcctctctccctcagaccagaagcatgcagacctgcagcaggtggagctgtgcacacaagaggtctggaccctttctgggtcctcgtggtggtgggtggagtcctggcatgttacagcctcctggtcaccgtggcattcatcatcttctggaagagagggaggaagaagctgctgtacatcttcaaacaaccattcatgaggcctgtgcagacaacccaggaggaagacggatgctcatgcaggttcccagaggaggaggaaggaggttgcgaactcctggagagggtcaagttctcaaggagtgcagatgctccagcctatcaacagggtcagaaccaactgtacaacgagctgaatctcggaagaagagaggagtacgatgtgctggataagagaagaggcagggacccagagatgggtgggaaacccagaagaaagaatcctcaagagggactgtacaatgagctgcagaaggataagatggctgaggcatactcagagatcggtatgaagggagagaggagaagaggcaaaggtcatgatggtctgtaccaaggtctgtccacagcaacaaaggatacatatgatgctctgcacatgcaggcactcccaccacggggttccggagtgaaacagactttgaactttgaccttctcaagttggctggagacgtcgagtccaaccctggtcccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgattaattaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 9 - DNA, 561 bp, synthetic construct
  source 1..561: mol_type = other DNA
atgactaacaaatgtctcctccagattgcactgctgctgtgtttctctaccactgctctctccatgagctacaacctgctgggcttcctccaacgctcctccaactttcaatgtcagaaactcctgtggcagctgaatggtcgcctcgaatactgcctcaaggataggatgaacttcgacatccctgaggaaatcaaacagctccagcaattccagaaggaagatgcagctctgaccatctatgagatgctccagaacatcttcgctatctttagacaagactcctccagcacaggatggaatgagacaatcgttgagaacctgctcgctaacgtctaccatcagatcaaccacctgaagaccgtgctggaggagaagctggagaaggaagactttaccagaggcaagctgatgagcagcctgcacctgaagaggtactacggcagaatcctgcactatctgaaggccaaggaatactcccattgcgcatggaccattgtcagagtggagatcctccgcaacttctacttcatcaacaggctgactggctacctgagaaac

SEQ ID NO: 10 - DNA, 9864 bp, synthetic construct
  source 1..9864: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagccaccatggctcagcggatgacaacacagctgctgctccttctagtgtgggtggctgtagtaggggaggctcagacaaggattgcatgggccaggactgagcttctcaatgtctgcatgaacgccaagcaccacaaggaaaagccaggccccgaggacaagttgcatgagcagtgtcgaccctggaggaagaatgcctgctgttctaccaacaccagccaggaagcccataaggatgtttcctacctatatagattcaactggaaccactgtggagagatggcacctgcctgcaaacggcatttcatccaggacacctgcctctacgagtgctcccccaacttggggccctggatccagcaggtggatcagagctggcgcaaagagcgggtactgaacgtgcccctgtgcaaagaggactgtgagcaatggtgggaagattgtcgcacctcctacacctgcaagagcaactggcacaagggctggaactggacttcagggtttaacaagtgcgcagtgggagctgcctgccaacctttccatttctacttccccacacccactgttctgtgcaatgaaatctggactcactcctacaaggtcagcaactacagccgagggagtggccgctgcatccagatgtggttcgacccagcccagggcaaccccaatgaggaggtggcgaggttctatgctgcagccatgagtggggctgggccctgggcagcctggcctttcctgcttagcctggccctaatgctgctgtggctgctcagcgctagcgaacagaaactcatctccgaagaagacctcaacgcagtgggtcaagacacacaagaagtcatcgtcgtgccacattctctgcccttcaaggtggtggtgatctctgctattctcgcactcgtggtgctcaccatcatctccctgatcattctgatcatgctgtggcagaagaagcctcgcggttctggtgtgaaacagactttgaattttgaccttctcaagttggcgggagacgtcgagtccaaccctgggcccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaatgataacgcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatggatagcactgagaacgtcatcaagcccttcatgcgcttcaaggtgcacatggagggctccgtgaacggccacgagttcgagatcgagggcgtgggcgagggcaagccctacgagggcacccagaccgccaagctgcaagtgaccaagggcggccccctgcccttcgcctgggacatcctgtccccccagttcttctacggctccaaggcgtacatcaagcaccccgccgacatccccgactacctcaagcagtccttccccgagggcttcaagtgggagcgcgtgatgaacttcgaggacggcggcgtggtgaccgtgacccaggactcctccctgcaggacggcaccctcatctaccacgtgaagttcatcggcgtgaacttcccctccgacggccccgtaatgcagaagaagactctgggctgggagccctccactgagcgcaactacccccgcgacggcgtgctgaagggcgagaaccacatggcgctgaagctgaagggcggcggccactacctgtgtgagttcaagtccatctacatggccaagaagcccgtgaagctgcccggctaccactacgtggactacaagctcgacatcacctcccacaacgaggactacaccgtggtggagcagtacgagcgcgccgaggcccgccaccacctgttccagggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 11 - DNA, 8565 bp, synthetic construct
  source 1..8565: mol_type = other DNA
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatgctagcggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtagatctagactctagagggtatataatggaagctcgacttccagcttggcattccggtactgttggtaaaaagcttggcaatccggtactgttggtaaaggatccgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaagcggagctactaacttcagcctgctgaagcaggctggagacgtggaggagaaccctggacctatgcctgccatgaagattgagtgccgcatcacgggaaccctgaacggagtggagtttgagctggtcggaggtggagaagggactcctgagcagggacgtatgaccaacaagatgaagtctaccaagggcgccttgaccttctccccctaccttctctctcatgtcatgggatacgggttctaccactttggtacctatcccagtgggtatgagaatcccttcctgcatgccatcaacaacggggggtacaccaacaccaggattgagaagtatgaggatggaggagttcttcatgttagctttagctacagatatgaagcaggcagggtgattggggatttcaaggttgtcgggacaggattccctgaggacagtgtgatcttcaccgacaagatcatccggtccaatgctaccgtggagcacttgcacccaatgggagacaacgttcttgtgggctccttcgcgagaaccttttccctgagggatggaggctactactcatttgtggttgacagccacatgcacttcaagagtgccatccacccatccatcctccagaacggggggcccatgtttgccttcaggagagttgaggaacttcactccaacactgaacttggcattgtagagtatcaacatgccttcaagactcccatagcatttgcttaagcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 12 - DNA, 8783 bp, synthetic construct
  source 1..8783: mol_type = other DNA
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatgctagcgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactagtgccaccatggactttcaagtgcagatcttcagctttctgctgatctctgctagtgtcatcatgtctgaagtgcaactggtggaatctggaggtggagtggtgcaaccaggtaggtcactcaggctgtcttgctcagcctctggcttcacattctcaggatatggtctgagctgggtcagacaagcaccaggcaagggactggaatgggtcgctatgatcagcagtggaggatcttacacctactatgcagattctgtcaagggtaggtttgcaatctccagagacaatgccaagaacaccctgttcctgcagatggacagcctcagacctgaagatactggagtttacttctgtgctaggcacggagacgatccagcctggtttgcctactggggccaaggaactccagtgacagtctcttctgccagcactaagggtcctagcgtgtttccactggctcccagctccaagtcaacaagtggaggtacagcagctctcggatgtctggtgaaagattacttccctgagcctgtcacagtcagttctggaggtggaggcagtggaggtggtggatctggaggaggagggtctgacatccaactgacacagtcaccatcttcactgtctgccagcgtgggagacagggtcaccattacatgctctgtttcctcttccatatcatcaaacaatctccactggtatcaacagaagcctggtaaggctcccaaaccctggatctatggcacaagcaacctcgcatctggtgtgccatcacgcttctcaggctctgggagtggcacagactacacctttactatctcaagcctgcaacctgaggacattgctacctactattgccagcagtggtcttcatacccttacatgtacacctttggtcagggaactaaggtggaaatcaagagggaaatcgagcagaagctgatttccgaagaggatctcaatggagtgactgtcagcagcgctctgtctaactccatcatgtacttctcacacttcgtgccagtgttcctccctgctaaacccaccacaactccagcacctagacctcccactccagcaccaactattgcatcccagcctctctccctcagaccagaagcatgcagacctgcagcaggtggagctgtgcacacaagaggtctggaccctttctgggtcctcgtggtggtgggtggagtcctggcatgttacagcctcctggtcaccgtggcattcatcatcttctggaagagagggaggaagaagctgctgtacatcttcaaacaaccattcatgaggcctgtgcagacaacccaggaggaagacggatgctcatgcaggttcccagaggaggaggaaggaggttgcgaactcctggagagggtcaagttctcaaggagtgcagatgctccagcctatcaacagggtcagaaccaactgtacaacgagctgaatctcggaagaagagaggagtacgatgtgctggataagagaagaggcagggacccagagatgggtgggaaacccagaagaaagaatcctcaagagggactgtacaatgagctgcagaaggataagatggctgaggcatactcagagatcggtatgaagggagagaggagaagaggcaaaggtcatgatggtctgtaccaaggtctgtccacagcaacaaaggatacatatgatgctctgcacatgcaggcactcccaccacggggttccggagtgaaacagactttgaactttgaccttctcaagttggctggagacgtcgagtccaaccctggtcccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgattaattaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 13 - DNA, 10959 bp, synthetic construct
  source 1..10959: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagccaccatggccttgccaacggctcgacccctgttggggtcctgtgggacccccgccctcggcagcctcctgttcctgctcttcagcctcggatgggtgcagccctcgaggaccctggctggagagacagggcaggaggctgcgcccctggacggagtcctggccaacccacctaacatttccagcctctcccctcgccaactccttggcttcccgtgtgcggaggtgtccggcctgagcacggagcgtgtccgggagctggctgtggccttggcacagaagaatgtcaagctctcaacagagcagctgcgctgtctggctcaccggctctctgagccccccgaggacctggacgccctcccattggacctgctgctattcctcaacccagatgcgttctcggggccccaggcctgcacccgtttcttctcccgcatcacgaaggccaatgtggacctgctcccgaggggggctcccgagcgacagcggctgctgcctgcggctctggcctgctggggtgtgcgggggtctctgctgagcgaggctgatgtgcgggctctgggaggcctggcttgcgacctgcctgggcgctttgtggccgagtcggccgaagtgctgctaccccggctggtgagctgcccgggacccctggaccaggaccagcaggaggcagccagggcggctctgcagggcgggggacccccctacggccccccgtcgacatggtctgtctccacgatggacgctctgcggggcctgctgcccgtgctgggccagcccatcatccgcagcatcccgcagggcatcgtggccgcgtggcggcaacgctcctctcgggacccatcctggcggcagcctgaacggaccatcctccggccgcggttccggcgggaagtggagaagacagcctgtccttcaggcaagaaggcccgcgagatagacgagagcctcatcttctacaagaagtgggagctggaagcctgcgtggatgcggccctgctggccacccagatggaccgcgtgaacgccatccccttcacctacgagcagctggacgtcctaaagcataaactggatgagctctacccacaaggttaccccgagtctgtgatccagcacctgggctacctcttcctcaagatgagccctgaggacattcgcaagtggaatgtgacgtccctggagaccctgaaggctttgcttgaagtcaacaaagggcacgaaatgagtcctcaggtggccaccctgatcgaccgctttgtgaagggaaggggccagctagacaaagacaccctagacaccctgaccgccttctaccctgggtacctgtgctccctcagccccgaggagctgagctccgtgccccccagcagcatctgggcggtcaggccccaggacctggacacgtgtgacccaaggcagctggacgtcctctatcccaaggcccgccttgctttccagaacatgaacgggtccgaatacttcgtgaagatccagtccttcctgggtggggcccccacggaggatttgaaggcgctcagtcagcagaatgtgagcatggacttggccacgttcatgaagctgcggacggatgcggtgctgccgttgactgtggctgaggtgcagaaacttctgggaccccacgtggagggcctgaaggcggaggagcggcaccgcccggtgcgggactggatcctacggcagcggcaggacgacctggacacgctggggctggggctacagggcggcatccccaacggctacctggtcctagacctcagcatgcaagaggccctctcggggacgccctgcctcctaggacctggacctgttctcaccgtcctggcactgctcctagcctccaccctggccgctagcgaacagaaactcatctccgaagaagacctcaacgcagtgggtcaagacacacaagaagtcatcgtcgtgccacattctctgcccttcaaggtggtggtgatctctgctattctcgcactcgtggtgctcaccatcatctccctgatcattctgatcatgctgtggcagaagaagcctcgcggttctggtgtgaaacagactttgaattttgaccttctcaagttggcgggagacgtcgagtccaaccctgggcccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaatgataacgcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatggatagcactgagaacgtcatcaagcccttcatgcgcttcaaggtgcacatggagggctccgtgaacggccacgagttcgagatcgagggcgtgggcgagggcaagccctacgagggcacccagaccgccaagctgcaagtgaccaagggcggccccctgcccttcgcctgggacatcctgtccccccagttcttctacggctccaaggcgtacatcaagcaccccgccgacatccccgactacctcaagcagtccttccccgagggcttcaagtgggagcgcgtgatgaacttcgaggacggcggcgtggtgaccgtgacccaggactcctccctgcaggacggcaccctcatctaccacgtgaagttcatcggcgtgaacttcccctccgacggccccgtaatgcagaagaagactctgggctgggagccctccactgagcgcaactacccccgcgacggcgtgctgaagggcgagaaccacatggcgctgaagctgaagggcggcggccactacctgtgtgagttcaagtccatctacatggccaagaagcccgtgaagctgcccggctaccactacgtggactacaagctcgacatcacctcccacaacgaggactacaccgtggtggagcagtacgagcgcgccgaggcccgccaccacctgttccagggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 14 - DNA, 9806 bp, synthetic construct
  source 1..9806: mol_type = other DNA
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagctagcgaattcgaatttaaatcggatccgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgggaagcggagctactaacttcagcctgctgaagcaggctggagacgtggaggagaaccctggacctatggatagcactgagaacgtcatcaagcccttcatgcgcttcaaggtgcacatggagggctccgtgaacggccacgagttcgagatcgagggcgtgggcgagggcaagccctacgagggcacccagaccgccaagctgcaagtgaccaagggcggccccctgcccttcgcctgggacatcctgtccccccagttcttctacggctccaaggcgtacatcaagcaccccgccgacatccccgactacctcaagcagtccttccccgagggcttcaagtgggagcgcgtgatgaacttcgaggacggcggcgtggtgaccgtgacccaggactcctccctgcaggacggcaccctcatctaccacgtgaagttcatcggcgtgaacttcccctccgacggccccgtaatgcagaagaagactctgggctgggagccctccactgagcgcaactacccccgcgacggcgtgctgaagggcgagaaccacatggcgctgaagctgaagggcggcggccactacctgtgtgagttcaagtccatctacatggccaagaagcccgtgaagctgcccggctaccactacgtggactacaagctcgacatcacctcccacaacgaggactacaccgtggtggagcagtacgagcgcgccgaggcccgccaccacctgttccagtaagcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 15 - DNA, 6309 bp, synthetic construct
  source 1..6309: mol_type = other DNA
ggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattctatgaaatcctttatgggggacccccccctttgtcaaccttgctcaattccttctccccctccgatcctaagactgatttacaagcccgactaaaagggctgcaagcggtgcaggcccaaatctggacacccctggccgaattgtaccggccaggacatccacaaactagccacccatttcaggtgggagactccgtgtacgtccggcggcaccgctctcaaggattggagcctcgttggaagggaccttacatcgtcctgctgaccacgcccaccgccataaaggttgacgggatcgccgcctggattcacgcatcgcacgccaaggcagccccaaaaacccctggaccagaaactcccaaaacctggaagctccgccgttcggagaaccctcttaagataagactctcccgtgtctgactgctaatccaccttgtccctgtactaacccaaaatgaaactcccaacaggaatggtcattttatgtagcctaataatagttcgggcagggtttgacgacccccgcaaggctatcgcattagtacaaaaacaacatggtaaaccatgcgaatgcagcggagggcaggtatccgaggccccaccgaactccatccaacaggtaacttgcccaggcaagacggcctacttaatgaccaaccaaaaatggaaatgcagagtcactccaaaaaatctcacccctagcgggggagaactccagaactgcccctgtaacactttccaggactcgatgcacagttcttgttatactgaataccggcaatgcagggcgaataataagacatactacacggccaccttgcttaaaatacggtctgggagcctcaacgaggtacagatattacaaaaccccaatcagctcctacagtccccttgtaggggctctataaatcagcccgtttgctggagtgccacagcccccatccatatctccgatggtggaggacccctcgatactaagagagtgtggacagtccaaaaaaggctagaacaaattcataaggctatgcatcctgaacttcaataccaccccttagccctgcccaaagtcagagatgaccttagccttgatgcacggacttttgatatcctgaataccacttttaggttactccagatgtccaattttagccttgcccaagattgttggctctgtttaaaactaggtacccctacccctcttgcgatacccactccctctttaacctactccctagcagactccctagcgaatgcctcctgtcagattatacctcccctcttggttcaaccgatgcagttctccaactcgtcctgtttatcttcccctttcattaacgatacggaacaaatagacttaggtgcagtcacctttactaactgcacctctgtagccaatgtcagtagtcctttatgtgccctaaacgggtcagtcttcctctgtggaaataacatggcatacacctatttaccccaaaactggacaggactttgcgtccaagcctccctcctccccgacattgacatcatcccgggggatgagccagtccccattcctgccattgatcattatatacatagacctaaacgagctgtacagttcatccctttactagctggactgggaatcaccgcagcattcaccaccggagctacaggcctaggtgtctccgtcacccagtatacaaaattatcccatcagttaatatctgatgtccaagtcttatccggtaccatacaagatttacaagaccaggtagactcgttagctgaagtagttctccaaaataggaggggactggacctactaacggcagaacaaggaggaatttgtttagccttacaagaaaaatgctgtttttatgctaacaagtcaggaattgtgagaaacaaaataagaaccctacaagaagaattacaaaaacgcagggaaagcctggcatccaaccctctctggaccgggctgcagggctttcttccgtacctcctacctctcctgggacccctactcaccctcctactcatactaaccattgggccatgcgttttcaatcgattggtccaatttgttaaagacaggatctcagtggtccaggctctggttttgactcagcaatatcaccagctaaaacccatagagtacgagccatgagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgt

SEQ ID NO: 16 - DNA, 10816 bp, synthetic construct
  source 1..10816: mol_type = other DNA
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagccaccatggctcagcggatgacaacacagctgctgctccttctagtgtgggtggctgtagtaggggaggctcagacaaggattgcatgggccaggactgagcttctcaatgtctgcatgaacgccaagcaccacaaggaaaagccaggccccgaggacaagttgcatgagcagtgtcgaccctggaggaagaatgcctgctgttctaccaacaccagccaggaagcccataaggatgtttcctacctatatagattcaactggaaccactgtggagagatggcacctgcctgcaaacggcatttcatccaggacacctgcctctacgagtgctcccccaacttggggccctggatccagcaggtggatcagagctggcgcaaagagcgggtactgaacgtgcccctgtgcaaagaggactgtgagcaatggtgggaagattgtcgcacctcctacacctgcaagagcaactggcacaagggctggaactggacttcagggtttaacaagtgcgcagtgggagctgcctgccaacctttccatttctacttccccacacccactgttctgtgcaatgaaatctggactcactcctacaaggtcagcaactacagccgagggagtggccgctgcatccagatgtggttcgacccagcccagggcaaccccaatgaggaggtggcgaggttctatgctgcagccatgagtggggctgggccctgggcagcctggcctttcctgcttagcctggccctaatgctgctgtggctgctcagcgctagcgaacagaaactcatctccgaagaagacctcaacgcagtgggtcaagacacacaagaagtcatcgtcgtgccacattctctgcccttcaaggtggtggtgatctctgctattctcgcactcgtggtgctcaccatcatctccctgatcattctgatcatgctgtggcagaagaagcctcgcggttctggtgtgaaacagactttgaattttgaccttctcaagttggcgggagacgtcgagtccaaccctgggcccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaatgataacgcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatggatagcactgagaacgtcatcaagcccttcatgcgcttcaaggtgcacatggagggctccgtgaacggccacgagttcgagatcgagggcgtgggcgagggcaagccctacgagggcacccagaccgccaagctgcaagtgaccaagggcggccccctgcccttcgcctgggacatcctgtccccccagttcttctacggctccaaggcgtacatcaagcaccccgccgacatccccgactacctcaagcagtccttccccgagggcttcaagtgggagcgcgtgatgaacttcgaggacggcggcgtggtgaccgtgacccaggactcctccctgcaggacggcaccctcatctaccacgtgaagttcatcggcgtgaacttcccctccgacggccccgtaatgcagaagaagactctgggctgggagccctccactgagcgcaactacccccgcgacggcgtgctgaagggcgagaaccacatggcgctgaagctgaagggcggcggccactacctgtgtgagttcaagtccatctacatggccaagaagcccgtgaagctgcccggctaccactacgtggactacaagctcgacatcacctcccacaacgaggactacaccgtggtggagcagtacgagcgcgccgaggcccgccaccacctgttccagggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 17 - DNA, 10108 bp, synthetic construct
  source 1..10108: mol_type = other DNA
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagccaccatggaaactgatacactcctgctctgggttctgctgctgtgggttcctggctctactggtgatgctagcgaacagaaactcatctccgaagaagacctcaacgcagtgggtcaagacacacaagaagtcatcgtcgtgccacattctctgcccttcaaggtggtggtgatctctgctattctcgcactcgtggtgctcaccatcatctccctgatcattctgatcatgctgtggcagaagaagcctcgcggttctggtgtgaaacagactttgaattttgaccttctcaagttggcgggagacgtcgagtccaaccctgggcccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaatgataacgcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatggatagcactgagaacgtcatcaagcccttcatgcgcttcaaggtgcacatggagggctccgtgaacggccacgagttcgagatcgagggcgtgggcgagggcaagccctacgagggcacccagaccgccaagctgcaagtgaccaagggcggccccctgcccttcgcctgggacatcctgtccccccagttcttctacggctccaaggcgtacatcaagcaccccgccgacatccccgactacctcaagcagtccttccccgagggcttcaagtgggagcgcgtgatgaacttcgaggacggcggcgtggtgaccgtgacccaggactcctccctgcaggacggcaccctcatctaccacgtgaagttcatcggcgtgaacttcccctccgacggccccgtaatgcagaagaagactctgggctgggagccctccactgagcgcaactacccccgcgacggcgtgctgaagggcgagaaccacatggcgctgaagctgaagggcggcggccactacctgtgtgagttcaagtccatctacatggccaagaagcccgtgaagctgcccggctaccactacgtggactacaagctcgacatcacctcccacaacgaggactacaccgtggtggagcagtacgagcgcgccgaggcccgccaccacctgttccagggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 18 - DNA, 9156 bp, synthetic construct
  source 1..9156: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagccaccatggaaactgatacactcctgctctgggttctgctgctgtgggttcctggctctactggtgatgctagcgaacagaaactcatctccgaagaagacctcaacgcagtgggtcaagacacacaagaagtcatcgtcgtgccacattctctgcccttcaaggtggtggtgatctctgctattctcgcactcgtggtgctcaccatcatctccctgatcattctgatcatgctgtggcagaagaagcctcgcggttctggtgtgaaacagactttgaattttgaccttctcaagttggcgggagacgtcgagtccaaccctgggcccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaatgataacgcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatggatagcactgagaacgtcatcaagcccttcatgcgcttcaaggtgcacatggagggctccgtgaacggccacgagttcgagatcgagggcgtgggcgagggcaagccctacgagggcacccagaccgccaagctgcaagtgaccaagggcggccccctgcccttcgcctgggacatcctgtccccccagttcttctacggctccaaggcgtacatcaagcaccccgccgacatccccgactacctcaagcagtccttccccgagggcttcaagtgggagcgcgtgatgaacttcgaggacggcggcgtggtgaccgtgacccaggactcctccctgcaggacggcaccctcatctaccacgtgaagttcatcggcgtgaacttcccctccgacggccccgtaatgcagaagaagactctgggctgggagccctccactgagcgcaactacccccgcgacggcgtgctgaagggcgagaaccacatggcgctgaagctgaagggcggcggccactacctgtgtgagttcaagtccatctacatggccaagaagcccgtgaagctgcccggctaccactacgtggactacaagctcgacatcacctcccacaacgaggactacaccgtggtggagcagtacgagcgcgccgaggcccgccaccacctgttccagggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 19 - DNA, 1248 bp, synthetic construct
  source 1..1248: mol_type = other DNA
atgcctgccatgaagattgagtgccgcatcacgggaaccctgaacggagtggagtttgagctggtcggaggtggagaagggactcctgagcagggacgtatgaccaacaagatgaagtctaccaagggcgccttgaccttctccccctaccttctctctcatgtcatgggatacgggttctaccactttggtacctatcccagtgggtatgagaatcccttcctgcatgccatcaacaacggggggtacaccaacaccaggattgagaagtatgaggatggaggagttcttcatgttagctttagctacagatatgaagcaggcagggtgattggggatttcaaggttgtcgggacaggattccctgaggacagtgtgatcttcaccgacaagatcatccggtccaatgctaccgtggagcacttgcacccaatgggagacaacgttcttgtgggctccttcgcgagaaccttttccctgagggatggaggctactactcatttgtggttgacagccacatgcacttcaagagtgccatccacccatccatcctccagaacggggggcccatgtttgccttcaggagagttgaggaacttcactccaacactgaacttggcattgtagagtatcaacatgccttcaagactcccatagcatttgctggaagcggagctactaacttcagcctgctgaagcaggctggagacgtggaggagaaccctggacctatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 20 - DNA, 546 bp, synthetic construct
  source 1..546: mol_type = other DNA
ttctgggtcctcgtggtggtgggtggagtcctggcatgttacagcctcctggtcaccgtggcattcatcatcttctggaagagagggaggaagaagctgctgtacatcttcaaacaaccattcatgaggcctgtgcagacaacccaggaggaagacggatgctcatgcaggttcccagaggaggaggaaggaggttgcgaactcctggagagggtcaagttctcaaggagtgcagatgctccagcctatcaacagggtcagaaccaactgtacaacgagctgaatctcggaagaagagaggagtacgatgtgctggataagagaagaggcagggacccagagatgggtgggaaacccagaagaaagaatcctcaagagggactgtacaatgagctgcagaaggataagatggctgaggcatactcagagatcggtatgaagggagagaggagaagaggcaaaggtcatgatggtctgtaccaaggtctgtccacagcaacaaaggatacatatgatgctctgcacatgcaggcactcccaccacgg